(19) United States
(12) Patent Application Publication
(10) Pub. No.: US 2014/0287044 A1
(43) Pub. Date: Sep. 25, 2014

(54) FIBRIN SEALANT (FIBRINGLURAAS) CONSISTING OF A KIT OF LYOPHILIZED OR FROZEN HIGH CONCENTRATE FRIBINOGEN

(71) Applicant: Rare Antibody Antigen Supply, Inc., Agoura Hills, CA (US)

(72) Inventor: Kieu Hoang, Agura Hills, CA (US)

(73) Assignee: Rare Antibody Antigen Supply, Inc., Agoura Hills, CA (US)

(21) Appl. No.: 14/151,147

(22) Filed: Jan. 9, 2014

Related U.S. Application Data
(63) Continuation-in-part of application No. 13/108,970, filed on May 16, 2011, now abandoned.
Application No. 13/108,970 is a continuation-in-part of application No. 13/064,070, filed on Mar. 4, 2011, now abandoned.

(51) Int. Cl. A61K 38/36; A61K 38/48; A61K 31/44

(52) U.S. Cl. CPC A61K 38/363; A61K 31/44; A61K 38/4833
USPC 424/489; 514/13.6; 424/94.64; 424/649

(57) ABSTRACT

The application is directed to a fibrin sealant (FIBRINGLURAAS®) consisting of a kit of lyophilized or frozen high concentrate fribinogen in which 5% a1at will be added into the final bulk and or 5% a1at as a diluent for high concentrate fibrinogen and new found proteins kh30, kh31, kh32, kh44, kh46, kh47, and kh52 in which the kh good healthy cells are present, either non-heated or heating to at least 1° C. and above, preferably at least 101° C., and lyophilized or frozen thrombin used to compound glue membrane, the diameter of which is less than 10 micrometers the actual size of the glue membrane of the fibrin sealant (FIBRINGLURAAS®) is from 0.6 μm, to 101° C. heating 0.005 micrometers and its topical applications for all solid tumor cancer

### CROSS REFERENCE TO RELATED APPLICATIONS

This application is a continuation-in part of U.S. patent application Ser. No. 13/108,970, filed May 6, 2011, entitled “Fibrin Sealant Consisting of A kit of Lyophilized High Concentrate . . . ”, which is continuation in part of U.S. patent application Ser. No. 13/064,070, filed Mar. 4, 2011, entitled “Fibrin Sealant Consisting of a Kit of Lyophilized . . . ”, each of which applications are incorporated herein by reference.

### FIELD OF INVENTION

The present application is to a Fibrin Sealant consisting of a kit of lyophilized or frozen high concentrate Fibrinogen in which 5% A1At will be added into the final bulk and or 5% A1At as a diluent for high concentrate Fibrinogen and new found proteins KH30, KH31, KH32, KH44, KH46, KH47, and KH52 in which the KH good healthy cells are present, either non-heated or heating to at least 1° C. and above, preferably at least 101° C., and lyophilized or frozen Thrombin used to compound glue membrane, the diameter of which is less than 10 micrometers the actual size of the glue membrane of the Fibrin Sealant is from 0.6 μm, to 101° C. heating 0.005 micrometers and its topical applications for all solid tumor cancer.

The present application relates to alpha 1 antitrypsin, thrombin and high concentrate fibrinogen. The use of 5% alpha 1 antitrypsin is as a diluent or the use of 5% alpha 1 antitrypsin final bulk to add into high concentrate fibrinogen, in order to stabilize, to enhance in to increase the density of the membrane. It also prolongs the life of the membrane up to 59 days in the body of the mice and even at 59 days when the surgical operation to observe the membrane, the membrane still exists in the study of the 18 mice for breast cancer.

Fibrin Sealant consisting of a kit of lyophilized or frozen high concentrate fibrinogen in which 5% A1AT will be added into the final bulk and or 5% A1AT as a diluent for lyophilized or frozen high concentrate fibrinogen, either non-heated or heating to at least 1° C. and above, preferably at least 101° C., and lyophilized or frozen thrombin used to compound glue membrane, the diameter of which is less than 10 micrometers the actual size of the glue membrane of the fibrin sealant is from 0.6 μm, to 101° C. heating 0.005 micrometers.
Thrombin, a protein, contains good healthy cells.
High concentrate fibrinogen, another protein, contains good healthy cells.
AFOD (HDL ApoAl), another protein, contains good healthy cells and its topical applications for all solid tumor cancer.

### DESCRIPTION OF INVENTION AND ITS PURPOSES

1. A kit of lyophilized or frozen high concentrated fibrinogen (hcfng) in which 5% A1AT will be added into the final bulk and or 5% A1AT as a diluent for lyophilized or frozen high concentrate fibrinogen and either not heated or dry, wet or vapor heated up to at least 1° C., preferably at least 101° C., during the purification process of the high concentrated fibrinogen (HcFNG) which is used in the kit of Fibrin Sealant is much different than a regular fibrinogen under the trade name FibroRAAS.

High concentrate Fibrinogen contains Factor XIII, clottable fibrinogen purity should be equal to or higher than 80%. Clotting activity should be less or equal to 60 seconds.

And concentration range from 5% to 9% (five to nine percent) and should be Topical use whereas regular Fibrinogen can be injected and need an osmotic pressure equal or greater than 240 m0smol/kg and purity is only equal or less than 70% and Concentration is ONLY 2% (two percent).
Fibrin Sealant has been used as topical hemostasis drug in the treatment of the surface of burns, abdominal incisions of general surgery, oozing of blood in liver operations, and blood vessel surgery to stop bleeding.
Fibrinogen can be injected and use in the treatment of:
1. Congenital hypofibrinogenaemia or fibrinogenaemia.
2. Acquired hypofibrinogenaemia; severe liver damage, cirrhosis, disseminated intravascular coagulation, disorder of blood coagulation due to the lacking fibrinogen caused by obstetric hemorrhage, big surgical, trauma or internal hemorrhage.
   A kit of lyophilized or frozen High concentrate Fibrinogen or frozen described above and lyophilized or frozen thrombin is used to compound glue membrane, the kit and the glue membrane, the diameter of the glue membrane mesh is smaller than human cancer cells, which are of the size of 10-100 micrometer. Preferably, the glue membrane mesh is 0.005 micrometer or less in its biggest dimension, and far smaller than human tumor cells of 10-100 micrometers. The glue membrane can prevent cancer cells from becoming detached and spreading into the abdominal cavity during the surgical operations of gastrointestinal cancer in mice. Such a glue membrane has clinical applications other than gastric and gastrointestinal cancer in humans, such as colon and breast cancer, and all kinds of solid tumors that have not yet spread to other parts of body, such as AIDS related cancers, osteosarcoma, and cancers of the anus, appendix, bile duct, bladder, brain, breast, cervix, colon, esophagus, eye, gall bladder, head, neck, heart, liver, kidney, larynx, lip, oral cavity, lung, mouth, paranasal sinus and nasal cavity, ovaries, pancreas, parathyroid, penis, prostate, rectum, salivary glands, skin, spleen, throat, testicles, urethra, and vagina, as well as renal cell carcinoma.

2. The compound for the glue membrane according to the present invention should be applied topically and should not be injected and should not apply for leukemia's, including acute myeloid leukemia (MO-M7), lymphoma, marrow malignancy, acute lymphoid leukemia (small, middle, large), myeloid dysfunction syndrome (MDS), anemia, lupus and sclerosis in the brain.

3. If used alone, the fibrin sealant membrane can trap the cancer cells. This means that the fibrin sealant membrane separates tumor cells from healthy tissue, and that blood vessels cannot reach cancer cells, so that theoretically the fibrin sealant membrane can inhibit cancer cells from releasing cytokines, including TNF (Tumor Necrosis Factor), and activation of histones to healthy tissues. In other words, the fibrin sealant membrane, if used alone, can inhibit the toxicity produced by only one time radio-chemotherapy because the fibrin sealant membrane can trap cancer cells and hold them back, so that it prevents the invasion of tumor cells into healthy tissue. The fibrin sealant membrane can be used as an adjuvant instrument to prevent cancer cells from becoming detached and spreading into the abdominal cavity during surgical operations of gastrointestinal cancer.

4. The high concentrated fibrinogen, can be combined with agent such as fluorouracil (C4H3FN202), available under the name RAAS 1 to 45 FU, which can inhibit the tumor cells. With this combination, the overall effect is that the GROWTH of TUMORS will be INHIBITED. Fluorouracil is a pyrimidine analog which is used in the treatment of cancer. It works through noncompetitive inhibition of thymidylate synthase.

It was designed, synthesized and patented by Charles Heidelberger in 1957. When flourouracil (C4H3FN202) is applied together with the fibrin sealant membrane in vivo, the molecules of flourouracil are also trapped in the fibrin sealant membrane, which keeps the drug molecules stored in the membrane so that they can be released slowly. This effect also makes the drug much more concentrated locally and less toxic, when compared with injecting the drug intravenously. Fluorouracil is just one agent that can inhibit the tumor cells and perform other tasks as described above. Other agents that can inhibit the tumor cells can be used instead of, or in addition to, fluorouracil. The agents that can inhibit the tumors that are contemplated for use with the present invention include all presently known such agents, as well as all such agents that will become available in the future. In order to prevent toxicity produced by only a one time chemo-radio therapy immediately follow by a surgical operations to remove cancer tumors, then Fibrinogen can be applied topically alone or in combination with fluorouracil (C4H3FN202), which can completely kill Cancer Tumor cells after the surgical operations.

5. If Fibrinogen is used alone; throat cancer patients will not loose their taste, because no chemo-radio therapy is used.

6. Cancer cells may secrete TNF, various other cytokines, special proteins, even proteinase or some special hormones, depending on the type of cancer. For example, some liver cancer cells secrete alpha fetal protein. Some breast cancer, lung cancer and prostate cancer cells can release cytokines like interleukins. As the cancer cells are trapped in the fibrin sealant membrane and later killed by chemotherapy reagents combined with the fibrin sealant, secreted TNF, various other cytokines, special proteins, even proteinase or some special hormones can also be trapped in the fibrin sealant membrane. MAT in the fibrin sealant inhibits the activity of some proteinase secreted by cancer cells which are nearby healthy cells.

### BACKGROUND OF THE INVENTION

A. Field of invention

The present invention relates to a kit of lyophilized or frozen thrombin and lyophilized or frozen high concentrated fibrinogen and its usage to prevent tumor cell pervasion caused by incision and trauma in tumor operations. The present invention addresses a number of problems. For example, in tumor operations, incision and trauma usually cause tumor cell pervasion that can increase the risk of tumor palindromia and metabasis after tumor operations and shorten the life span of the patients.

A glue membrane made from a compound of the high concentrated fibrinogen and thrombin, according to the present invention, is applied to prevent pervasion of tumor cells caused by incision and trauma in a tumor surgical operation. The glue membrane can reduce the risk of palindromia and metabasis of the tumor after the operation and increase the life span of the patient. It is possible that, due to the glue membrane to stop the dissemination of tumor cells, cancers will not reoccur.

The high concentrated fibrinogen, can be combined with an agent such as fluorouracil (C4H3FN202), as an adjuvant instrument to prevent cancer cells from becoming detached and spreading into the abdominal cavity during surgical operation of gastrointestinal cancer, and the fluorouracil in the resultant glue membrane will kill the tumor cancer cells.

There is a tendency for lumps to develop where the mamma or a lump has been removed, after radical mamma and lump exsection. Daubing the high concentrated fibrinogen and thrombin that form the glue membrane according to the present invention in the places of removal of the mamma or a lump reduces the number and/or size of lumps that tend to form there.

A compound of lyophilized or frozen thrombin and high concentrated fibrinogen has also been used as a topical hemostasis drug in the treatment of the surface of burns, abdominal incisions of general surgery, oozing of blood in liver operations, and blood vessel surgery.

### SUMMARY OF THE INVENTION

In accordance with the present invention, a compound of lyophilized or frozen thrombin and high concentrated fibrinogen in which 5% A1AT will be added into the final bulk and or 5% A1AT as a diluent for lyophilized or frozen high concentrate fibrinogen and either not heated or dry, wet or vapor heated up to at least 1° C., preferably at least 101° C., during the purification process of the high concentrated fibrinogen is used to compound a glue membrane that is used to prevent dissociative tumor cell pervasion. The A1AT will be processed separately and concentrated up to 5% into the non-sterile final bulk which will be added into the non-sterile final bulk of the high concentrate fibrinogen to maximize the stability and density of the membrane.

A kit of lyophilized or frozen thrombin and high concentrated fibrinogen in accordance with the present invention produces a solid-meshy glue membrane to prevent dissociative tumor cell pervasion and to prevent cancer cells from becoming detached and spreading into the abdominal cavity during surgical operations of gastrointestinal cancer in mice and other clinical applications, such as colon and breast cancer, and it can be used for all kinds of solid tumors.

Fibrinogen can inhibit the release of cytokines, including TNF (Tumor Necrosis Factor), stop the activation of histones and toxicity produced by radio-chemo therapies to prevent the deaths of cancer patients due to the release of cytokines, TNF and stop the activation of histones and toxicity produced by radio-chemo therapies if used alone.

The high concentrated fibrinogen can be combined with an agent such as fluorouracil (C4H3FN202), as an adjuvant instrument to kill cancer cells, and the fluorouracil in the resultant glue membrane will kill the tumor cancer cells.

The Fibrin Sealant can be combined with as agent such as Sorafenib as an adjuvant instrument to kill the breast cancer cells.

Fibrinogen with Thrombin and high concentrated Fibrinogen in which 5% of Alpha 1 is added or 5% of Alpha 1 as a diluent containing the following KH proteins, namely KH30 (SEQ ID NO: 1), KH31 (SEQ ID NO: 2), KH32 (SEQ ID NO: 3), KH44 (SEQ ID NO: 5), KH46 (SEQ ID NO: 7), KH47 (SEQ ID NO: 8), AND KH52 (SEQ ID NO: 4) inhibit the growth of all cancer tumors.

### BRIEF DESCRIPTION OF THE DRAWINGS

Other objects and features of the present invention will become apparent from the following detailed description considered in connection with the accompanying drawings. It should be understood, however that the drawings are designed for the purpose of illustration only and not as a definition of the limits of invention.

FIG. 1 and FIG. 2 are electron micrographs of a glue membrane compounded by daubing a solution of lyophilized or frozen thrombin and a solution of lyophilized or frozen high concentrated fibrinogen one layer after another on a glass slide in connection with an in vitro study.

FIG. 3A1 and FIG. 3A2 are electron micrographs of a control without a glue membrane.

FIGS. 3B1 and 3B2 are electron micrographs of a material surface with a glue membrane according to the present invention in a tumor cell pervasion experiment.

FIG. 3C1 and FIG. 3C2 are electron micrographs of a glue membrane in a tumor cell pervasion experiment involving a glue membrane treatment according to the present invention.

FIG. 3D1 is a photograph of a gel membrane stability test comparing the enhanced stability of FNG with A1AT as opposed to FNG without A1AT over 65 hours.

FIG. 3D2 is a photograph of the results of an eight-hour western blot gel membrane degradation test. It can be seen that the degradation of an FS glue membrane accelerated when A1AT in FNG is neutralized by an A1AT antibody.

FIG. 3D3 shows lung cancer pervasion on a control glue membrane untreated with FS.

FIG. 3D4 shows inhibited lung cancer pervasion involving a glue membrane treated with FS.

FIG. 3D5 is an electron micrograph of lung cancer cells in medium.

FIG. 3D6 and FIG. 3D7 are pictures comparing lung cancer pervasion on FIBIGLURAAS and pig plasma derived FS.

FIG. 4A is an electron micrograph of an FS gel membrane at a magnification of 27,500 with visible pores of the gel membrane less than 0.1 μm in diameter.

FIG. 4B is an electron micrograph of an FS gel membrane at a magnification of 15,000 with visible pores of the gel membrane about 0.1-0.2 μm in diameter.

FIG. 4C is an electron micrograph of an FS gel membrane at a magnification of 3,810 with essentially no visible pores on the gel membrane.

FIG. 4D is an electron micrograph of an FS gel membrane at a magnification of 1,600 with essentially no visible pores on the gel membrane.

FIG. 5A, FIG. 5B, FIG. 5C, FIG. 5D, AND FIG. 5E are typical electron micrographs of FS gel membrane at a magnification of 27,000 treated at different temperatures. In FIG. 5A the gel membrane is treated at 0° C. In FIG. 5B the gel membrane is treated at 30° C. In FIG. 5C the gel membrane is treated at 60° C. In FIG. 5D the gel membrane is treated at 90° C. In FIG. 5E the gel membrane is treated at 101° C.

FIG. 6 is a photograph of a western blot in which A1AT is enriched in high concentration fibrinogen. The A1AT protein was detected using a polyclonal antibody against human A1AT. “TB” and “FNG” stand for the thrombin and high concentrated fibrinogen. TB was used for a negative control. “A1AT standard” stands for a commercially available A1AT as a positive control.

FIG. 7 is a photograph of a western blot in which the degradation of an FS glue membrane accelerated when A1AT in FNG is neutralized by an A1AT antibody. An A1AT polyclonal antibody was added to the high concentrated fibrinogen. “−” represents Fibrinogen+WFI, which means A1AT will keep its activity. “+” represents Fibrinogen+anti-A1AT antibody, which means A1AT is neutralized. The degradation of FS glue membranes is detected using SDS-PAGE (sodium dodecyl sulfate). FIG. 7 illustrates the degradation of FS glue membranes begins as early as 15 minutes after the glue membrane is formed when the A1AT is neutralized by addition of the antibody. The difference is also present at 2 hours, 4 hours, and 8 hours after the glue membrane is formed.

FIG. 8 is a photograph of a western blot demonstrating that the enrichment of A1AT in the purification process of high concentrated fibrinogen makes FS glue membrane much more stable. The degradation of FS glue membrane is compared between fibrinogen (FNG) and A1AT enriched high concentrated fibrinogen (QFNG). FIG. 8 shows the degradation of FS glue membrane with A1AT enriched FNG will not begin within the first 8 hours after the gel is formed, which means it is much more stable.

FIG. 9 is a photograph comparing the condition and stability of FNG and QFNG FS glue membranes 48 hours after they are formed. The glue membrane is still in a stable status when using A1AT-enriched high concentrated fibrinogen. However, the glue membrane of FNG has already degraded.

FIG. 10 is a graph comparing the in vitro inhibitory effect on gastric cancer cells of Thrombin (“TB”), high concentrate fibrinogen, and HDL ApoAl (“AFOD”), versus control.

FIG. 11 is a graph comparing the in vitro inhibitory effect on gastric cancer cells of TB and AFOD, versus control.

FIG. 12A is a photograph of gastric cancer cells treated with 5 U/mL TB.

FIG. 12B is a photograph of gastric cancer cells treated with 25 U/mL TB.

FIG. 12C is a photograph of the untreated control for gastric cancer cells.

FIG. 13 is a graph comparing the in vitro inhibitory effect on cervical cancer cells of TB versus control in relation to protein expression and treatment dose.

FIG. 14A is a photograph of cervical cancer cells treated with 5 U/mL TB.

FIG. 14B is a photograph of cervical cancer cells treated with 25 U/mL TB.

FIG. 14C is a photograph of the untreated control for cervical cancer cells.

FIG. 15 is a graph comparing the in vitro inhibitory effect on breast cancer cells of TB and AFOD, versus control.

FIG. 16A is a photograph of breast cancer cells treated with 5 U/mL TB.

FIG. 16B is a photograph of breast cancer cells treated with 25 U/mL TB.

FIG. 16C is a photograph of the untreated control for breast cancer cells.

FIG. 17 is a graph comparing the in vitro inhibitory effect on ovarian cancer cells of TB and AFOD, versus control.

FIG. 18A is a photograph of ovarian cancer cells treated with 5 U/mL TB.

FIG. 18B is a photograph of ovarian cancer cells treated with 25 U/mL TB.

FIG. 18C is a photograph of the untreated control for ovarian cancer cells.

FIG. 19 is a graph comparing the in vitro inhibitory effect on lung cancer cells of TB, versus control.

FIG. 20A is a photograph of lung cancer cells treated with 5 U/mL TB.

FIG. 20B is a photograph of lung cancer cells treated with 25 U/mL TB.

FIG. 20C is a photograph of the untreated control for lung cancer cells.

FIG. 21 is a graph comparing the in vitro inhibitory effect on esophageal cancer cells of TB, versus control.

FIG. 22A is a photograph of esophageal cancer cells treated with 5 U/mL TB.

FIG. 22B is a photograph of esophageal cancer cells treated with 25 U/mL TB.

FIG. 22C is a photograph of the untreated control for lung cancer cells.

FIG. 23 is a graph comparing the in vitro inhibitory effect on liver cancer cells of TB, versus control.

FIG. 24A is a photograph of liver cancer cells treated with 5 U/mL TB.

FIG. 24B is a photograph of liver cancer cells treated with 25 U/mL TB.

FIG. 24C is a photograph of the untreated control for liver cancer cells.

FIG. 25 is a graph comparing the in vitro inhibitory effect on pancreas cancer cells of TB, versus control.

FIG. 26A is a photograph of pancreas cancer cells treated with 5 U/mL TB.

FIG. 26B is a photograph of pancreas cancer cells treated with 25 U/mL TB.

FIG. 26C is a photograph of the untreated control for pancreas cancer cells.

FIG. 27 is a graph comparing the in vitro inhibitory effect on leukemia cancer cells of TB, versus control.

FIG. 28A is a photograph of leukemia cancer cells treated with 5 U/mL TB.

FIG. 28B is a photograph of leukemia cancer cells treated with 25 U/mL TB.

FIG. 28C is a photograph of the untreated control for leukemia cancer cells.

FIG. 29A is a photograph of the untreated control for gastric cancer cells.

FIG. 29B is a photograph of gastric cancer cells treated with 0.1% AFOD.

FIG. 29C is a photograph of gastric cancer cells treated with 0.5% AFOD.

FIG. 29D is a photograph of gastric cancer cells treated with 2.5% AFOD.

FIG. 30A is a photograph of the untreated control for cervical cancer cells.

FIG. 30B is a photograph of cervical cancer cells treated with 0.1% AFOD.

FIG. 30C is a photograph of cervical cancer cells treated with 0.5% AFOD.

FIG. 30D is a photograph of cervical cancer cells treated with 2.5% AFOD.

FIG. 31A is a photograph of the untreated control for breast cancer cells.

FIG. 31B is a photograph of breast cancer cells treated with 0.1% AFOD.

FIG. 31C is a photograph of breast cancer cells treated with 0.5% AFOD.

FIG. 31D is a photograph of breast cancer cells treated with 2.5% AFOD.

FIG. 32A is a photograph of the untreated control for ovarian cancer cells.

FIG. 32B is a photograph of ovarian cancer cells treated with 0.1% AFOD.

FIG. 32C is a photograph of ovarian cancer cells treated with 0.5% AFOD.

FIG. 32D is a photograph of ovarian cancer cells treated with 2.5% AFOD.

FIG. 33A is a photograph of the untreated control for lung cancer cells.

FIG. 33B is a photograph of lung cancer cells treated with 0.1% AFOD.

FIG. 33C is a photograph of lung cancer cells treated with 0.5% AFOD.

FIG. 33D is a photograph of lung cancer cells treated with 2.5% AFOD.

FIG. 34A is a photograph of the untreated control for esophageal cancer cells.

FIG. 34B is a photograph of esophageal cancer cells treated with 0.1% AFOD.

FIG. 34C is a photograph of esophageal cancer cells treated with 0.5% AFOD.

FIG. 34D is a photograph of esophageal cancer cells treated with 2.5% AFOD.

FIG. 35A is a photograph of the untreated control for liver cancer cells.

FIG. 35B is a photograph of liver cancer cells treated with 0.1% AFOD.

FIG. 35C is a photograph of liver cancer cells treated with 0.5% AFOD.

FIG. 35D is a photograph of liver cancer cells treated with 2.5% AFOD.

FIG. 36A is a photograph of the untreated control for pancreas cancer cells.

FIG. 36B is a photograph of pancreas cancer cells treated with 0.1% AFOD.

FIG. 36C is a photograph of pancreas cancer cells treated with 0.5% AFOD.

FIG. 36D is a photograph of pancreas cancer cells treated with 2.5% AFOD.

FIG. 37A is a photograph of the untreated control for leukemia cancer cells.

FIG. 37B is a photograph of leukemia cancer cells treated with 0.1% AFOD.

FIG. 37C is a photograph of leukemia cancer cells treated with 0.5% AFOD.

FIG. 37D is a photograph of leukemia cancer cells treated with 2.5% AFOD.

FIG. 38 shows the four regions of a mouse peritoneal surface of the abdomen and pelvis involved with establishment of the modified peritoneal cancer index (PCI).

FIG. 39A depicts the dissected peritoneal surface of the abdomen and pelvis of a mouse in the control group.

FIG. 39B depicts the dissected peritoneal surface of the abdomen and pelvis of a mouse in the FS treatment group with small, isolated and well-capsulized tumor nodules.

FIG. 39C depicts the dissected peritoneal surface of the abdomen and pelvis of a mouse in the Sino-Fuan treatment group with small nodules, and some un-degraded agents left.

FIG. 39D depicts the dissected peritoneal surface of the abdomen and pelvis of a mouse in the FS plus Sino-Fuan combination treatment group with only tiny tumor nodules.

FIG. 40 is a graph showing the anti-tumor efficacy of high concentrated fibrinogen enriched at A1AT thrombin and AFOD in PDX colorectal cancer model CO-04-0002.

FIG. 41 is a graph showing the anti-tumor efficacy of high concentrated fibrinogen enriched at A1AT thrombin and AFOD in PDX colorectal cancer model CO-040002 and CO-04-0001.

FIG. 42 is photographs of tumors dissected from the abdominal cavity of mice in colorectal cancer model CO-04-0002 (upper panels) and CO-04-0001 (bottom panel) superimposed on a chart ordered by treatment group.

FIG. 43 is a graph showing relative change of body weight (%) by different treatment groups in colorectal cancer model CO-04-0002. Data are expressed as mean±SEM. Relative change of body weight (RCBW) was calculated based on the following formula: RCBW (%)=(BWi−BW0)/BW0×100%; BWi was the body weight on the day of weighing and BW0 was the body weight before surgery.

FIG. 44 is a graph showing anti-tumor efficacy of high concentrated fibrinogen enriched A1AT thrombin and AFOD in PDX lung cancer model LU-01-0032. Tumor weights from model LU-01-0032 were used. Data are expressed as mean±SEM. *<0.05, **<0.01, ***<0.001 vs vehicle group (one-way ANOVA and Dunnett's test).

FIG. 45 is photographs of tumors dissected from the abdominal cavity of mice in the lung cancer model LU-01-0032 superimposed on a chart organized by treatment group. Tumors from each mouse of model LU-01-0032 were pooled and weighed; the scale bar being 1 cm. The treatment groups on the chart are A: sham-operated, B: vehicle control, C: matrigel, D: test agent high dose, E: test agent moderate dose, and F: test agent low dose.

FIG. 46 is a graph showing ratios of mice with palpable tumors observed from each group in lung cancer model LU-01-0032.

FIG. 47 is a graph showing relative change of body weight (%) by different treatment groups in lung cancer model LU-01-0032. Data are expressed as mean±SEM. Relative change of body weight (RCBW) was calculated based on the following formula: RCBW (%)=(BWi−BW0)/BW0×100%; BWi was the body weight on the day of weighing and BW0 was the body weight before surgery.

FIG. 48 is photographs of tumors dissected from mice in the hepatic cancer model 28 days after implantation.

FIG. 49 is a table describing treatment group, number of mice in each group, tumor weight, and remarks for the hepatic cancer model.

FIG. 50 reflects data and shows tumor pictures from mice in three treatment groups in the hepatic cancer model 54 days after implantation.

FIG. 51 is four pictures of mice 3-2 in group three of the hepatic cancer model 54 days after implantation.

FIG. 52 is four pictures of mice 3-3 in group three of the hepatic cancer model 54 days after implantation.

FIG. 53 is four pictures of mice 5-1 in group five of the hepatic cancer model 54 days after implantation.

FIG. 54 is four pictures of mice 5-2 in group five of the hepatic cancer model 54 days after implantation.

FIG. 55 is four pictures of mice 5-3 in group five of the hepatic cancer model 54 days after implantation.

FIG. 56 is four pictures of mice 6-1 in group six of the hepatic cancer model 54 days after implantation.

FIG. 57 is four pictures of mice 6-3 in group six of the hepatic cancer model 54 days after implantation.

### DETAILED DESCRIPTION OF THE PREFERRED EMBODIMENTS

The effects of fibrinogen and thrombin on the process of thrombosis are well known.

There are many products from fibrinogen and thrombin, but the products are used to stanch after operations in most cases.

For colon cancer, the data show that high concentrated fibrinogen enriched alpha 1 antitrypsin, thrombin and AFOD at all 3 doses exhibits significant inhibitory effects on tumor growth in the PDX colorectal cancer model while no significant toxicity was observed. This indicates that high concentrated fibrinogen enriched with alpha 1 antitrypsin, thrombin and AFOD is a potential anti-tumor agent in colorectal cancer, warranting further development of the agent for clinical applications.

For lung cancer, the data show that concentrated fibrinogen enriched alpha 1 antitrypsin, thrombin and AFOD at all 3 doses exhibits significant inhibitory effects on tumor growth in the lung cancer model, while no significant toxicity was observed. This indicates that high concentrated fibrinogen enriched with alpha 1 antitrypsin, thrombin and AFOD was a potential anti-tumor agent in lung cancer, warranting further development of high concentrated fibrinogen enriched, alpha one antitrypsin, thrombin and AFOD for clinical applications.

For breast cancer, a BEL-7404 peritoneal implantation hepatic cancer model was used to evaluate the anti-cancer efficacy of fibrin sealant at 2 ml/mouse. On day 21, after implantation, all mice in vehicle group and positive groups exhibited palpable tumors, while no mice in the FS group exhibited any palpable tumors. On day 28 after implantation, the tumors in the vehicle group reached 0.88 g, while the fibrin sealant group was only 0.06. On day 52, after implantation, mice treated with FS did not exhibit any palpable tumors. The results showed that FS had inhibition on tumor growth. No body weight loss or toxicity were found in the FS treated groups, which showed that FS had no side effects.

With the above referenced three studies, the efficacy of Fibrinogen in this application has been proven efficacious.

We know for certain that the growth of cancer cells, but it has turned into a good KH healthy cell that synthesizes a good KH protein against the cancer.
A good healthy KH protein like thrombin, alpha 1 antitrypsin and HCFNG with KH protein characteristics with their sequence synthesized by a good KH healthy cell can do the following:
   send signals to damaged, sick, and bad cells that triggers the synthesis of good proteins that transforms these cells to become good healthy cells; send signals to other currently undamaged cells to synthesize good proteins to protect them from being damaged, infected and prone to DNA and other cellular alterations; and send signals to the body to produce new cells that are healthy and prevent them from being affected by intra-cellular and extracellular damaging signals. The mechanisms that govern these processes are the KH good healthy cell processes that make good proteins to boost immune system in order to cure, to protect and to prevent diseases, viral infections, bacterial infections, autoimmune diseases, neurological disorders, all types of solid and blood cancers, coagulation, diabetic, inhibitor, immune deficiency, muscle and nerve repair and restoration from human, animal or other substances by the method of fractionation, purification, recombinant DNA, monoclonal antibody, transgenic and expressions of cells from the culture of good healthy cells.

New found proteins KH21, KH22, KH23, KH24, KH25, KH26, KH27, KH30, KH31, KH32, KH44, KH45, KH46, KH47, KH48, KH49, KH50, and KH52 are present in good healthy KH cells in different existing and newly developed RAAS plasma derived medicine products, recombinant DNA, engineered DNA, eDNA, monoclonal and natural products, or synthesized products and their application.

Protein KH21 has amino acid sequence SEQ ID NO: 9.

Protein KH22 has amino acid sequence SEQ ID NO:10.

Protein KH 23 has amino acid sequence SEQ ID NO: 11.

Protein KH24 has amino acid sequence SEQ ID NO: 12.

Protein KH25 has amino acid sequence SEQ ID NO: 13.

Protein KH26 has amino acid sequence SEQ ID NO: 14.

Protein KH27 has amino acid sequence SEQ ID NO: 15.

Protein KH30 has amino acid sequence SEQ ID NO: 1.

Protein KH31 has amino acid sequence SEQ ID NO: 2.

Protein KH32 has amino acid sequence SEQ ID NO: 3.

Protein KH44 has amino acid sequence SEQ ID NO: 5.

Protein KH45 has amino acid sequence SEQ ID NO: 6.

Protein KH46 has amino acid sequence SEQ ID NO: 7.

Protein KH47 has amino acid sequence SEQ ID NO: 8.

Protein KH48 has amino acid sequence SEQ ID NO: 16.

Protein KH49 has amino acid sequence SEQ ID NO: 17.

Protein KH50 has amino acid sequence SEQ ID NO: 18.

Protein KH52 has amino acid sequence SEQ ID NO: 4.

According to the present invention, a solid-meshy membrane compounded by lyophilized or frozen thrombin and high concentrated fibrinogen that is intentionally enriched and preserved with the fibrinolysis inhibitor alpha 1-antitrypsin (A1AT) during the purification of the high concentrated fibrinogen and either not heated or dry, wet or vapor heated up to at least 1° C., preferably at least 101° C., to intensify the stability and durability of the high concentrated fibrinogen has a mesh that is smaller than human cancer cells, which are of the size of 10-100 μm. Preferably, the glue membrane mesh is 0.6 μm in its biggest dimension, and far smaller than human tumor cells of 10-100 μm.

The high concentrated fibrinogen and thrombin are sourced from human plasma tested by nucleic acid testing (NAT) to be negative for HIV 1&2, hepatitis B (HBV) and hepatitis C (HCV). Both enveloped viruses and non-enveloped viruses are inactivated by a solvent detergent step (S/D) using tri(n-butyl)phosphate (TNBP) and Tween 80; nanofiltration is used to remove both enveloped viruses and non-enveloped viruses for the thrombin; and either not heating or dry, wet or vapor heating up to at least 1° C., preferably at least 101° C. for 30 minutes is done to inactivate the non-enveloped viruses for the high concentrated fibrinogen. All viruses, both enveloped and non-enveloped, are inactivated by these steps. Both components, high concentrated fibrinogen and thrombin, are lyophilized or frozen and put into separate solutions.

### In Vitro Study:

In experiments using the present invention, a thin and smooth layer of high concentrated fibrinogen solution is daubed on the surface of a glass slide, or on the bottom surfaces of cell culture inserts in an assay kit, to form a coating. After about 5 seconds, a thin and smooth layer of thrombin solution is daubed sequentially on the high concentrated fibrinogen coating. The daubing of the high concentrated fibrinogen solution and then the thrombin solution is repeated about 3-5 times. A solid-meshy glue membrane forms quickly. The diameter of the glue membrane mesh is smaller than human cancer cells, which are of the size of 10-100 μm. Preferably, the glue membrane mesh is 0.6 μm or less in its biggest dimension, as little as 0.0 μm, and far smaller than human tumor cells of 10-100 μm. The glue membrane can hold back the human tumor cells and prevent pervasion.

Thus, the high concentrated fibrinogen and thrombin solutions are daubed alternately with one another on the local surface of tumor tissue, one layer at a time. The solid-meshy glue membrane that forms on the local tissue surface prevents dissociative tumor cell pervasion in operations, reduces the risk of palindromia and metabasis of tumors after treatment, and improves the life span of patients, and possibly cancer will not reoccur. The glue membrane has a good biological compatibility and convenient usage.

### Example 1

### Generation of Glue Membrane

(1) The high concentrated fibrinogen solution and the thrombin solution of the kit according to the present invention were each daubed three times, alternately, one layer at a time, on a 0.8 cm×0.8 cm slide surface (compounded by 450 IU thrombin and 40 mmol/L CaCh).

(2) The layers of the high concentrated fibrinogen solution and thrombin solution were air dried and inspected with an electron microscope.

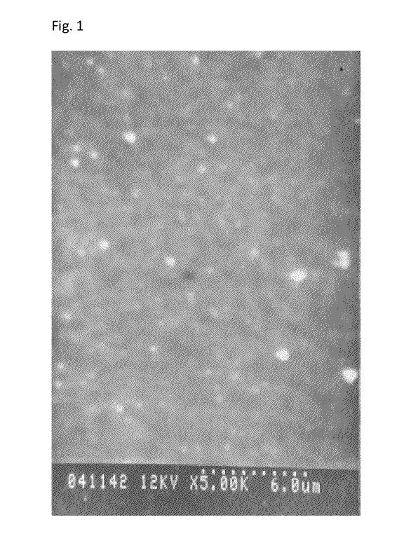

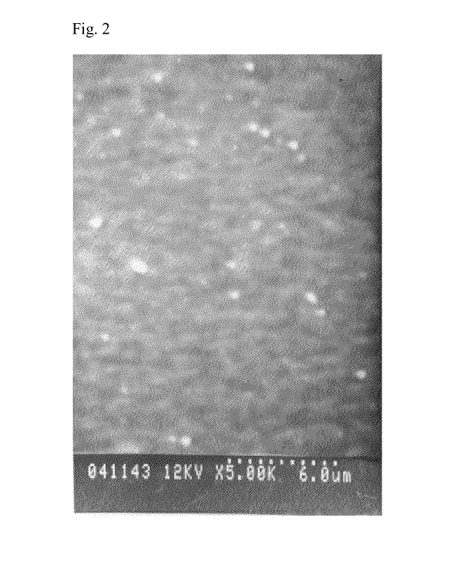

FIG. 1 and FIG. 2 are electron microscope photographs of the dried high concentrated high concentrated fibrinogen and thrombin layers. The amplification is 5000.

From these photographs, the smooth glue membrane surface can be seen. There are no distinct holes in the glue membrane. It can be induced from the amplified scale that the mesh bore diameter is 0.6 μm or less, as little as 0.0 μm. Human cancer tumor cell size is 10-100 μm. Thus, the glue membrane compounded by human plasma high concentrated fibrinogen and thrombin can HOLD BACK human TUMOR CELLS and PREVENT PERVASION.

### Example 2

From a glue membrane kit according to the present invention for preventing dissociative tumor cell pervasion:

(1) A thin and smooth glue membrane was produced on the bottom surfaces of the cell culture inserts that are placed into the wells of the tissue culture plate of a cell invasion assay kit ECM550, commercially available from Chemicon International of Temecula, Calif., to form a coating thereon by using the method of EXAMPLE 1. Then, a cell invasion experiment was carried out according to the instructions provided in the cell invasion assay kit. The invasive cells in the cell suspensions that were placed in the inserts included carcinoma ventriculi cell lines (tumor of the stomach), human gastric adenocarcinoma cell line KN45, and AGS human cultured gastric adenocarcinoma cells from Ruijing Hospital of Shanghai, China, as well as human breast cancer cells MDA-MB-231 and colon cancer cells Ls 174T from SBI (System Biosciences of Mountain View, Calif.).

(2) In accordance with the instructions in the cell invasion assay kit, the spent medium was discarded, the inner membrane was cleared using a cotton-tipped swab, and the inserts were stained for 20 minutes and air dried. There was no high concentrated fibrinogen or thrombin in the control wells of the tissue culture plate.

See FIGS. 1 and 2.

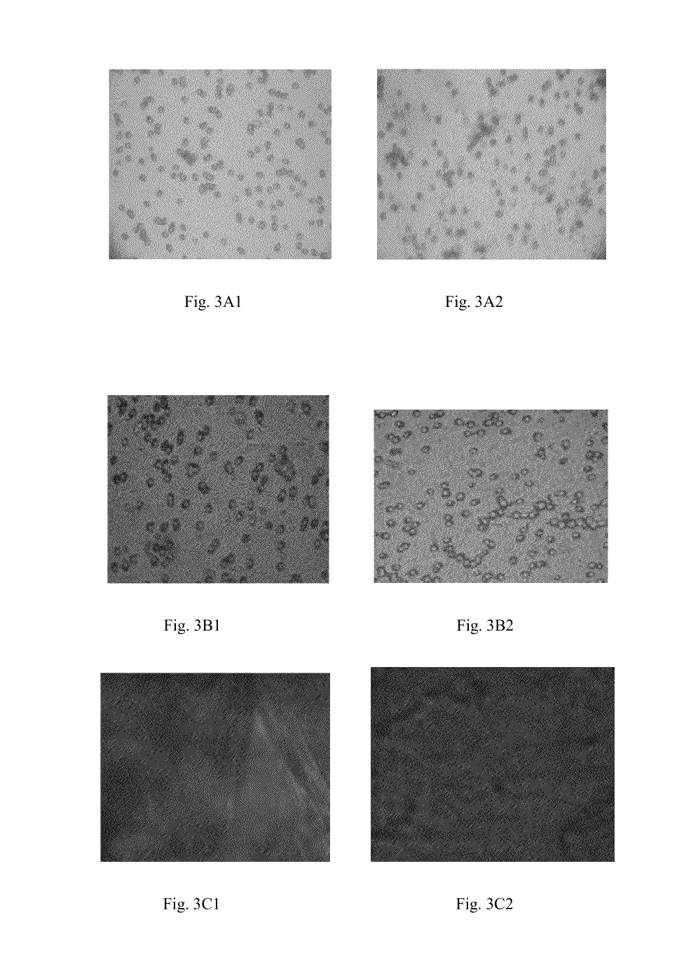

(3) Electron microscope photographs were taken, including the electron microscope photographs of FIG. 3A1, FIG. 3A2, FIG. 3B1, FIG. 3B2, FIG. 3C1 and FIG. 3C2, and records were made.

FIG. 3A1, FIG. 3A2, FIG. 3B1, FIG. 3B2, FIG. 3C1 and FIG. 3C2 show that the glue membrane of EXAMPLE 2 arrested dissociative tumor cells in the inserts and prevented tumor cell pervasion through the glue membrane and, therefore, also support the conclusion that the glue membrane can hold back human tumor cells and prevent pervasion.

FIGS. 3A1 and 3A2 are electron microscope photographs without a glue membrane showing that cell invasion occurs where there is no glue membrane.

FIGS. 3B1 and 3B2 are photographs of prevention of cell invasion on the inner side of the glue membrane. FIGS. 3C1 and 3C2 are photographs of the prevention of cell invasion on the exterior side of the glue membrane. It is very clear that glue membrane compounded by human plasma high concentrated fibrinogen and thrombin does hold back human tumor cells and prevent pervasion.

It will further be appreciated by those skilled in the art and it is contemplated that variations to the embodiments illustrated and described herein may be made without departing from the spirit and scope of the present invention.

Accordingly, it is intended that the foregoing description is illustrative only, and the true spirit and scope of the invention will be determined by the appended claims.

See FIGS. 3A1, 3A2, 3B1, 3B2, 3C1 and 3C2.

See FIGS. 3D1, 3D2, 3D3, 3D4, 3D5, 3D6, 3D7,

Increased heat temperature intensify the density of the Glue membrane Various fold magnification has shown that we can't see any pore of FS gel membrane when is magnified at 3810 and 1600. At 27500 the pore of gel membrane is 0.005 micrometers and the heating temperature is at 101° C.

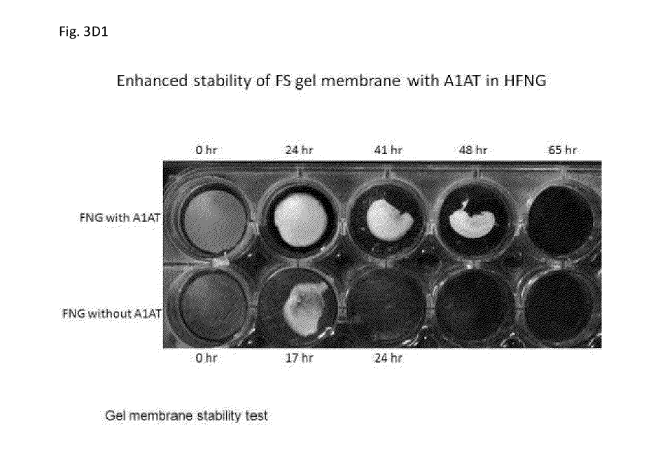

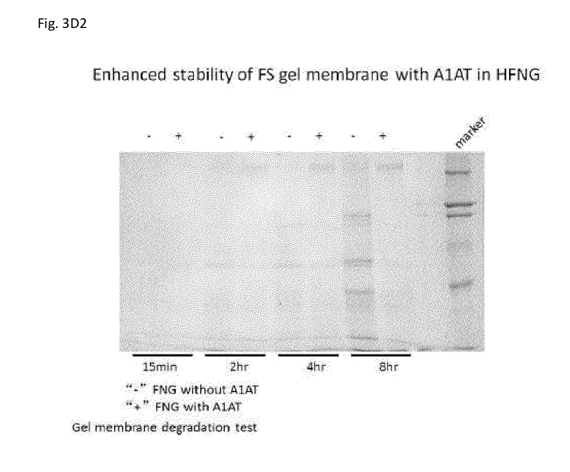

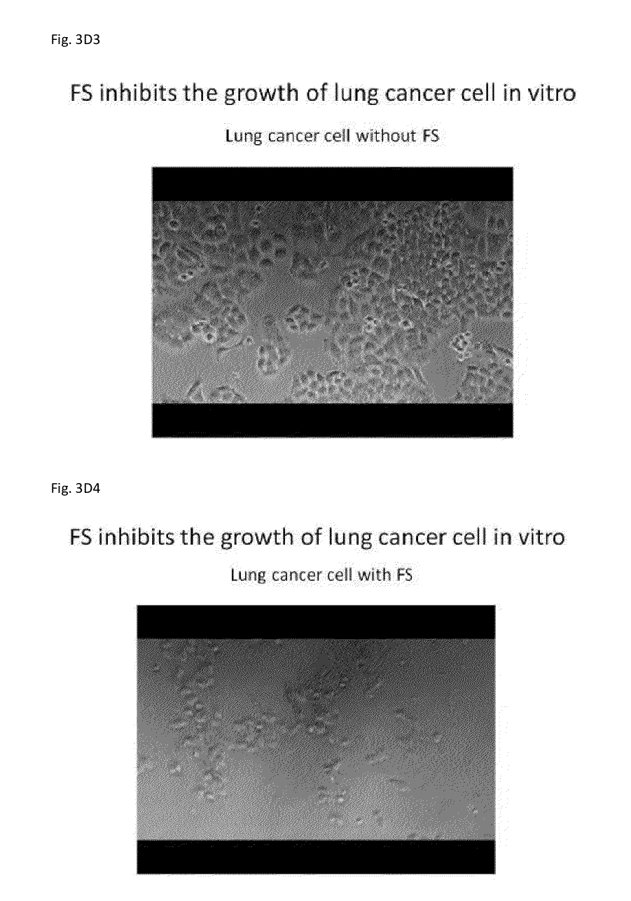

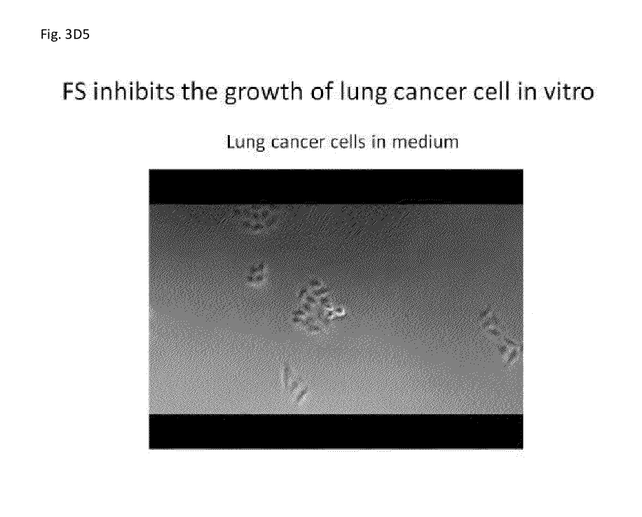

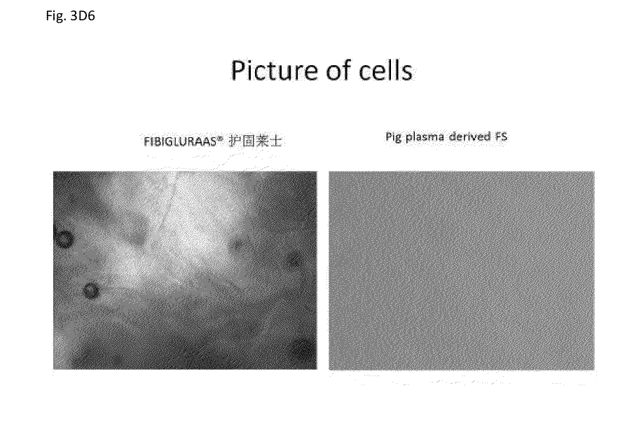

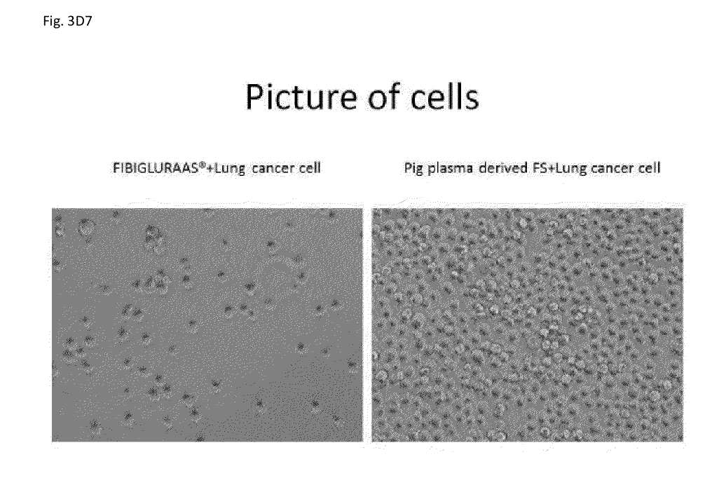

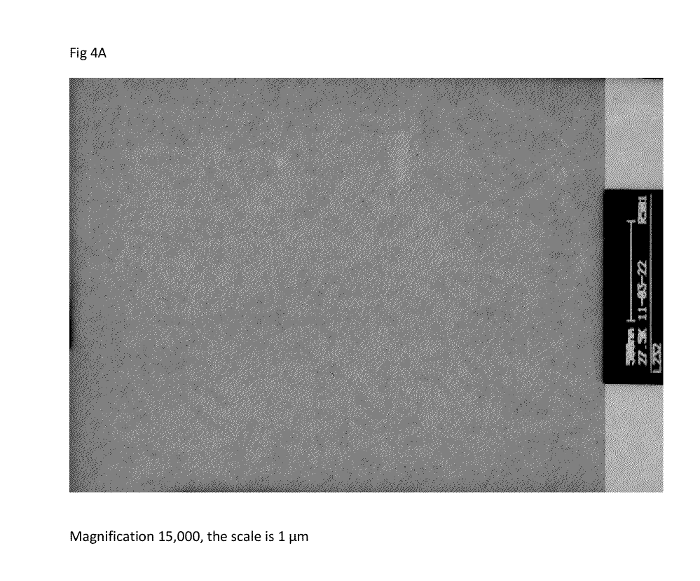

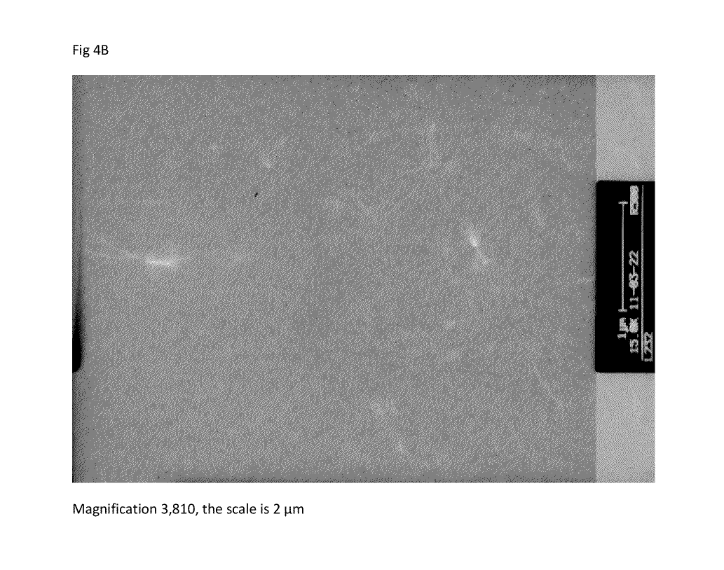

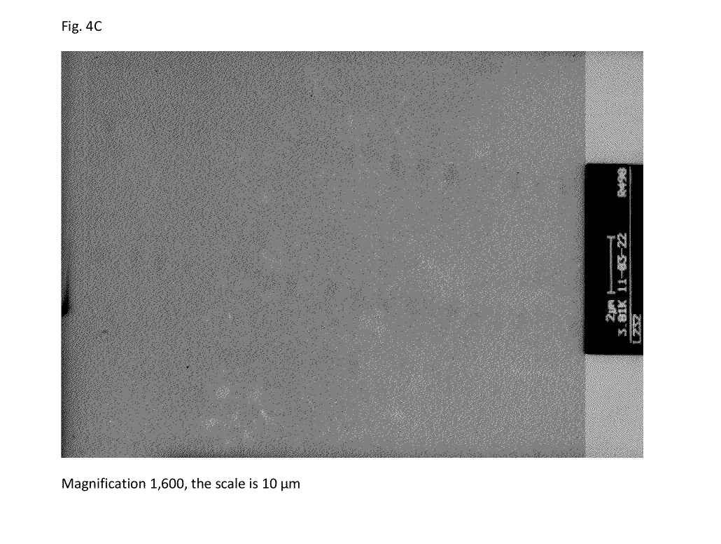

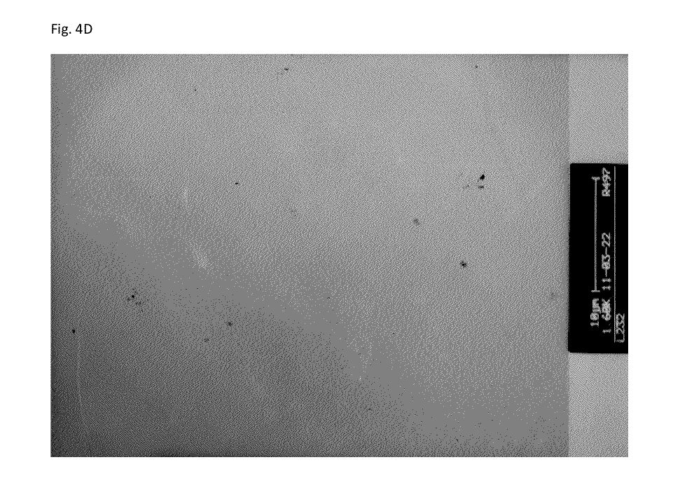

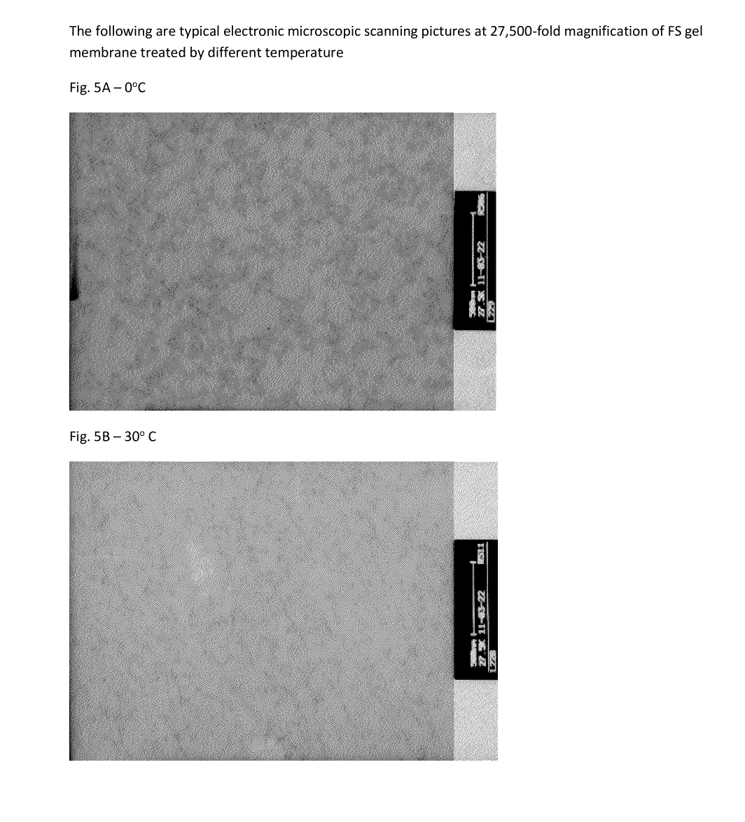

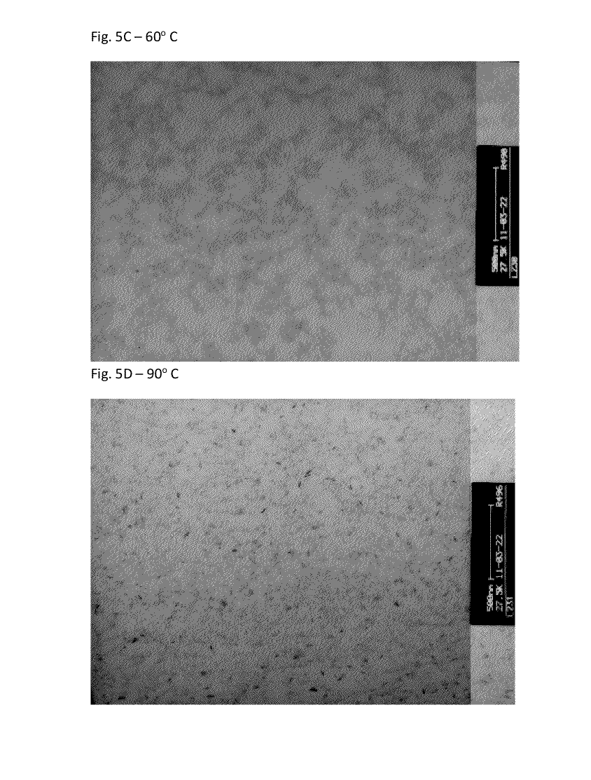

Electronic microscopic scanning results:
The pictures are taken under electronic microscopic with escalated magnification (up to 27,500-fold magnification). The corresponding scales under various magnifications are also marked. Basically we can't see any pore of FS gel membrane when is magnified at 3,810 and 1,600. At magnification of 15,000, we can see the pores of gel membrane about 0.1-0.2 μm. At 27,500 magnification, we can see the pores of gel membrane less than 0.1 μm in its diameter.
Magnification 27,500, the scale is 0.005 μm in FIG. 4A.
Magnification 15,000, the scale is 1 μm in FIG. 4B.
Magnification 3,810, the scale is 2 μm in FIG. 4C.
Magnification 1,600, the scale is 10 μm in FIG. 4D.
FIGS. 5A-5E are typical electronic microscopic scanning pictures at 27,500-fold magnification of FS gel membrane treated by different temperature. FIG. 5A is at 0° C. FIG. SB is at 30° C. FIG. 5C is at 60° C. FIG. 5D is at 90° C. FIG. 5E is at 101° C.

A1AT can be detected in the high concentrated fibrinogen.
1. The existence of A1AT in the high concentrated fibrinogen can improve the stability of FS Glue membrane.
2. Neutralization of A1AT will accelerate the degradation of FS Glue membrane.
3. The further enrichment of A1AT in the high concentrated fibrinogen can greatly improve the stability of FS Glue membrane.

### Examples

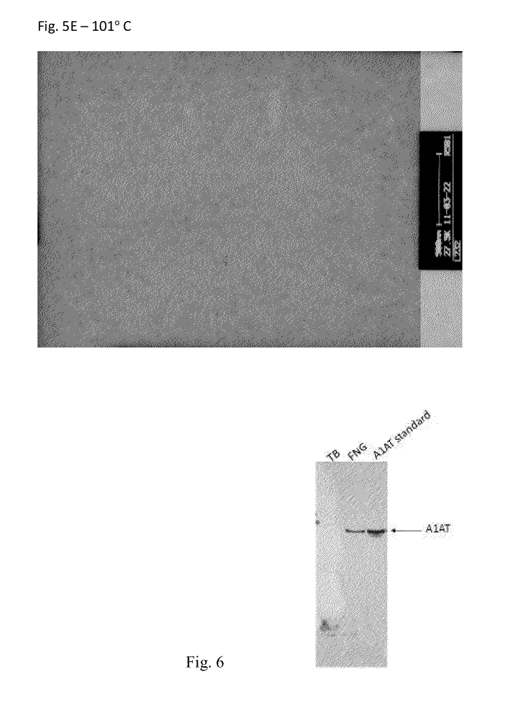

1. In FIG. 6, A1AT is enriched in high concentrated fibrinogen. The A1AT protein was detected by western blot using a polyclonal antibody against human A1AT. “TB” and “FNG” stand for the thrombin and high concentrated fibrinogen in Fibrinogen®. TB was used for a negative control. “A1AT standard” stands for a commercially available A1AT as a positive control.

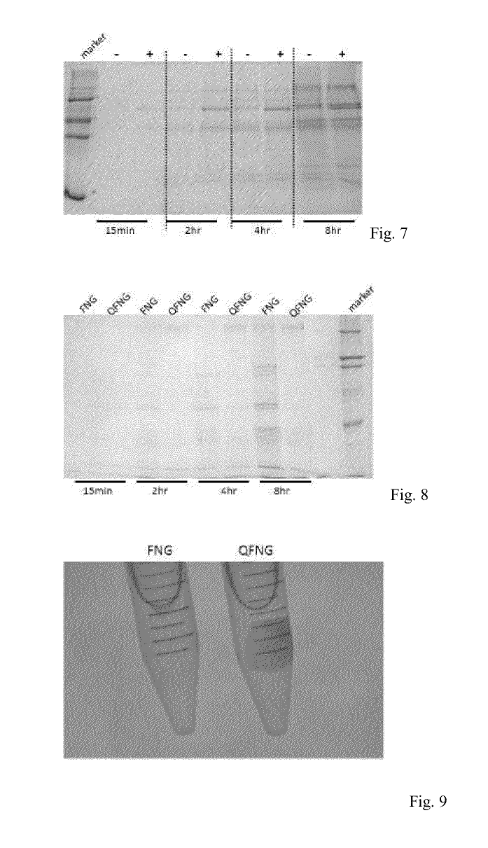

As can be seen from FIG. 7, the degradation of an FS glue membrane accelerated when A1AT in FNG is neutralized by an A1AT antibody. An A1AT polyclonal antibody was added to the high concentrated fibrinogen of Fibrinogen®. “−” represents Fribinogen®+WFI, which means A1AT will keep its activity. “+” represents Fribinogen®+anti-A1AT antibody, which means A1AT is neutralized. The degradation of FS glue membranes is detected using SDS-PAGE (sodium dodecyl sulfate). When the A1AT is neutralized, the degradation of FS glue membranes begins as early as 15 minutes after the glue membrane is formed. The difference still exists at 2 hours, 4 hours, and 8 hours after the glue membrane is formed.

FIG. 8 shows that enrichment of A1AT in the purification process of high concentrated fibrinogen makes FS glue membrane much more stable. The degradation of FS glue membrane is compared between fibrinogen (FNG) and A1AT enriched high concentrated fibrinogen (QFNG). The degradation of FS glue membrane with A1AT enriched FNG will not begin within the first 8 hours after the gel is formed, which means it is much more stable.

FIG. 9 shows that the FS glue membrane can stay in a stable status as long as 48-72 hours using A1AT-enriched high concentrated fibrinogen. FIG. 9 shows the conditions of the membranes 48 hours after they are formed. The glue membrane is still in a stable status when using A1AT-enriched high concentrated fibrinogen (QFNG). However, the glue membrane of FNG has already degraded.

In Vitro Preliminary Study:

In vitro studies of the Thrombin High Concentrate Fibrinogen and AFOD (HDL Apoa1) proteins containing good healthy cells inhibit all the solid tumor cancer cells.
Gastric cancer cell AGS in In Vitro Studies we found Thrombin a protein containing
KH good healthy cells, high concentrate Fibrinogen another protein containing
KH good healthy cells in combination of AFOD (HDL ApoAl) another protein with KH good healthy cells inhibit cancer cells in the surface areas where solid tumors have been removed (in reference to another patent application): application number 611457,380 filed on 03114/2011.
The following nine cancer cell lines:
1. Gastric cell line (AGS)
2. Cervical cancer cell line HELA
3. Breast cancer cell line SK-BR-3
4. Ovarian cancer cell line SK-OV-3
5. Lung adenocarcinoma cell line SPC-A-1
6. Esophageal cancer cell line TE-I
7. Liver cancer cell line BEL-7402
8. Pancreas cancer cell line PANC-1
9. Leukemia cancer cell line DAMI
have been tested with Thrombin/High concentrated Fibrinogen and AFOD (HDL ApoAl) in preliminary in vitro Studies at our R&D Lab and has found that cancer cells have been inhibited at certain level of protein concentration.

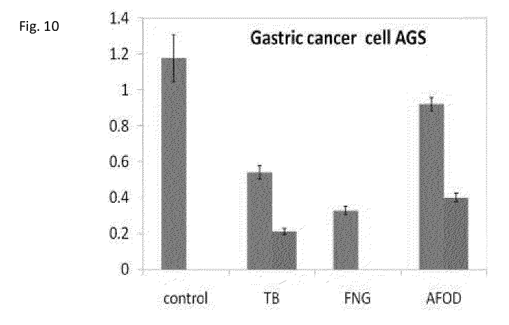

### See FIG. 10.

In the table above the yellow color is the control medium with 0% of protein to compare with TB (Thrombin), FNG (Fibrinogen) and AFOD (HDL Apoal)

A. TB (Thrombin)

### 1. Gastric Cell Line (AGS)

See FIG. 11. Also see FIGS. 12A, 12B and 12C for Control, TB 5 U/mL and TB 25 U/mL, respectively.

### 2. Cervical Cancer Cell Line HELA

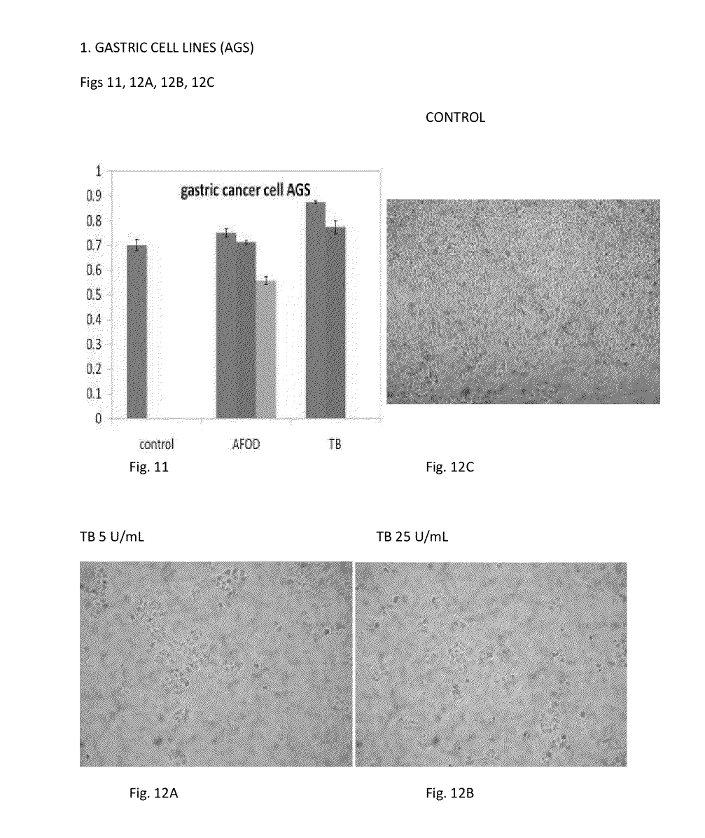

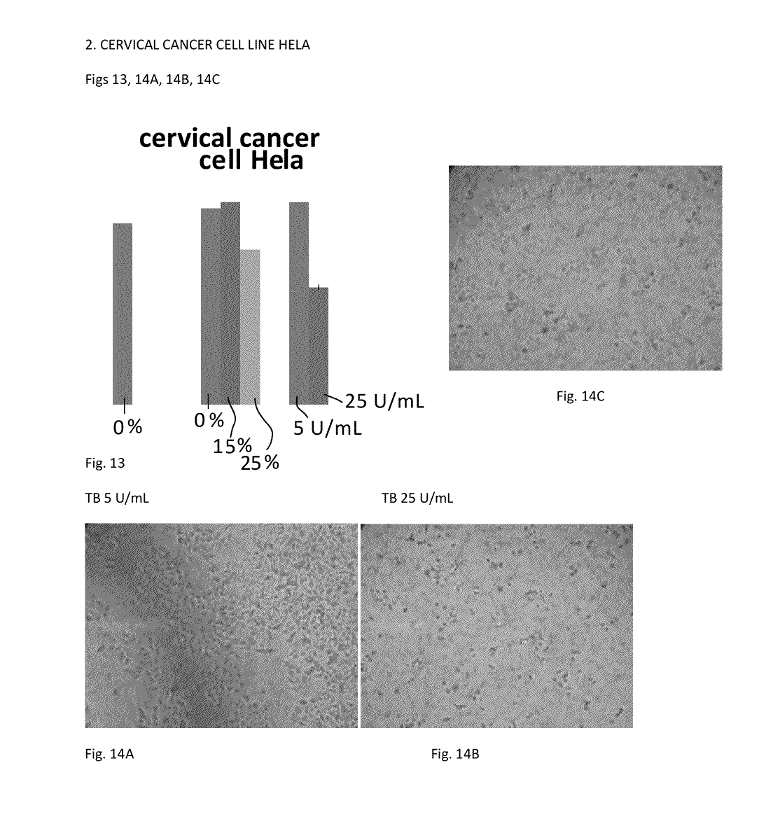

See FIG. 13. Also see FIGS. 14A, 14B and 14C for Control, TB 5 U/mL and TB 25 U/mL, respectively.

### 3. BRE Breast Cancer Cell Line SK-BR-3

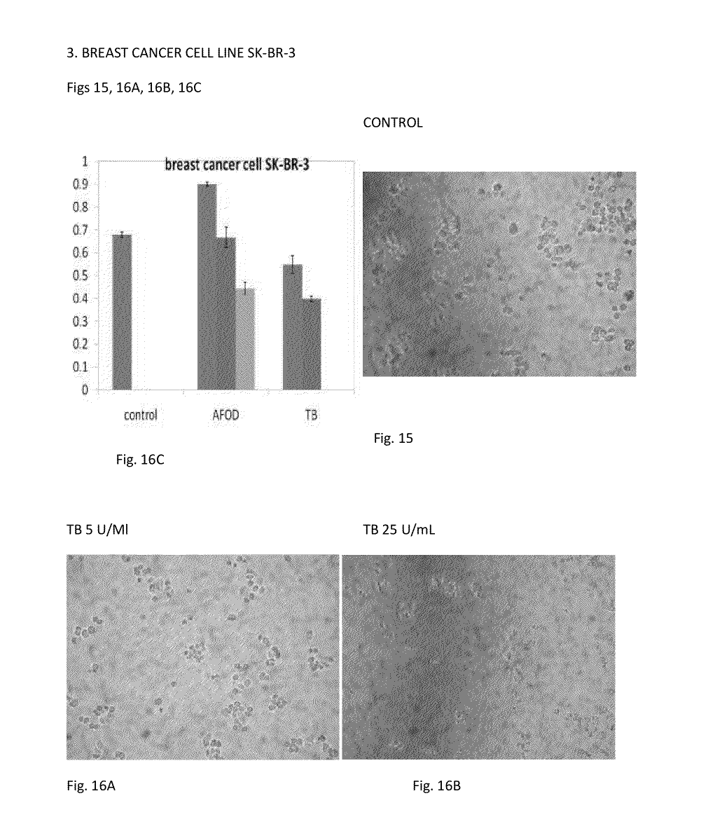

See FIG. 15. Also see FIGS. 16A, 16B and 16C for Control, TB 5 U/mL and TB 25 U/mL, respectively.

### 4. Ovarian Cancer Cell Line SK-OV-3

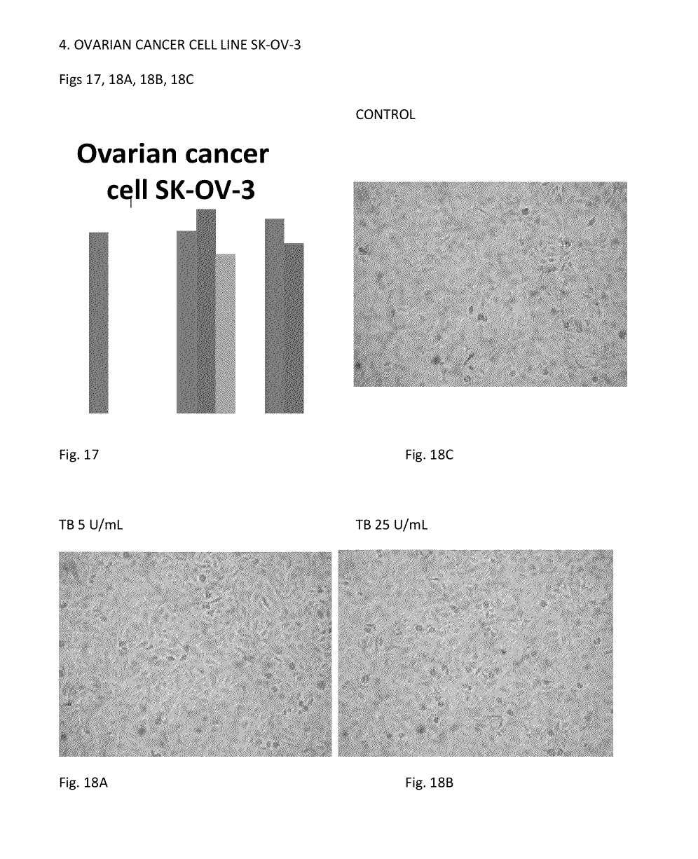

See FIG. 17. Also see FIGS. 18A, 18B and 18C for Control, TB 5 U/mL and TB 25 U/mL, respectively.

### 5. Lung Adenocarcinoma Cell Line SPC-A-1

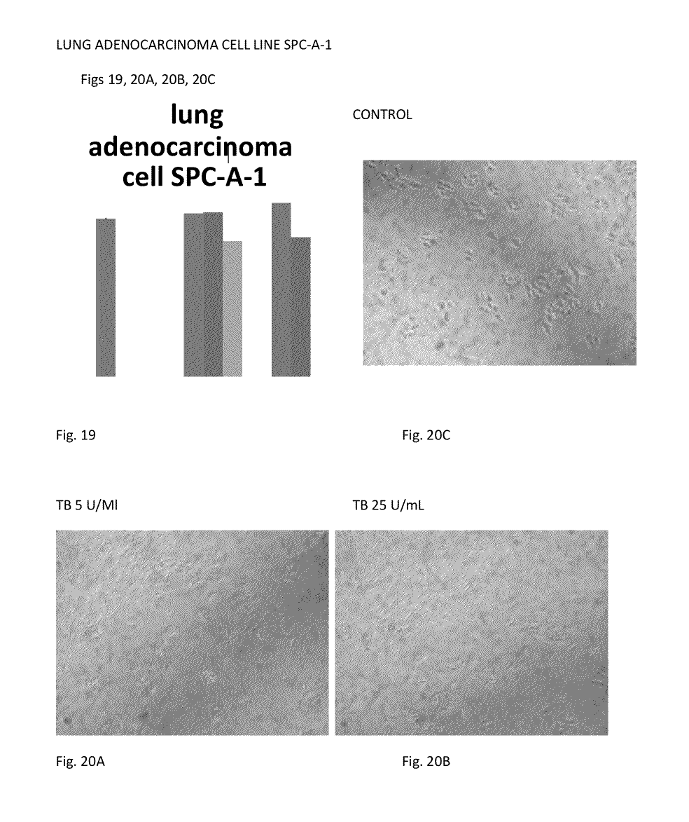

See FIG. 19. Also see FIGS. 20A, 20B and 20C for Control, TB 5 U/mL and TB 25 U/mL, respectively.

### 6. Esophageal Cancer Cell Line TE-I

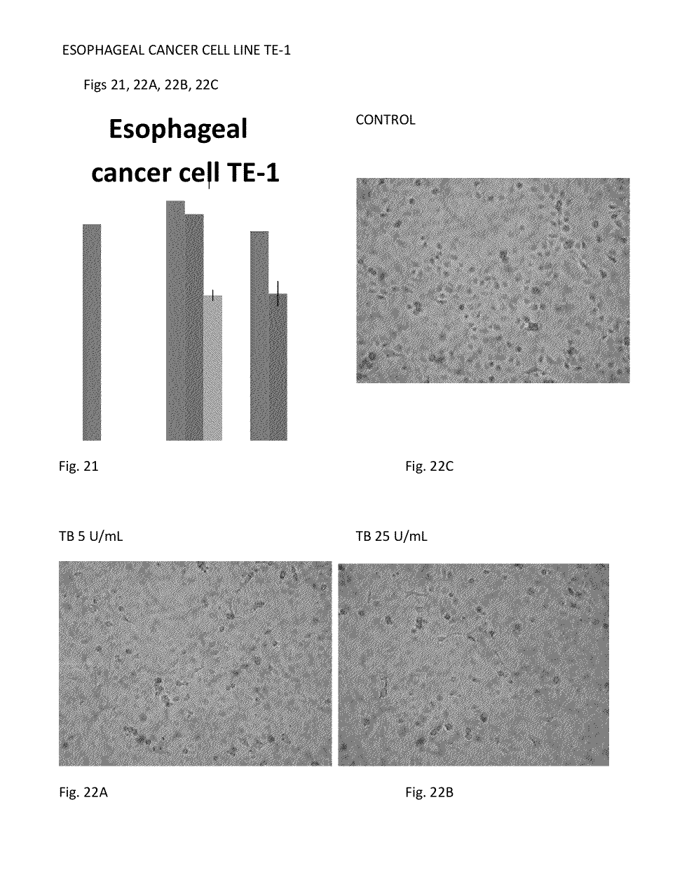

See FIG. 21. Also see FIGS. 22A, 22B and 22C for Control, TB 5 U/mL and TB 25 U/mL, respectively.

### 7. Liver Cancer Cell Line BEL-7402

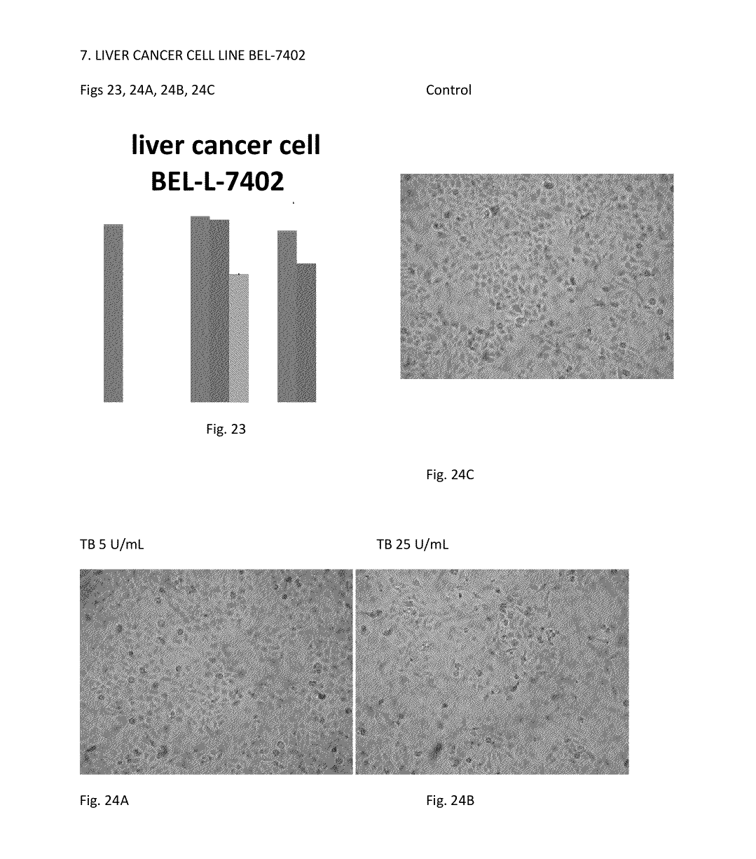

See FIG. 23. Also see FIGS. 24A, 24B and 24C for Control, TB 5 U/mL and TB 25 U/mL, respectively.

### 8. Pancreas Cancer Cell Line PANC-1

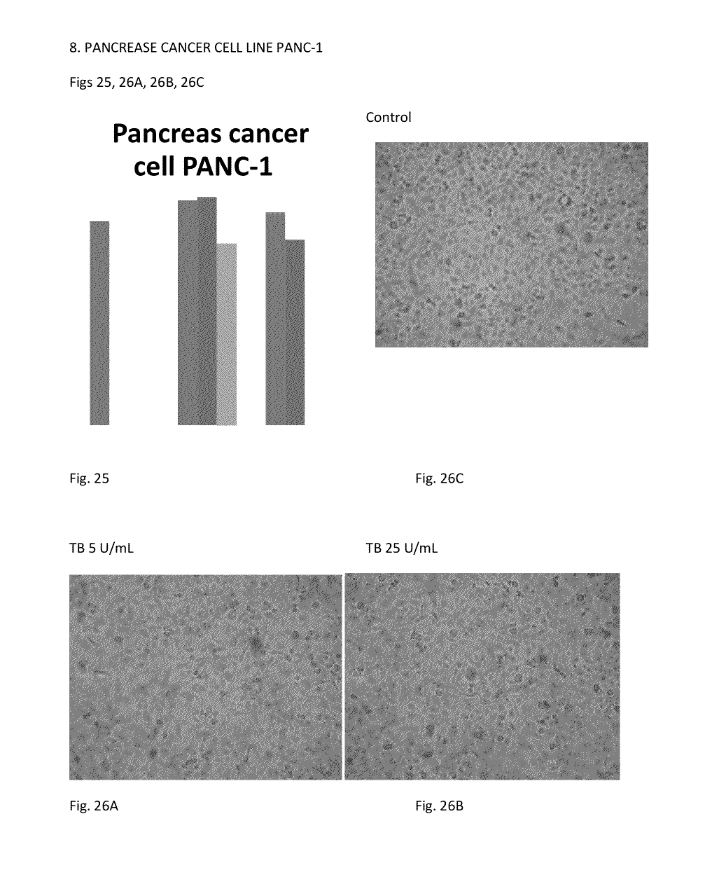

See FIG. 25. Also see FIGS. 26A, 26B and 26C for Control, TB 5 U/mL and TB 25 U/mL, respectively.

### 9. Leukemia Cancer Cell Line DAMI

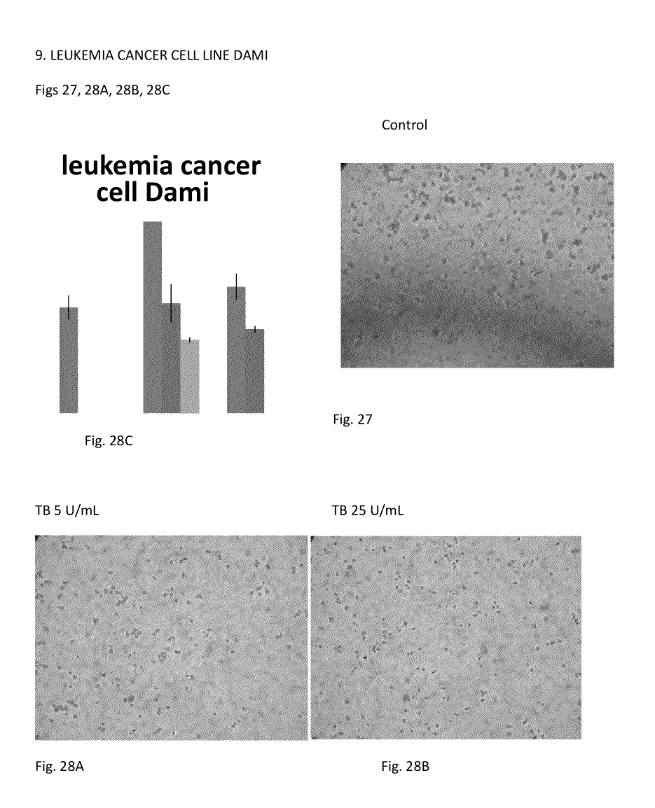

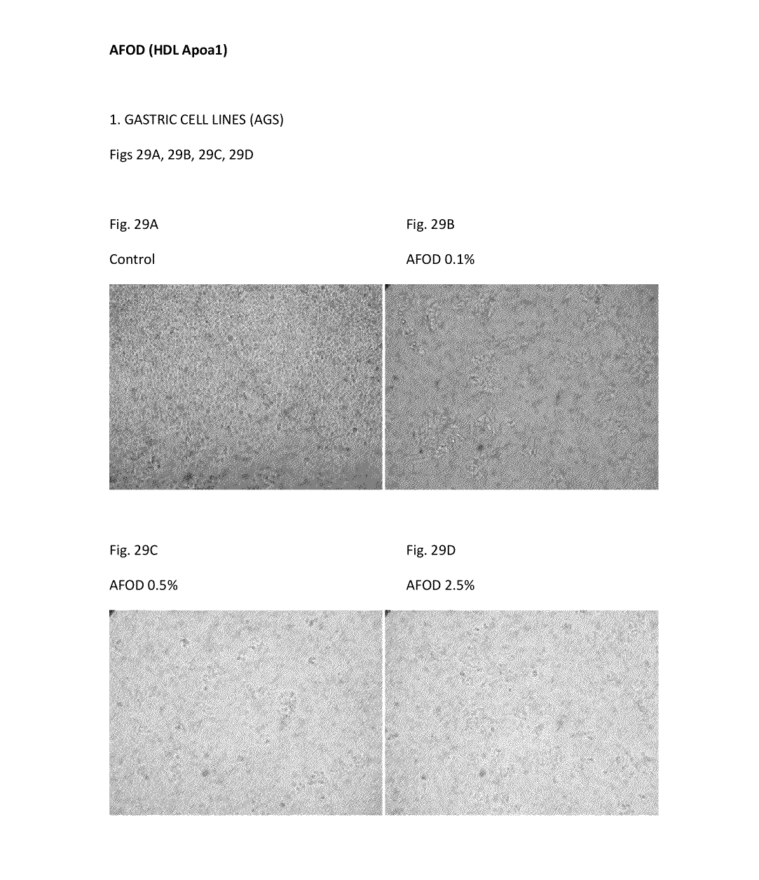

See FIG. 27. Also see FIGS. 28A, 28B and 28C for Control, TB 5 U/mL and TB 25 U/mL, respectively.

B. AFOD (HDL Apoal)

### 1. Gastric Cell Lines (AGS)

See FIGS. 29A, 29B, 29C and 29D for Control, AFOD 0.1%, AFOD 0.5% and AFOD 2.5%, respectively.

### 2. Cervical Cancer Cell Line HELA

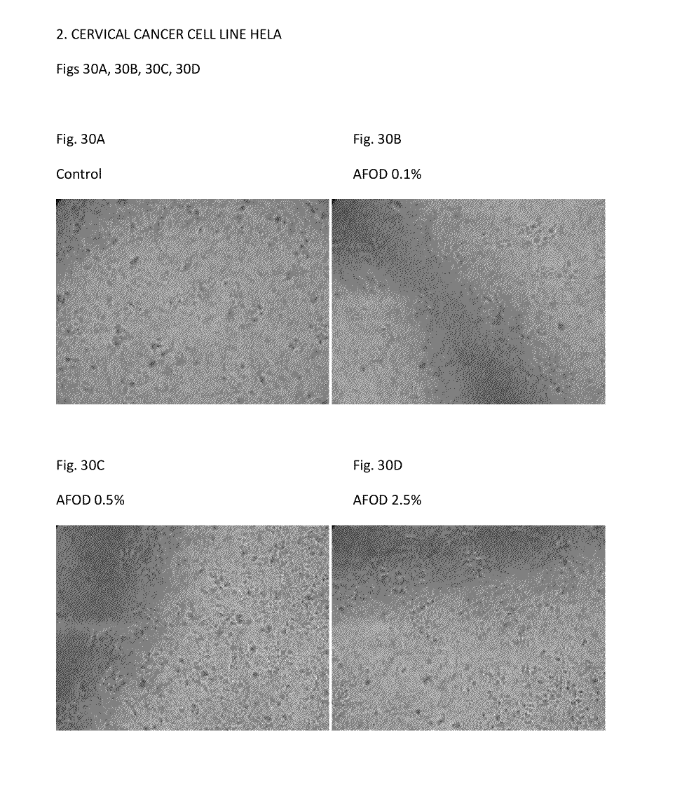

See FIGS. 30A, 30B, 30C and 30D for Control, AFOD 0.1%, AFOD 0.5% and AFOD 2.5%, respectively.

### 3. Breast Cancer Cell Line SK-BR-3

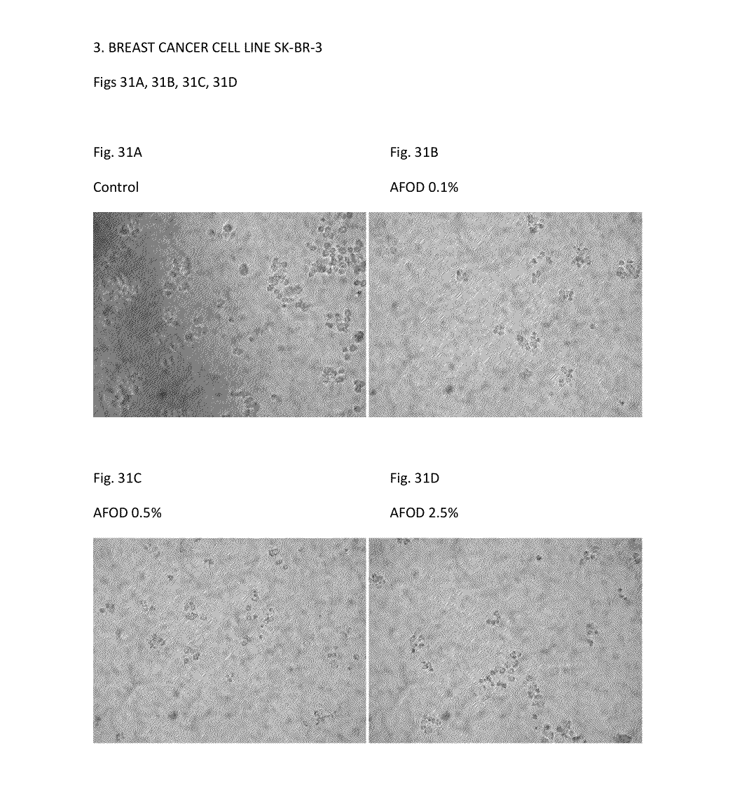

See FIGS. 31A, 31B, 31C and 31D for Control, AFOD 0.1%, AFOD 0.5% and AFOD 2.5%, respectively.

### 4. Ovarian Cancer Cell Line SK-OV-3

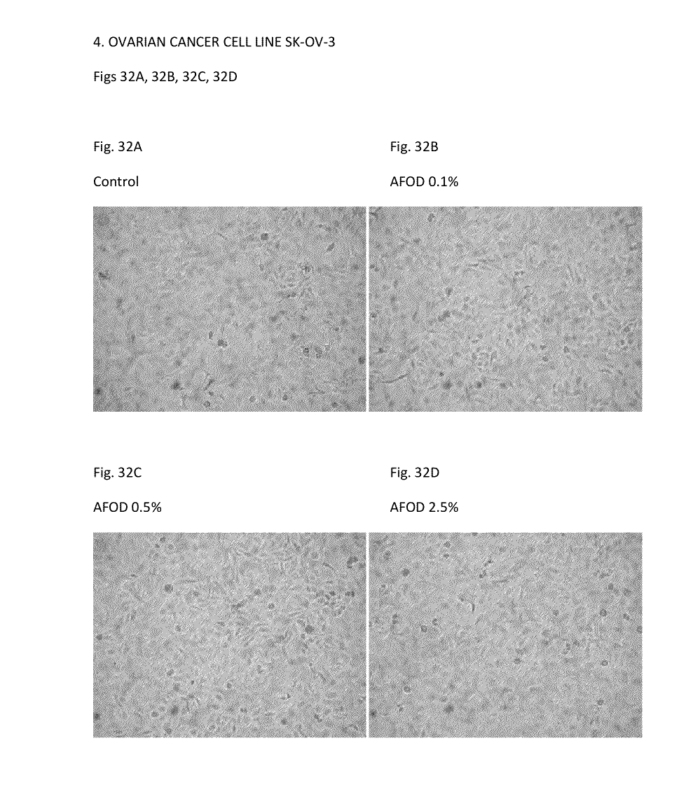

See FIGS. 32A, 32B, 32C and 32D for Control, AFOD 0.1%, AFOD 0.5% and AFOD 2.5%, respectively.

### 5. Lung Adenocarcinoma Cell Line SPC-A-1

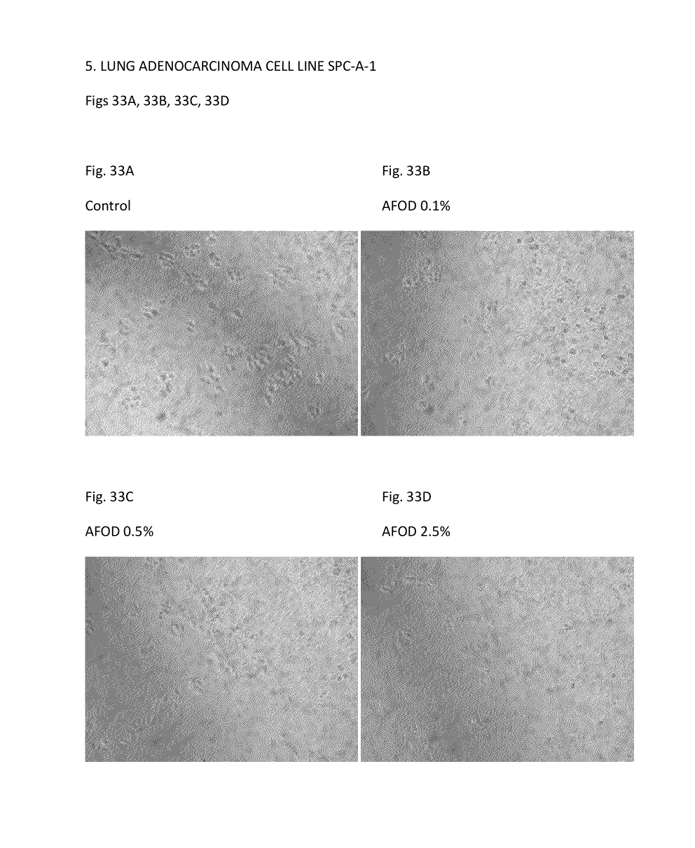

See FIGS. 33A, 33B, 33C and 33D for Control, AFOD 0.1%, AFOD 0.5% and AFOD 2.5%, respectively.

### 6. Esophageal Cancer Cell Line TE-I

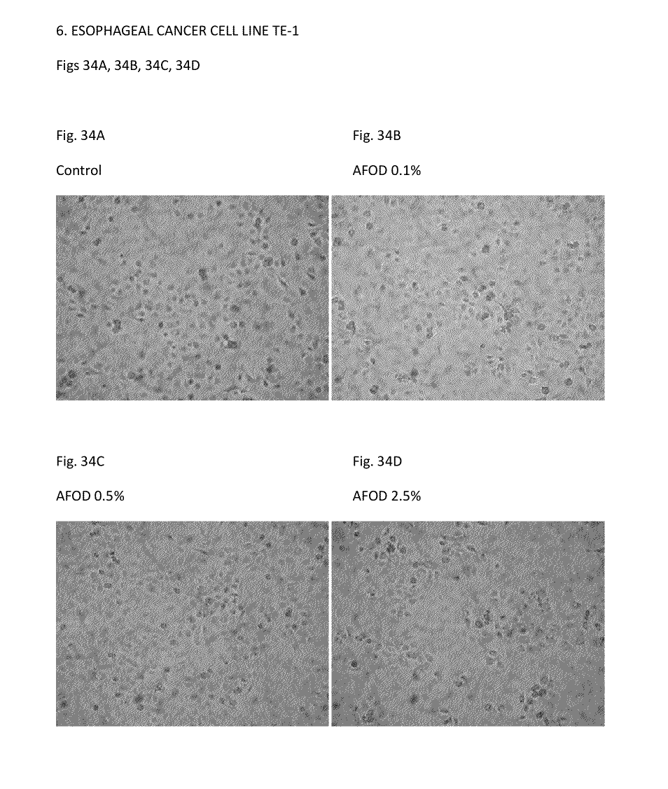

See FIGS. 34A, 34B, 34C and 34D for Control, AFOD 0.1%, AFOD 0.5% and AFOD 2.5%, respectively.

### 7. Liver Cancer Cell Line BEL-7402

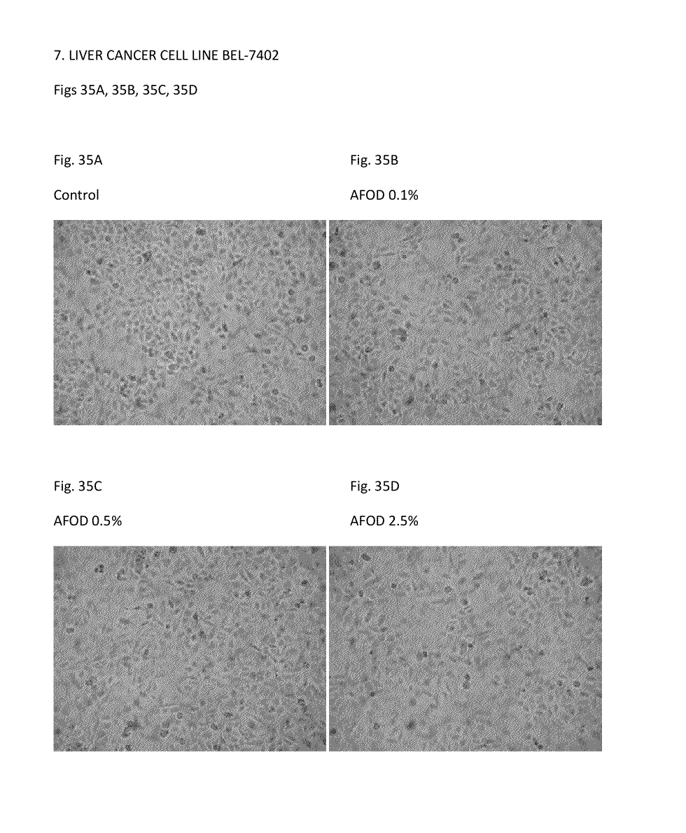

See FIGS. 35A, 35B, 35C and 35D for Control, AFOD 0.1%, AFOD 0.5% and AFOD 2.5%, respectively.

### 8. Pancreas Cancer Cell Line PANC-1

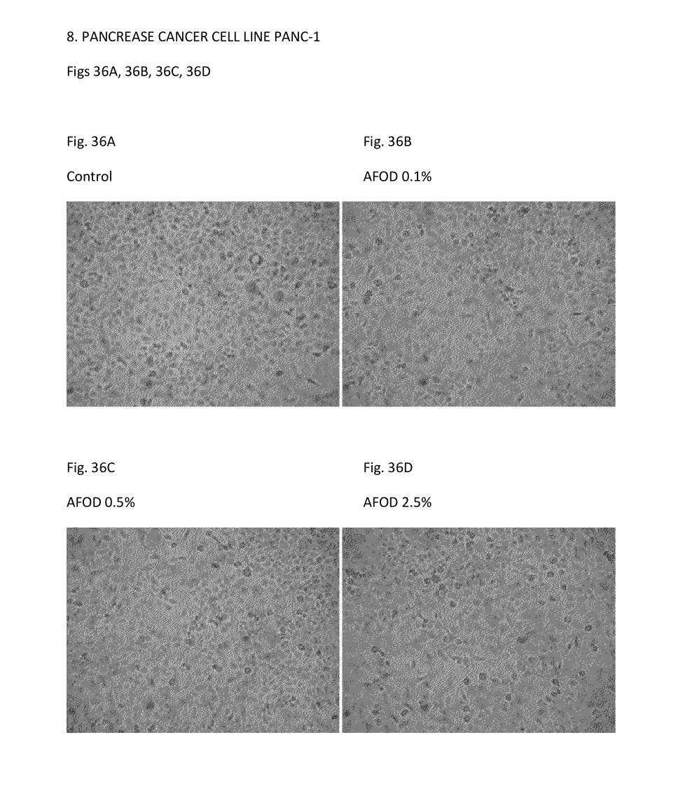

See FIGS. 36A, 36B, 36C and 36D for Control, AFOD 0.1%, AFOD 0.5% and AFOD 2.5%, respectively.

### 9. Leukemia Cancer Cell Line DAMI

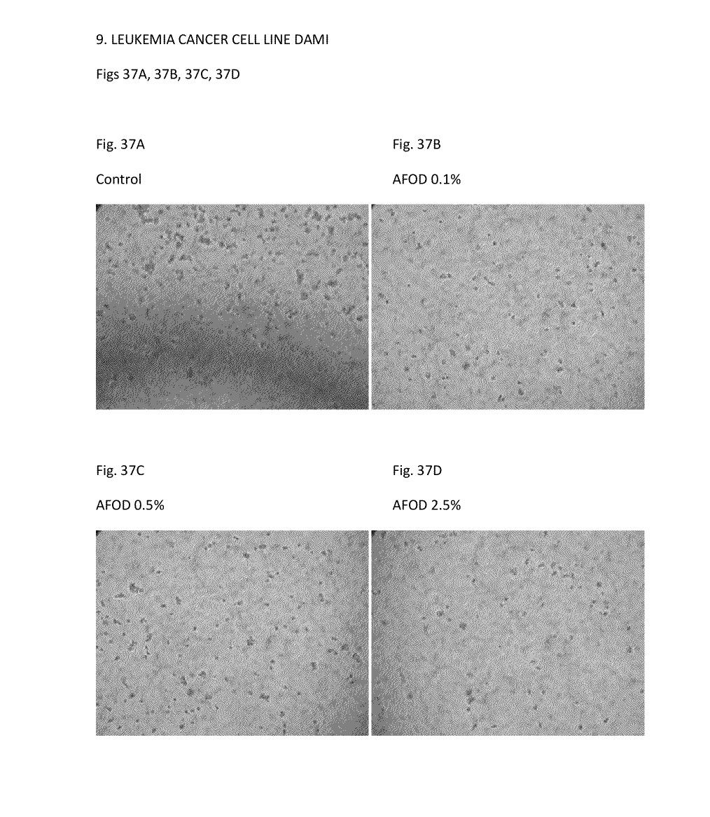

See FIGS. 37A, 37B, 37C and 37D for Control, AFOD 0.1%, AFOD 0.5% and AFOD 2.5%, respectively.

### Further In Vitro Studies:

In this study we want to demonstrate that in addition to the above nine cell lines we have added more different cells line at 0%, 2% and highest concentration of 10% in order to completely inhibit all cancer cells.

In Vivo STUDY:

Treated groups, which showed that fibrin sealant had no side effects Fibrin Sealant plus a Slow-released RAAS 1 to 45-FU Agent as a Prophylaxis for Peritoneal Dissemination of Gastric Cancer in Nude Mice Model

Objectives

To evaluate the efficacy of Fibrin Sealant, used alone or combined with a slow-released RAAS 1 to 45 FU agent as an adjuvant instrument to prevent cancer cells from becoming detached and spreading into the abdominal cavity during the operation of gastrointestinal cancer.

Background

Gastrointestinal cancer still remains the most frequent malignancy worldwide. As far as the adeno-carcinoma of the stomach is concerned, it was the leading cause of cancer-related death worldwide through most of the twentieth century, and continues to be responsible for the majority of cancer deaths in developing countries. An estimated 875,000 new cases are diagnosed annually worldwide, not to count the other various cancer.

Official data from the government of China indicate that there are up to now 479 cancer villages in China. In China every minute five people die because of cancer and six people are diagnosed with cancer, particularly in Shanghai official government data show that one out of fifty women in Shanghai has cancer.

Although combined therapies, including chemotherapy, radiation therapy and immunotherapy are performed in addition to surgical radical resection, nearly 50% of patients still die of recurrence, and a major form of recurrence was peritoneal dissemination. Concomitantly, peritoneal dissemination has been regarded as an important prognostic factor in GI cancer. It has also been indicated that increased peritoneal dissemination could result from the liberation, seeding and dissemination of cells from the primary tumor due to the surgical manipulation and mechanical tumor spillage. Moreover, the dissected and rough peritoneal surface may served as an appropriate “soil” for those iatrogenic tumor seeds. Besides strictly following the “non-touch” principle during the operation, it is absolutely necessary to develop new techniques and adjuvant equipment to abate unnecessary “nosocomial” tumor dissemination.

Shanghai RAAS Blood Products Company has recently observed that the glue membrane formed by lyophilized or frozen thrombin and lyophilized or frozen high concentrated fibrinogen (Fibrin Sealant Fribinogen) can successfully prevent highly aggressive solid tumor cell lines, such as gastric cancer cell lines MKN45 and AGS, colon cancer cell line LoVo, as well as breast cancer cell line MCF-7, from passing through. The already finished in vitro study demonstrated the efficacy and potential role of FS as an adjuvant strategy in preventing solid tumor dissemination with the mechanism of creating a compact mechanical barrier.

Under electro-microscopy, the pore of glue membrane formed by Fibrin Sealant was measured to be 0.6 μm or less in diameter, as little as 0.0 μm, while the size of solid tumor cell ranges from 10 to 100 μm. Theoretically, the dense and well-intertextured membrane formed by Fibrin Sealant can provide an optimal mechanical barrier to prevent the invasion and penetrating of tumor cells. As shown in FIG. 1, by using cell invasion assay (ECM550 kit, Chemicon International), it was found that the glue membrane formed by the Fibrin Sealant did prevent the above-mentioned high-aggressive tumor cell lines pervasion.

Besides such an exciting in vitro finding, the results of an in vivo study has also demonstrated that FS application can successfully prevent tumor cell dissemination intraperitoneally. A nude mice model was established to evaluate such efficacy.

Nude mice underwent laparotomy after anesthetization. The peritoneal surface was destroyed into multi-spots by using forceps, so as to create a congenial environment (“soil”) for the “seeding” of cancer cells. Tumor cells were sprayed into the abdominal cavity of mice in each group. Subsequently, Fibrin Sealant was applied to cover the peritoneal surface in the fibrin sealant group, while the control group were treated by normal saline only. After the glue membrane formed completely, the peritoneal cavity was closed. Two weeks after tumor cell implantation, peritoneal dissemination was identified macroscopically as small nodules growth on the surface of the small intestine and peritoneum, as well as the mesentery. A significantly larger number of peritoneal dissemination nodules were observed in the normal saline treatment group than in the FS treatment group. Furthermore, in the mice of self-control design, two weeks after tumor cells implantation, significantly larger and fused peritoneal cancer nodules were observed in the normal saline treatment side than in the Fibrin Sealant treatment side. And the above-mentioned in vivo data have been submitted for another international patent.

Herein, another idea has merged: whether or not the combination of FS with chemotherapeutic agent might reach a synergistic efficacy in preventing tumor cell dissemination.

Methodology (Study Design and Animal Models Development)

1. Preparation of Tumor Cells

Human gastric adenocarcinoma cells (cell line MKN45), were harvested from sub-confluent cultures (80-90% confluent) by a brief exposure to 1% trypsin. Trypsinization was stopped with a culture medium containing 10% fetal bovine serum, and the cells were washed twice by PBS and re-suspended in a serum-free medium. Only suspensions consisting of single cells with 90% viability were used for the experiments. The amounts of tumor cells for intra-peritoneal implantation were:

TABLE 1
Implanted tumor cells and amounts

| | Name of | Site of | Implanted |
|---|---|---|---|
| Cancer type | cell line | implantation | amounts |
| Stomach cancer | MKN45 | i.p. | 1*10⁷ per mouse |

These cells were in 0.2 ml serum-free DMEM medium for peritoneal surface coating.

2. Animals

Female Athymic nu/nu nude mice (NCI), at 6-7 weeks of age, weight −20 g, were purchased from the Shanghai Experimental Animal Center (License No.SYXK(Shanghai) 2003-0026), a Chinese animal facility with an international license. The mice were housed and maintained in a laminar airflow cabinet under specific pathogen-free conditions, and were housed for at least 7 days before use. All facilities used in this protocol were approved by the Animal Care and Use Committee of Shanghai Jiao-tong University.

3. Establishment of the Peritoneal Tumor Implanted Mouse Model

The mice were anesthetized with diethyl ether, and a simple laparotomy was performed via a central incision. The experiments were performed according to the following designs:

The mice were randomized classified into the following 4 groups: (1) FS group, (2) RAAS 1 to 45 FU group, (3) FS plus RAAS 1 to 45 FU group, and (4) control group. Mice in these 4 groups were underwent laparotomy after anesthetization. The peritoneal surface was destroyed into multi-spots by using forceps, so as to create a congenial environment (“soil”) for the “seeding” of cancer cells. Tumor cells were sprayed into the abdominal cavity of mice in each group. Subsequently, Fibrin Sealant, RAAS 1 to 45 FU, or both agents were applied to cover the peritoneal surface in FS group mice, RAAS 1 to 45 FU group mice, and combination group mice, respectively, while the control group were treated by normal saline only. After the glue membrane formed completely, the peritoneal cavity was closed.

4. Drug Formulation and Administration

Fibrin Sealant is a two-component biological adhesive comprising a concentrated preparation of human fibrinogen and a concentrated preparation of human thrombin prepared from screened, pooled human plasma by Cohn fractionation with cold alcohol. Each kit contains 50-90 mg of high concentrated fibrinogen in 1 ml of water for injection and 500 IU of thrombin in 1 ml calcium chloride.

The individual components were prepared and withdrawn into syringes to which the provided mixing device was attached. Depressing the syringe plungers forced the component solutions into a mixing spray to form a thin layer.

5. End-Points Measurement

Two weeks after model establishment, the modified peritoneal cancer index (PCI), which is based upon the number and the size of the cancer nodules formed on the peritoneum and mesentery surface, was scored according to the following criteria.

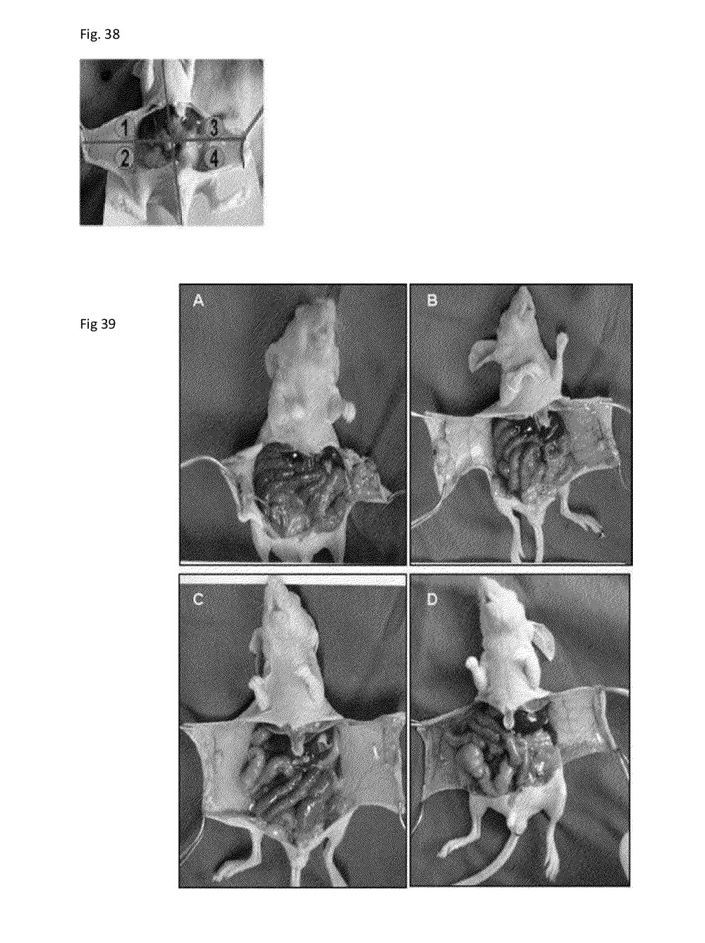

As shown in FIG. 38, the peritoneal surface of the abdomen and pelvis were divided into 4 identified regions. For each region, a Lesion Size (LS) score was calculated for the largest tumor in that region (not the number of tumors in the area, just the size of the largest tumor in that particular region).
If there were no tumor nodules in a region, a score of zero was given to that region (LS-0).
If tumor nodules in a region were smaller than 2 mm, an LS score of one (LS-1) was given to that region.
If a region had tumor nodules from 4 mm present, it was given a lesion size score of two (LS-2).
If a region had tumor nodules greater than 5 mm or if it had tumors that converged Gained together), that section was given a score of three (LS-3).

FIG. 38 shows four regions (upper-left, upper-right, lower-left, lower-right quarters) of peritoneal surfaces of abdomen

The lesion size scores for each of the 4 regions were added together. The highest score possible was 12 (4 times 3).

6. Mice Sacrifice and Sample Collection

All the mice were sacrificed using anesthesia overload. Peritoneal dissemination was assessed by the number of nodules larger than 1 mm in diameter and by the tumor volume score. Tumors were surgically removed and split in half at the indicated time points. One-half of the tumor was immediately snap-frozen in liquid nitrogen and stored at −80° C. until analysis. One-half of the tumor was fixed by 10% neutral buffered formalin (Richard-Allan Scientific, Kalamazoo, Mich.) for 24 hrs and embedded by paraffin.

Results

(1) Gross Appearance in the Peritoneal Surface:

Two weeks after tumor cell implantation, peritoneal dissemination was identified macroscopically as small nodules growth on the surface of the small intestine and peritoneum, as well as the mesentery. A significantly larger number of peritoneal dissemination nodules were observed in the normal saline treatment group than those in the other treatment groups (FIG. 39).

(2) Scores of Peritoneal Cancer Index (PCI) in FS Treatment Group and NS Group

The mean tumor volume score of PCI in control group was significantly higher than that in the other groups, with significant difference between groups (P<0.05).

Conclusions

Herein, the results of both in vitro and in vivo studies demonstrated the efficacy and potential synergistic efficacy of FS combined with RAAS 1 to 45 FU, as an adjuvant strategy in preventing gastric cancer dissemination with the mechanism of creating a compact mechanical barrier and cytotoxic efficacy.

### FIG. 39 Intraperitoneal Tumor Growth in FS Treatment Group, Sino-Fuan Group, Combination Group and Control Groups in Gastric Cancer Models

A: in the control group, large and fused tumor growth was observed in NS treatment groups.
B: in FS treatment group, small, isolated and well-capsulized tumor nodules were found
C: in the Sino-fuan group, smaller nodules were found, and even some un-degraded agents were still left
D: in the combination group, tiny tumor nodules were found, synergistic anti-tumor efficacy of FS plus Sino-Fuan, a slow-released chemotherapeutic agent, was demonstrated in this group

### Conclusion:

A kit of lyophilized or frozen high concentrate fibrinogen in which 5% A1AT will be added into the final bulk and or 5% A1AT as a diluent for high concentrate fibrinogen and new found proteins KH30 (SEQ ID NO: 1), KH31 (SEQ ID NO: 2), KH32 (SEQ ID NO: 3), KH44 (SEQ ID NO: 5), KH46 (SEQ ID NO: 7), KH47 (SEQ ID NO: 8), and KH 52 (SEQ ID NO: 4) in which the KH good healthy cells are present have inhibited the growth of tumor in gastric cancer, lung cancer, colon cancer and breast cancer and eventually will inhibit all other solid cancer tumor growth.
The combination of lyophilized or frozen high concentrate fibrinogen with the lyophilized or frozen thrombin used to compound the glue membrane, the diameter of which is less than 0.005 micrometers may help to prevent the spread of the cancer cells whose micrometer is larger than 0.005 micrometer into the good healthy cells.
The heating up preferably to 101° C. will kill both enveloped and non enveloped virus in high concentrate FNG, together with the addition of 5% Alpha 1 Antitrypsin increase the stability and durability of the fibrin sealant membrane in mice up to 52 days.

### RAAS

Title: Anti-tumor efficacy of high concentrated fibrinogen enriched a1at thrombin and AFOD in patient-derived tumor xenograft (PDX) models in nude mice.

### Description:

Patient-derived colorectal tumor xenograft (PDX) model was used to evaluate the anti-cancer efficacy of the high concentrated fibrinogen enriched a1at thrombin and AFOD at different 3 doses. The results showed that high concentrated fibrinogen enriched a1at thrombin and AFOD at all doses significantly inhibited the growth of PDX tumors implanted at 4 different locations of the peritoneum while having minor effects on mice body weights, which indicated high concentrated fibrinogen enriched a1at thrombin and AFOD is a potent anti-cancer agent on colorectal cancer with a limited side effect.

Subject: high concentrated fibrinogen enriched a1 at thrombin and AFOD, fibrinogen, thrombin, patient-derived tumor xenograft model, colorectal cancer

### Quotation: RAAS-20110926

### Summary

Patient-derived colorectal tumor xenograft (PDX) models (CO-04-0001 or CO-04-0002) were used to evaluate the anti-tumor efficacy of high concentrated fibrinogen enriched A1At thrombin and AFOD at 3 doses. PDX tumors (CO-04-0001 or CO-04-0002) were implanted at 4 different locations in peritoneal cavity, and high concentrated fibrinogen enriched A1At thrombin and AFOD, or a control agent was applied to peritoneum before and after tumor implantation. 30 days after implantation, the mice were sacrificed and tumors were dissected and weighed. The final tumor weights for all groups were statistically analyzed by one-way ANOVA with the significance level set at 0.05.

The data show that high concentrated fibrinogen enriched a1at thrombin and AFOD at all 3 doses exhibits significant inhibitory effects on tumor growth in PDX colorectal cancer model while no significant toxicity was observed, which indicates high concentrated fibrinogen enriched A1At thrombin and AFOD is a potential anti-tumor agent in colorectal cancer, warranting further development of the agent for clinical application.

Note: The page numbers presented in this table of contents are not consistent with the page numbering in this specification.

TABLE OF CONTENTS

| 1. | DETAILS OF FACILITY, PERSONNEL AND DATA LOCATION | | 4 |
|---|---|---|---|
| 2. | INTRODUCTION | | 4 |
| 3. | METHODS | | 5 |
| | 3.1. | Experimental Preparations | 5 |

| | | |
|---|---|---|
| 3.1.1. | Animal preparation | 5 |
| 3.1.2. | Tumor tissue preparation | 5 |
| 3.1.3. | Formulation | 5 |

| | | |
|---|---|---|
| 3.2. | Experimental Protocol | 5 |

| | | |
|---|---|---|
| 3.2.1. | Establishment of Xenograft Model and Treatment | 5 |
| 3.2.2. | Evaluation of the Anti-Tumor Activity | 8 |

| | | |
|---|---|---|
| 3.3. | Drugs and Materials | 8 |
| 3.4. | Data Analysis | 8 |

| | | |
|---|---|---|
| 3.4.1. | Relative Chage of Body Weight (RCBW) | 8 |
| 3.4.2. | Tumor weight | 8 |
| 3.4.3. | Statistical analysis | 8 |

| 4. | RESULTS | | 8 |
|---|---|---|---|
| | 4.1. | Inhibition on tumor growth | 8 |
| | 4.2. | Effect on Body weight | 9 |

| | | |
|---|---|---|
| 5. | DISCUSSION | 9 |
| 6. | REFERENCES | 11 |
| 7. | FIGURES | 12 |
| | FIG. 26.18. Anti-tumor efficacy of test agent in PDX model CO-04-0002. | 12 |
| | FIG. 26.22. Anti-tumor efficacy of test agent in PDX model CO-04-0002 and | 13 |

CO-04-0001

| | |
|---|---|
| FIG. 26.23. Photographs of tumors dissected from abdominal cavity of each | 14 |

group

| | | |
|---|---|---|
| | FIG. 26.24. Relative change of body weight (%) of different groups | 15 |
| 8. | TABLES | 16 |
| | Table 8.2. Ratios of palpable tumors observed in each group | 16 |
| | Table 8.3. Relative change of body weight (%) of different groups | 17 |

1. Details of Facility, Personnel and Data Location

| | |
|---|---|
| Sponsor: | RAAS |
| Test Facility: | WuXi AppTec Animal facility in 90 Delin Road, |
| | Waigaoqiao Free Trade Zone, Shanghai 200131, |
| | P.R.China. |
| Date of Work: | Commenced: Oct. 17, 2011 |
| | Completed: Nov. 25, 2011 |
| Personnel Involved: | Yunbiao Yan scientist BS |
| | Guizhu Yang scientist BS |
| Study Director/ | Douglas Fang Senior director Ph.D |
| Senior Scientist: | |

### Location of Raw Data, Original Protocols, Experimental Details and Report

The studies described in this report were carried out on behalf of RAAS at external laboratories:

All raw data, protocols and experimental details pertaining to these studies and the original of the report will be held in the Archive of WuXi AppTec in 90 Delin Road, Waigaoqiao Free Trade Zone, Shanghai 200131, P.R.China.

### 2. Introduction

The aim of the study was to test anti-tumor efficacy of high concentrated fibrinogen enriched A1At thrombin and AFOD in patient-derived colorectal tumor xenograft (PDX) model in nude mice.

The model used in the study was derived from surgically resected, fresh patient tumor tissues. The first generation of the xenograft tumors in mice was termed passage 0 (P0), and so on during continual implantation in mice. The passage of xenograft tumors at P2 (CO-04-0002) or P3 (CO-04-0001) were used in this study.

All the experiments were conducted in the AAALAC-accrediated animal facility in compliance with the protocol approved by the Institutional Animal Care and Use Committee (IACUC).

### 3. Methods

### 3.1. Experimental Preparations

### 3.1.1. Animal Preparation

Female Balb/c nude mice, with a body weight of approximately 20 grams, were obtained from an approved vendor (Sino-British SIPPR/BK Lab. Animal Co. Ltd., Shanghai, China).

### Acclimation/Quarantine:

Upon arrival, animals were assessed as to their general health by a member of a veterinary staff or authorized personnel. Animals were acclimated for at least 3 days (upon arrival at the experiment room) before being used for the study.

### Animal Husbandry:

Animals were housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Sino-British SIPPR/BK Lab. Animal Co. Ltd., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcome of the study.

### 3.1.2. Tumor Tissue Preparation

The colorectal xenograft tumor models were established from surgically resected clinical tumor samples. The first generation of the xenograft tumors in mice is termed passage 0 (P0), and so on during continual implantation in mice. The tumor tissues at passage 2 (CO-04-0002) or P3 (CO-04-0001) were used in this study.

### 3.1.3. Formulation

Test agent: high concentrated fibrinogen enriched a1at thrombin and AFOD were provided by RAAS and prepared by RAAS scientist during experiment before use.

Control agent: Matrigel (BD Biosciences; cat. #356234).

### 3.2. Experimental Protocol

### 3.2.1. Establishment of Xenograft Model and Treatment

### Grouping and Treatment

Nude mice were assigned to 6 different groups with 12-17 mice/group and each group received different treatment as shown in Table 9.1.

8 out 17 (9 left) mice in high dose high concentrated fibrinogen enriched a1at thrombin and AFOD group died during the first experiment using PDX model CO-04-0002. To make up for the loss of mice in high dose group, 6 additional mice were implanted with tumor fragments collected from model CO-04-0001 and treated with high dose high concentrated fibrinogen enriched a1at thrombin and AFOD. So the total mice number in high dose group was 15.

TABLE 9.1
Grouping and the treatment.

| Group | Treatment | N | Remarks |
|---|---|---|---|
| 1 | Sham-operation | 12 | Open up the abdominal cavity and |
| | | | close it with sutures. (No implants) |
| 2 | Vehicle control | 12 | Implant tumor fragments of 20 mm³ in |
| | | | size into 4 corners of abdominal cavity. |
| 3 | Matrigel | 12 | Embed tumor fragments of 20 mm³ in |
| | | | Matrigel. Implant the tumor fragments |
| | | | into 4 corners of abdominal cavity. Close |
| 4 | 3 ml of high concentrated | 9 + 6 | Spray high concentrated fibrinogen |
| | fibrinogen enriched a1at | | enriched a1at thrombin and Afod to cover |
| | thrombin and Afod (high | | the entire peritoneum and the internal |
| | dose) on the peritoneum | | organs. Implant the tumor fragments of |
| | in abdominal cavity of [symbol] | | 20 mm³ into 4 corners of abdominal |
| 5 | 2 ml of high concentrated | 12 | Spray high concentrated fibrinogen |
| | fibrinogen enriched a1at | | enriched a1at thrombin and Afod to cover |
| | thrombin and Afod | | the entire peritoneum and the internal |
| | (moderate dose) on the | | organs. Implant the tumor fragments of 20 |
| | peritoneum in abdominal [symbol] | | mm³ into 4 corners of abdominal cavity. |
| 6 | 1 ml of high concentrated | 13 | Spray high concentrated fibrinogen |
| | fibrinogen enriched a1at | | enriched a1at thrombin and Afod to cover |
| | thrombin and Afod (low | | the entire peritoneum and the internal |
| | dose) on the peritoneum | | organs. Implant the tumor fragments of |
| | in abdominal cavity of [symbol] | | 20 mm³ into 4 corners of abdominal |
| Total | | 76 | |

[symbol]

indicates data missing or illegible when filed

### Experiment Procedures

A. The animal was anesthetized by i.p. injection of sodium pentobarbital at 60-70 mg/kg. Disinfect the abdominal skin of nude mice with 70% ethanol solution. Open up the abdominal wall along the midline of the ventral surface to expose the peritoneal surface.
B. The surgeries for different groups were done according to table 9.1.
C. For groups using test agent, high concentrated fibrinogen enriched a1at thrombin and Afod was then applied on the peritoneal surface.
D. Tumor fragments were implanted at 4 different locations of the peritoneal cavity. The test agent acted as a glue to hold the fragments.
E. The test agent was applied again on the surface of tumor fragments and peritoneum.
F. After the fibrin membrane formed completely, the peritoneal cavity was closed. G. In Matrigel control groups, tumor fragments were embedded into matrigel before implantation.
H. Postoperative cares followed protocol SOP-BEO-0016-1.0.
I. Mice were palpated for tumors 2 weeks after implantation. The ratio of palpable tumors observed in each group was recorded.
J. 30 days after implantation, the mice were sacrificed and tumors were dissected and weighed.
K. The tissues surrounding tumor fragments were also checked to find out whether the tumors had spread to other organ sites within the peritoneal cavity.
L. Pictures of tumor-bearing mice and dissected tumors were taken.
M. If possible, tumor sizes were measured twice per week. Tumor volumes (mm³) are obtained by using the following formula: volume=(W2×L)/2 (W, width; L, length in mm of the tumor).
N. During the experiment, health conditions of mice were observed daily. Body weights of mice were monitored twice per week.

### 3.2.2. Evaluation of the Anti-Tumor Activity

Health conditions of mice were observed daily. Body weights were measured twice per week during the treatment. Mice were palpated for tumors 2 weeks after implantation. The ratio of palpable tumors observed in each group was recorded. 30 days after treatment, all mice were euthanized with CO₂ and cervical dislocation was followed after respiratory arrest. Routine necropsy was performed to detect any abnormal signs of each internal organ with specific attention to metastases. Each tumor was removed and weighted.

### 3.3. Drugs and Materials

High concentrated fibrinogen enriched a1at thrombin and Afod were provided by RAAS; Matrigel was from BD Biosciences (San Jose, Calif., cat. #356234). Digital caliper was from Sylvac, Switzerland.

### 3.4. Data Analysis

### 3.4.1. Relative Change of Body Weight (RCBW)

Relative change of body weight (RCBW) was calculated based on the following formula: RCBW (%)=(BWi−BW0)/BW0×100%; BWi was the body weight on the day of weighing and BW0 was the body weight before surgery.

### 3.4.2. Tumor Weight

Tumors from each mouse were pooled and weighed after sacrificing mice.

### 3.4.3. Statistical Analysis

Data were expressed as mean±SEM; the difference between the groups was analyzed for significance using one-way ANOVA and Dunnett's test.

### 4. Results

### 4.1. Tumor Growth Inhibition

Three weeks after implantation, all 12 mice in vehicle control group showed palpable tumors, while only less than 2 palpable tumors were found in each test agent-treated group. High concentrated fibrinogen enriched a1at thrombin and Afod treatment delayed the appearance of palpable tumors as shown in table 9.2, indicating high concentrated fibrinogen enriched a1at thrombin and Afod inhibited the growth of implanted colorectal tumors in vivo.

Thirty days after implantation, tumors in vehicle control group and matrigel group reached more than 1 g on average. Conversely, tumor weights in test agent high, moderate and low dose groups were 0.49 g (0.35 if when two models are combined), 0.28 g and 0.13 g, respectively. Compared with the vehicle control, high concentrated fibrinogen enriched a1at thrombin and Afod demonstrated significant anti-tumor activities in colorectal cancer PDX model at all 3 doses. The inhibition on tumor growth were shown in FIGS. 26.18 & 26.22 and table 9.2.

### 4.2. Effect on Body Weight

Loss of body weight, a sign of toxicity, was not seen in test agent-treated groups, which only showed minor decrease in weight gain. Mortalities were observed within 3 days after surgery and treatment in high dose of test agent group, which may due to the large volume (3 ml) of test agent used in this group.

The effect on body weight was shown in FIG. 26.24 and table 9.3.

### 5. Discussion

Patient-derived colorectal tumor xenograft (PDX) model was used to evaluate the anti-cancer efficacy of the high concentrated fibrinogen enriched a1at thrombin and Afod at 3 doses. PDX tumors (CO-04-0001 and CO-04-0002) were implanted at 4 different locations in peritoneal cavity, and high concentrated fibrinogen enriched a1at thrombin and Afod, or a control agent was applied to peritoneum before and after tumor implantation.

Mice were palpated for tumors 2 weeks after implantation. The ratio of palpable tumors observed in each group was recorded. Test agent treatment inhibited the tumor growth as shown by the delayed appearance of palpable tumors. There weeks after implantation, all 12 mice in vehicle control group showed palpable tumors, while only less than 2 palpable tumors were found in each test agent-treated group (Table 9.2).

Thirty days after implantation, the mice were sacrificed and tumors were dissected and weighed. Tumors in vehicle control group and matrigel group reached more than 1 g on average. Conversely, tumor weights in test agent high, moderate and low dose groups were 0.49 g (0.35 when two models are combined), 0.28 g and 0.13 g, respectively. Compared with the vehicle control, high concentrated fibrinogen enriched a1at thrombin and Afod demonstrated significant anti-tumor activities in colorectal cancer PDX model at all 3 doses. Matrigel has been commonly used to facilitate the establishment of human tumor xenografts in rodents. In this study, matrigel group promoted an increase in tumor weight thought the increase was not statistically significant.

Loss of body weight, a sign of toxicity, was not seen in all test agent-treated groups, in which the animals only showed a minor decrease in weight gain compared to sham-operated group. Mortalities observed in test agent high dose group right after the surgery could be due to large volume of test agent (3 ml) used in this group. The mice of vehicle and matrigel groups started to loss body weights 2 weeks after surgery due to the continuously increased tumor volumes.

In summary, the results show that high concentrated fibrinogen enriched a1at thrombin and Afod at all doses significantly inhibits the growth of colorectal tumors in vivo while having minor effects on mice body weight. The results suggest that high concentrated fibrinogen enriched a1at thrombin and Afod is a potent anti-tumor agent in colorectal cancer.

### 6. References

N/A

### 7. Figures

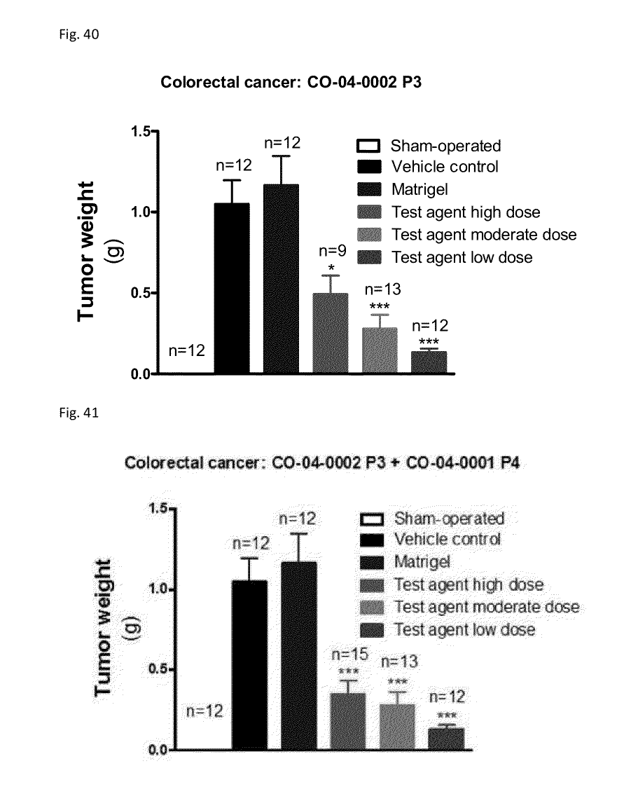

FIG. 40. Anti-Tumor Efficacy of High Concentrated Fibrinogen Enriched a1at Thrombin and Afod in PDX Model CO-04-0002.

Tumor weights from model CO-04-0002 were used. Data are expressed as mean±SEM. *<0.05, *“<0.001 vs vehicle group (one-way ANOVA and Dunnett's test).

### FIG. 41. Anti-Tumor Efficacy of High Concentrated Fibrinogen Enriched Alat Thrombin and Afod in PDX Model CO-04-0002 and CO-04-0001.

Tumor weights of 6 mice from model CO-04-0001 were combined with the data from model CO-04-0002. There were 15 mice in total in high dose of test agent group. Data are expressed as mean±SEM. *<0.05, *”<0.001 vs vehicle group (one-way ANOVA and Dunnett's test).

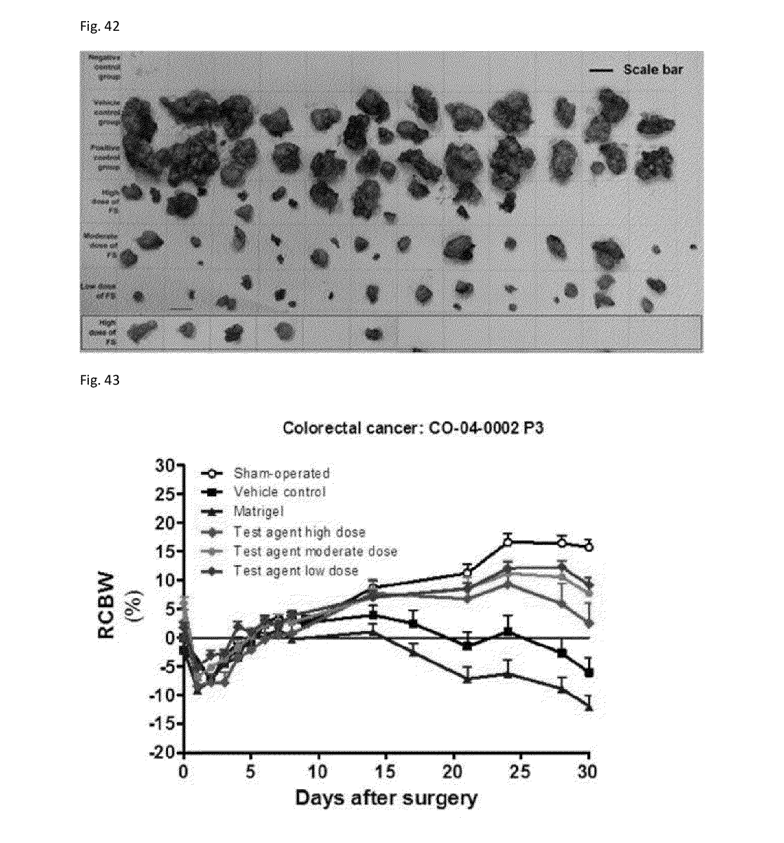

FIG. 42. Photographs of Tumors Dissected from Abdominal Cavity of Each Group.

Tumors from each mouse were pooled and weighed. The tumors in frame were from model CO-04-0002 (upper panels) and the rest were form model CO-04-0001 (bottom panel). Scale bar, 1 cm.

### FIG. 43. Relative Change of Body Weight (%) of Different Groups.

Data are expressed as mean±SEM. Relative change of body weight (RCBW) was calculated based on the following formula: RCBW (%)=(BWi−BW0)/BW0×100%; BWi was the body weight on the day of weighing and BW0 was the body weight before surgery.

### Confidential

### 8. Tables

TABLE 9.2
Ratios of palpable tumors observed in each group.

Days
after

[symbol]

| | | | | | | | | |
|---|---|---|---|---|---|---|---|---|
| Sham- | 0/12 | 0/12 | 0/12 | 0/12 | 0/12 | 0/12 | 0/12 | 0/12 |
| oper- | | | | | | | | |
| ated | | | | | | | | |
| [symbol] | | | | | | | | |
| vehicle | 0/12 | 1/12 | 4/12 | 4/12 | 8/12 | 12/12 | 12/12 | 12/12 |
| control | | | | | | | | |
| [symbol] | | | | | | | | |
| | 1/12 | 3/12 | 5/12 | 5/12 | 5/12 | 8/12 | 11/12 | 12/12 |
| high | [symbol] | [symbol] | [symbol] | [symbol] | [symbol] | [symbol] | [symbol] | [symbol] |
| dose of | | | | | | | | |
| [symbol] | | | | | | | | |
| mod- | 0/13 | 0/13 | 1/13 | 1/13 | 1/13 | 2/13 | 2/13 | 5/13 |
| erate | | | | | | | | |
| dose of | | | | | | | | |
| test | | | | | | | | |
| agent | | | | | | | | |
| low | [symbol] | [symbol] | [symbol] | [symbol] | [symbol] | [symbol] | [symbol] | [symbol] |
| dose of | | | | | | | | |
| [symbol] | | | | | | | | |

[symbol]

indicates data missing or illegible when filed

[symbol]

Mice were palpated for tumors at 15, 16, 17, 18, 20, 21, 24, 28 days after implantation. The ratios of palpable tumors observed in each group were recorded.

### Confidential

TABLE 9.3
Relative change of body weight (%) of different groups.

| | | Days after surgery | | | | | | | |
|---|---|---|---|---|---|---|---|---|---|
| | | 0 → | 1 → | 2→ | 3 → | 4 → | 5→ | 6 → | 7 → |
| | | RCBW | RCBW | RCBW | RCBW | RCBW | RCBW | RCBW | RCBW |
| Group → | | (%) | (%) | (%) | (%) | (%) | (%) | (%) | (%) |
| Sham- | Mean | 0.00 | → | −7.41 | −3.46 | −0.73 | −1.24 | 3.08 | 3.18 |
| operated | SD→ | 0.00 | → | 2.98 | 3.03 | 3.03 | 4.19 | 2.94 | 3.18 |
| | SEM | 0.00 | → | 0.86 | 0.87 | 0.88 | 1.21 | 0.85 | 0.92 |
| Vehicle | Mean | −2.14 | → | −7.06 | −4.16 | −2.12 | −0.99 | 2.37 | 2.24 |
| control | SD→ | 0.87 | → | 2.62 | 2.93 | 3.82 | 3.99 | 4.06 | 4.10 |
| | SEM | 0.25 | → | 0.76 | 0.85 | 1.10 | 1.15 | 1.17 | 1.18 |
| Matrigel | Mean | −1.97 | −9.20 | −7.41 | −4.43 | −3.47 | 0.62 | 0.09 | 1.50 |
| | SD→ | 1.14 | 2.37 | 3.60→ | 2.54 | 2.17 | 2.74 | 2.46 | 3.07 |
| | SEM | 3.33 | 0.68 | 1.04 | 0.73 | 0.63 | 0.79 | 0.71 | 0.89 |
| High | Mean | 2.66 | −8.41 | −7.80 | −7.78 | −3.48 | −2.09 | −0.26 | 0.50 |
| dose Of | SD→ | 6.60 | 2.76 | 4.29 | 5.40 | 3.19 | 5.68 | 6.05 | 6.52 |
| test | SEM | 2.20 | 0.92 | 1.43 | 1.80 | 1.06 | 1.89 | 2.02 | 2.17 |
| agent | | | | | | | | | |
| Moderate | Mean | 5.95 | −6.73 | −5.23 | −3.70 | −1.70 | 0.30 | 2.37 | 2.55 |
| dose of | SD→ | 4.04 | 2.13 | 2.43 | 4.38 | 4.61 | 5.08 | 4.15 | 5.29 |
| test | SEM | 1.12 | 0.59 | 0.67 | 1.22 | 1.28 | 1.41 | 1.15 | 1.47 |
| agent | | | | | | | | | |
| Low | Mean | 1.82 | −5.27 | −3.04 | −2.75 | 1.93 | 1.00 | 2.86 | 2.11 |
| Dose of | SD→ | 2.74 | 2.24 | 2.55 | 2.53 | 2.90 | 2.15 | 3.00 | 2.89 |
| test | SEM | 0.79 | 0.65 | 0.65 | 0.73 | 0.84 | 0.62 | 0.87 | 0.84 |
| agent | | | | | | | | | |

| | | Days after surgery | | | | | | |
|---|---|---|---|---|---|---|---|---|
| | | 8→ | 9 → | 14 → | 21 → | 24 → | | 28 |
| | | RCBW | RCBW | RCBW | RCBW | RCBW | RCBW | RCBW |
| Group → | | (%) | (%) | (%) | (%) | (%) | (%) | (%) |
| Sham- | Mean | 2.84 | 2.45 | 8.67 | 11.20 | 16.61 | 16.46 | 15.70 |
| operated | SD→ | 3.18 | 4.21 | 4.21 | 5.70 | 5.31 | 4.87 | 5.07 |
| | SEM | 0.92 | 1.21 | 1.21 | 1.65 | 1.53 | 1.41 | 1.46 |
| Vehicle | Mean | 3.38 | 2.55 | 3.92 | −1.48 | 1.02 | −2.70 | −6.01 |
| control | SD→ | 3.85 | 4.34 | 5.74 | 8.75 | 9.95 | 9.36 | 8.72 |
| | SEM | 1.11 | 1.25 | 1.66 | 2.53 | 2.87 | 2.70 | 2.52 |
| Matrigel | Mean | −0.29 | → | 1.00 | −7.17 | −6.25 | −8.92 | — |
| | SD→ | 3.35 | → | 4.95 | 7.26 | 8.33 | 7.24 | 6.50 |
| | SEM | 0.97 | → | 1.43 | 2.10 | 2.40 | 2.09 | 1.88 |
| High | Mean | 0.73 | → | 7.71 | 6.72 | 9.28 | 5.90 | 2.48 |
| dose Of | SD→ | 5.77 | → | 7.22 | 7.93 | 8.90 | 10.53 | 10.39 |
| test | SEM | 1.92 | → | 2.41 | 2.64 | 2.97 | 3.51 | 3.46 |
| agent | | | | | | | | |
| Moderate | Mean | 2.66 | → | 7.00 | 8.46 | 11.16 | 10.55 | 7.68 |
| dose of | SD→ | 5.85 | → | 5.58 | 7.03 | 7.98 | 10.25 | 9.57 |
| test | SEM | 1.62 | → | 1.55 | 1.95 | 2.21 | 2.84 | 2.66 |
| agent | | | | | | | | |
| Low | Mean | 3.85 | → | 7.08 | 7.08 | 12.03 | 12.27 | 9.18 |
| Dose of | SD→ | 3.17 | → | 3.78 | 3.78 | 3.65 | 3.77 | 4.16 |
| test | SEM | 0.92 | → | 1.09 | 1.09 | 1.05 | 1.09 | 1.20 |
| agent | | | | | | | | |

Relative change of body weight (RCBW) was calculated based on the following formula: RCBW (%)=(BWi−BW0)/BW0×100%; BWi was the body weight on the day of weighing and BW0 was the body weight before surgery.

### RAAS

a.
Title: Anti-tumor efficacy of high concentrated fibrinogen enriched a1at thrombin and Afod in a patient-derived tumor xenograft (PDX) model of lung cancer in nude mice.
Description: Patient-derived tumor xenograft (PDX) model of lung cancer was used to evaluate the anti-cancer efficacy of high concentrated fibrinogen enriched a1at thrombin and Afod at different 3 doses. The results showed that high concentrated fibrinogen enriched a1at thrombin and afod at all doses significantly inhibited the growth of PDX tumors implanted at 4 different locations of the peritoneum while having minor effects on mice body weights, which indicates high concentrated fibrinogen enriched a1at thrombin and Afod is a potent anti-cancer agent on lung cancer with a limited side effect.
Subject: high concentrated fibrinogen enriched a1at thrombin and Afod, patient-derived tumor xenograft model, lung cancer

### Quotation: RAAS-20111029

General Notes for Writing this Module:

Do not change the font or font size, styles within this document or the margins, as all of these are dictated by @standard compliance guidelines and/or publishing requirements.

Blue text represents generic text. Select the blue text and replace it with the information appropriate to your report. Any blue text will be changed to black during publishing.

If you must paste text from another module or document, do not do a “regular” paste, as you may bring non-compliant formatting with the text. Instead, choose Paste Special from the Edit menu, select Unformatted text from the dialog box, and click OK. You can then apply the correct style using only the @standard styles from the Style drop-down in the upper-left corner of the Word screen.

Please read all INFO comments and hidden text within this document!

### Summary

Patient-derived tumor xenograft (PDX) model of lung cancer (LU-01-0032) was used to evaluate the anti-tumor efficacy of high concentrated fibrinogen enriched a1at thrombin and Afod at 3 doses. PDX tumors (LU-01-0032) were implanted at 4 different locations in peritoneal cavity, and high concentrated fibrinogen enriched a1at thrombin and Afod or a control agent was applied to peritoneum before and after tumor implantation. Forty five days after implantation, the mice were sacrificed and tumors were removed and weighed. The final tumor weights for all groups were statistically analyzed by one-way ANOVA with the significance level set at 0.05.

The data show that high concentrated fibrinogen enriched a1at thrombin and Afod at all 3 doses exhibits significant inhibitory effects on tumor growth in the lung cancer model while no significant toxicity was observed, which indicates high concentrated fibrinogen enriched a1at thrombin and Afod was a potential anti-tumor agent in lung cancer, warranting further development of high concentrated fibrinogen enriched a1at thrombin and Afod for clinical application.

TABLE OF CONTENTS
Research_WW Research Report Template
Lisa Ross (Template Owner): contact for template content changes.
WW-Template Team: contact for template technical issues.

| 1. DETAILS OF FACILITY, PERSONNEL AND DATA LOCATION | | | 58 |
|---|---|---|---|
| 2. Introduction | | | 58 |
| 3. METHODS | | | 59 |
| | 3.1. | Experimental Preparations | 59 |

| | | |
|---|---|---|
| 3.1.1. | Animal preparation | 59 |
| 3.1.2. | Tumor tissue preparation | 59 |
| 3.1.3. | Formulation | 60 |

| | | |
|---|---|---|
| 3.2. | Experimental Protocol | 60 |

| | | |
|---|---|---|
| 3.2.1. | Establishment of Xenograft Model and Treatment | 60 |
| 3.2.2. | Evaluation of the Anti-Tumor Activity | 62 |

| | | |
|---|---|---|
| 3.3. | Drugs and Materials | 62 |
| 3.4. | Data Analysis | 63 |

| | | |
|---|---|---|
| 3.4.1. | Relative Chage of Body Weight (RCBW) | 63 |
| 3.4.2. | Tumor weight | 63 |
| 3.4.3. | Statistical analysis | 63 |

| 4. RESULTS | | | 63 |
|---|---|---|---|
| | 4.1. | Tumor growth inhibition | 63 |
| | 4.2. | Effect on Body weight | 64 |

| 5. DISCUSSION | | 64 |
|---|---|---|
| 6. REFERENCES | | 66 |
| 7. FIGURES | | 67 |
| | FIG. 1. Anti-tumor efficacy of high concentrated fibrinogen enriched a1at thrombin and | 67 |
| | Afod in PDX model LU-01-0032 | |
| | FIG. 2. Photographs of tumors dissected from abdominal cavity of each group | 67 |
| | FIG. 3. Ratios of mice with palpable tumors observed in each group | 67 |
| | FIG. 4. Relative change of body weight (%) of different groups | 67 |
| 8. TABLES | | 68 |
| | Table 2. Ratios of palpable tumors observed in each group | 68 |
| | Table 3. Relative change of body weight (%) of different groups | 70 |

### 1. Details of Facility, Personnel and Data Location

| | |
|---|---|
| Sponsor: | RAAS |
| Test Facility: | WuXi AppTec Animal facility in 90 Delin Road, |
| | Waigaoqiao Free Trade Zone, Shanghai 200131, |
| | P.R.China. |
| Date of Work: | Commenced: Oct. 17, 2011 |
| | Completed: Nov. 25, 2011 |
| Personnel Involved: | Yunbiao Yan scientist BS |
| | Guizhu Yang scientist BS |
| Study Director/ | Douglas Fang Senior director Ph.D |
| Senior Scientist: | |

### Location of Raw Data, Original Protocols, Experimental Details and Report

### Either:

### Or:

The studies described in this report were carried out on behalf of RAAS at external laboratories:

All raw data, protocols and experimental details pertaining to these studies and the original of the report will be held in the Archive of WuXi AppTec in 90 Delin Road, Waigaogiao Free Trade Zone, Shanghai 200131, P.R.China.

### 2. Introduction

The aim of the study was to test anti-tumor efficacy of high concentrated fibrinogen enriched a1at thrombin and Afod in patient-derived lung tumor xenograft (PDX) model in nude mice.

The model used in the study was derived from surgically resected, fresh patient tumor tissues. The first generation of the xenograft tumors in mice was termed passage 0 (P0), and so on during continual implantation in mice. The passage of xenograft tumors at P5 (LU-01-0032) were used in this study.

All the experiments were conducted in the AAALAC-accrediated animal facility in compliance with the protocol approved by the Institutional Animal Care and Use Committee (IACUC).

### 3. Methods

### 3.1. Mental Preparations

### 3.1.1. Animal Preparation

Female Balb/c nude mice, with a body weight of approximately 20 grams, were obtained from an approved vendor (Sino-British SIPPR/BK Lab. Animal Co. Ltd., Shanghai, China).

### Acclimation/Quarantine:

Upon arrival, animals were assessed as to their general health by a member of a veterinary staff or authorized personnel. Animals were acclimated for at least 3 days (upon arrival at the experiment room) before being used for the study.

### Animal Husbandry:

Animals were housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Sino-British SIPPR/BK Lab. Animal Co. Ltd., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcome of the study.

### 3.1.2. Tumor Tissue Preparation

The lung xenograft tumor models were established from surgically resected clinical tumor samples. The first generation of the xenograft tumors in mice is termed passage 0 (P0), and so on during continual implantation in mice. The tumor tissues at passage 5 (LU-01-0032) were used in this study.

### 3.1.3. Formulation

High concentrated fibrinogen enriched a1at thrombin and Afod were provide by RAAS and prepared by RAAS scientist during experiment before use.

Matrigel (BD Biosciences; cat. #356234).

### 3.2. Experimental Protocol

### 3.2.1. Establishment of Xenograft Model and Treatment

### Grouping and Treatment

Nude mice were assigned to 6 different groups with 11-19 mice/group and each group received different treatments as shown in Table 1.

TABLE 1
Grouping and the treatment.

| Group | Treatment | N | Remarks |
|---|---|---|---|
| 1 | Sham-operation | 12 | a. Open up the abdominal cavity and |
| | | | close it with sutures. (No implants) |
| 2 | Vehicle control | 13 | b. Implant tumor fragments of 20 mm³ |
| | | | in size into 4 corners of abdominal cavity. |
| | | | Close body with sutures. |
| 3 | Matrigel | 13 | c. Embed tumor fragments of 20 mm³ |
| | | | in Matrigel. Implant the tumor fragments into |
| | | | 4 corners of abdominal cavity. Close body |
| | | | with sutures. |
| 4 | 3 ml high concentrated | 19 | d. Spray high concentrated fibrinogen |
| | fibrinogen enriched a1at | | enriched a1at thrombin and Afod to cover |
| | thrombin and Afod (high | | the entire peritoneum and the internal |
| | dose) on the peritoneum in | | organs. Implant the tumor fragments of 20 |
| | abdominal cavity of nude | | mm³ into 4 corners of abdominal cavity. |
| | mice | | Close body with sutures. |
| 5 | 2 ml high concentrated | 14 | e. Spray high concentrated fibrinogen |
| | fibrinogen enriched a1at | | enriched a1at thrombin and Afod to cover |
| | thrombin and Afod | | the entire peritoneum and the internal |
| | (moderate dose) on the | | organs. Implant the tumor fragments of 20 |
| | peritoneum in abdominal | | mm³ into 4 corners of abdominal cavity. |
| | cavity of nude mice | | Close body with sutures. |
| 6 | 1 ml high concentrated | 11 | f. Spray high concentrated fibrinogen |
| | fibrinogen enriched a1at | | enriched a1at thrombin and Afod to cover |
| | thrombin and Afod (low | | the entire peritoneum and the internal |
| | dose) on the peritoneum in | | organs. Implant the tumor fragments of 20 |
| | abdominal cavity of nude | | mm³ into 4 corners of abdominal cavity. |
| | mice | | Close body with sutures. |
| Total | | 82 | |

### Experiment Procedures

A. Measured the body weight of each mouse before surgery.
B. The animal was anesthetized by i.p. injection of sodium pentobarbital at 60-70 mg/kg. Disinfect the abdominal skin of nude mice with 70% ethanol solution. Open up the abdominal wall along the midline of the ventral surface to expose the peritoneal surface.
C. The surgeries for different groups were done according to table 1.
D. For groups using test agent high concentrated fibrinogen enriched a1at thrombin and Afod, the test agent was then applied on the peritoneal surface.
E. Tumor fragments were implanted at 4 different locations of the peritoneal cavity. The test agent acted as a glue to hold the fragments.
F. The test agent high concentrated fibrinogen enriched a1at thrombin and Afod was applied again on the surface of tumor fragments and peritoneum.
G. After the fibrin membrane formed completely, the peritoneal cavity was closed.
H. In Matrigel control groups, tumor fragments were embedded into matrigel before implantation.
I. Postoperative cares followed protocol SOP-BEO-0016-1.0.
J. Mice were palpated for tumors 2 weeks after implantation. The ratio of palpable tumors observed in each group was recorded.
K. Forty five days after implantation, the mice were sacrificed and tumors were dissected and weighed.
L. The tissues surrounding tumor fragments were also checked to find out whether the tumors had spread to other organ sites within the peritoneal cavity.
M. Pictures of tumor-bearing mice and dissected tumors were taken.
N. If possible, tumor sizes were measured twice per week. Tumor volumes (mm³) are obtained by using the following formula: volume=(W2×L)/2 (W, width; L, length in mm of the tumor).
O. During the experiment, health conditions of mice were observed daily. Body weights of mice were monitored twice per week.

### 3.2.2. Evaluation of the Anti-Tumor Activity

Health conditions of mice were observed daily. Body weights were measured twice per week during the treatment. Mice were palpated for tumors 2 weeks after implantation. The ratio of palpable tumors observed in each group was recorded. 45 days after treatment, all mice were euthanized with CO₂ and cervical dislocation was followed after respiratory arrest. Routine necropsy was performed to detect any abnormal signs of each internal organ with specific attention to metastases. Each tumor was removed and weighted.

### 3.3. Drugs and Materials

High concentrated fibrinogen enriched a1at thrombin and Afod were provided by RAAS; Matrigel was from BD Biosciences (San Jose, Calif., cat. #356234).

Digital caliper was from Sylvac, Switzerland.

### 3.4. Data Analysis

### 3.4.1. Relative Change of Body Weight (RCBW)

Relative change of body weight (RCBW) was calculated based on the following formula: RCBW (%)=(BWi−BW0)/BW0×100%; BWi was the body weight on the day of weighing and BW0 was the body weight before surgery.

### 3.4.2. Tumor Weight

Tumors from each mouse were pooled and weighed after sacrificing mice.

### 3.4.3. Statistical Analysis

Data were expressed as mean±SEM; the difference between the groups was analyzed for significance using one-way ANOVA and Dunnett's test.

### 4. Results

### 4.1. Tumor Growth Inhibition

Four weeks after implantation, 9 out of 13 mice in vehicle control group showed palpable tumors, while only less than 5 palpable tumors were found in each high concentrated fibrinogen enriched a1at thrombin and Afod-treated group. High concentrated fibrinogen enriched a1at thrombin and Afod treatment delayed the appearance of palpable tumors as shown in table 2, indicating high concentrated fibrinogen enriched a1at thrombin and Afod inhibited the growth of implanted lung tumors in vivo. After sacrificing the mice, tumors were found in all the mice in vehicle control group, while some tumors completely regressed in several high concentrated fibrinogen enriched a1at thrombin and Afod-treated mice (FIG. 3).

Forty-five days after implantation, tumors in vehicle control group reached more than 0.7 g on average. Conversely, tumor weights in high concentrated fibrinogen enriched a1at thrombin and Afod high, moderate and low dose groups were 0.19 g, 0.16 g and 0.16 g, respectively. Compared with the vehicle control, high concentrated fibrinogen enriched a1at thrombin and Afod demonstrated significant anti-tumor activities in lung cancer PDX model at all 3 doses (FIG. 1-2).

The inhibition on tumor growth were shown in FIG. 1-3 and table 2.

### 4.2. Effect on Body Weight

Loss of body weight, a sign of toxicity, was not seen in high concentrated fibrinogen enriched a1at thrombin and Afod-treated groups, indicating the test agent has no/little side effects.

The effect on body weight was shown in FIG. 4 and table 3.

### 5. Discussion

Patient-derived tumor xenograft (PDX) model of lung cancer was used to evaluate the anti-cancer efficacy of the high concentrated fibrinogen enriched a1at thrombin and Afod at 3 doses. PDX tumors (LU-01-0032) were implanted at 4 different locations in peritoneal cavity, and high concentrated fibrinogen enriched a1at thrombin and Afod or a control agent was applied to peritoneum before and after tumor implantation.

Mice were palpated for tumors 2 weeks after implantation. The ratio of palpable tumors observed in each group was recorded. High concentrated fibrinogen enriched a1at thrombin and Afod treatment inhibited the tumor growth as shown by the delayed appearance of palpable tumors and decreased tumor incidence. Four weeks after implantation, 9 out of 13 mice in vehicle control group showed palpable tumors, while only less than 5 palpable tumors were found in each high concentrated fibrinogen enriched a1at thrombin and Afod-treated group (Table 2).

Forty-five days after implantation, the mice were sacrificed and tumors were dissected and weighed. After sacrificing the mice, tumors were found in all the mice in vehicle control group, while some tumors completely regressed in several high concentrated fibrinogen enriched a1at thrombin and Afod-treated mice. Tumors in vehicle control group reached more than 0.7 g on average. Conversely, tumor weights in high concentrated fibrinogen enriched a1at thrombin and Afod high, moderate and low dose groups were 0.19 g, 0.16 g and 0.16 g, respectively. Compared with the vehicle control, high concentrated fibrinogen enriched a1at thrombin and Afod demonstrated significant anti-tumor activities in lung cancer PDX model at all 3 doses. Matrigel has been commonly used to facilitate the establishment of human tumor xenografts in rodents. In this study, matrigel group also showed a significant inhibitory effect on tumor weight.

Loss of body weight, a sign of toxicity, was not seen in all high concentrated fibrinogen enriched a1at thrombin and Afod-treated groups, indicating the test agent has no/little side effects.

In summary, the results show that high concentrated fibrinogen enriched a1at thrombin and Afod at all doses significantly inhibits the growth of lung tumors in vivo while having minor effects on mice body weight. The results suggest that high concentrated fibrinogen enriched a1at thrombin and Afod is a potent anti-tumor agent in lung cancer.

### 6. 6.0 References

N/A References to both internal and external reports, documents and publications are listed in alphabetical order. Authors will not reference internal reports in preparation. May use @STD 3.0 Referencing tool. References to external documents and publications are indicated in the text by citing the author and year within brackets.

### 7. Figures

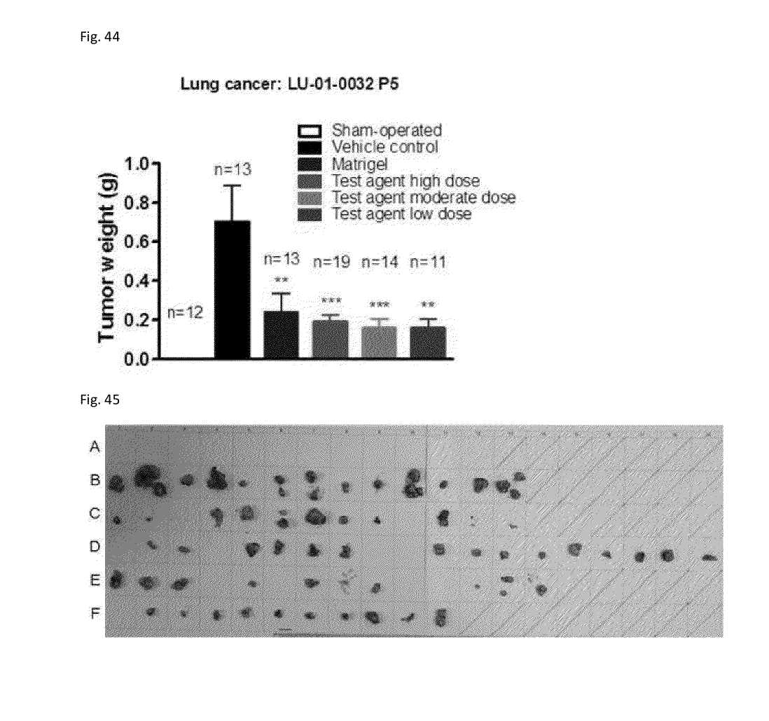

FIG. 44. Anti-Tumor Efficacy of High Concentrated Fibrinogen Enriched a1at Thrombin and Afod in PDX Model LU-01-0032.

Tumor weights from model LU-01-0032 were used. Data are expressed as mean±SEM. *<0.05, **<0.01, ***<0.001 vs vehicle group (one-way ANOVA and Dunnett's test).

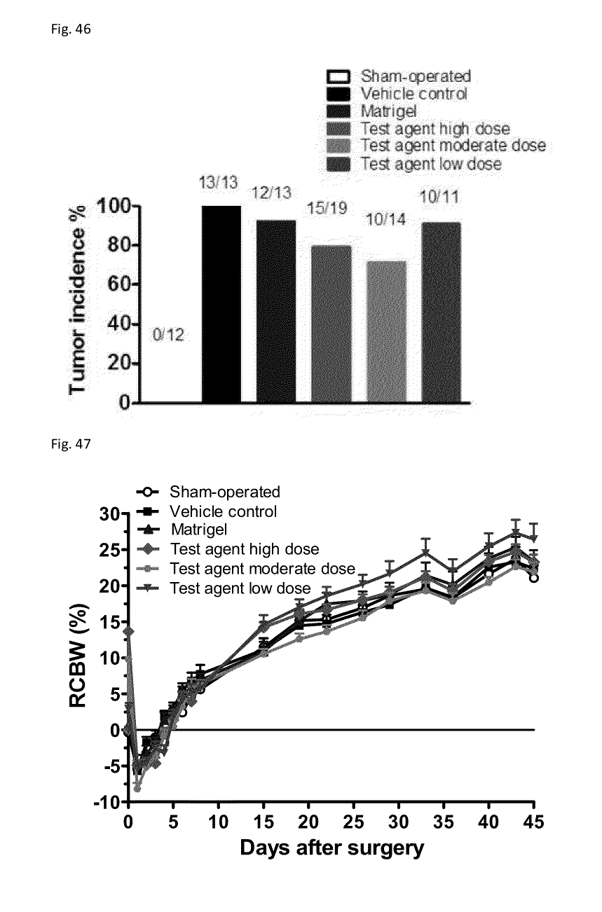

FIG. 45. Photographs of Tumors Dissected from Abdominal Cavity of Each Group.

Tumors from each mouse of model LU-01-0032 were pooled and weighed. Scale bar, 1 cm. A, sham-operated; B, vehicle control; C, matrigel; D, test agent high dose; E, test agent moderate dose; F, test agent low dose.

FIG. 46. Ratios of Mice with Palpable Tumors Observed in Each Group.

After sacrificing the mice, the tumors from each mouse of model LU-01-0032 were pooled and the ratios of mice bearing tumors in each group were recorded.

### FIG. 47. Relative Change of Body Weight (%) of Different Groups.

Data are expressed as mean±SEM. Relative change of body weight (RCBW) was calculated based on the following formula: RCBW (%)=(BWi−BW0)/BW0×100%; BWi was the body weight on the day of weighing and BW0 was the body weight before surgery.

### 8. Tables

TABLE 2
Ratios of palpable tumors observed in each group.

| | | h. Palpable tumor observed (days after surgery) | | | | | | | | | | | i. ctual incidence |
|---|---|---|---|---|---|---|---|---|---|---|---|---|---|
| g. Group | | j. 5 | k. 9 | l. 2 | m. 4 | n. 6 | o. 9 | p. 3 | q. 6 | r. 0 | s. 3 | t. 5 | at the end point |
| u. 1 | v. Sham- | w./ | x./ | y./ | z./ | aa./ | bb./ | cc./ | dd./ | ee./ | ff./ | gg./ | hh./ |
| | operated | 12 | 12 | 12 | 12 | 12 | 12 | 12 | 12 | 12 | 12 | 12 | 12 |
| ii. 2 | jj. vehicle | kk./ | ll./ | mm./ | nn./ | oo./ | pp./ | qq./ | rr./ | ss./ | tt./ | uu./ | vv./ |
| | control | 13 | 13 | 13 | 13 | 13 | 13 | 13 | 13 | 13 | 0/13 | 0/13 | 3/13 |
| ww. 3 | xx. positive | yy./ | zz./ | aaa./ | bbb./ | ccc./ | ddd./ | eee./ | fff./ | ggg./ | hhh./ | iii./ | jjj./ |
| | control | 13 | 13 | 13 | 13 | 13 | 13 | 13 | 13 | 13 | 13 | 13 | 2/13 |
| kkk. 4 | lll. high | mmm./ | nnn./ | ooo./ | ppp./ | qqq./ | rrr./ | sss./ | ttt./ | uuu./ | vvv./ | www./ | xxx./ |
| | dose | 19 | 19 | 19 | 19 | 19 | 19 | 19 | 19 | 19 | 19 | 19 | 5/19 |
| | of test agent | | | | | | | | | | | | |
| yyy. 5 | zzz. moderate | aaaa./ | bbbb./ | cccc./ | dddd./ | eeee./ | ffff./ | gggg./ | hhhh./ | iiii./ | jjjj./ | kkkk./ | llll./ |
| | dose of test agent | 14 | 14 | 14 | 14 | 14 | 14 | 14 | 14 | 14 | 14 | 14 | 0/14 |
| mmmm. | nnnn. low | oooo./ | pppp./ | qqqq./ | rrrr./ | ssss./ | tttt./ | uuuu./ | vvvv./ | www [symbol] / | xxxx./ | yyyy./ | zzzz./ |
| | dose of test agent | 11 | 11 | 11 | 11 | 11 | 11 | 11 | 11 | 11 | 11 | 11 | 0/11 |

aaaaa.
bbbbb. Mice were palpated for tumors at 15, 19, 22, 24, 26, 29, 33, 36, 40, 43, and 45 days after implantation. The ratios of palpable tumors observed in each group were recorded. [symbol]
indicates data missing or illegible when filed

TABLE 3
Relative change of body weight (%) of different groups.

| | | [symbol] | | | | | | | | | |
|---|---|---|---|---|---|---|---|---|---|---|---|
| | | 0 | 1 | 2 | 3 | 4 | 5 | 6 | 7 | 8 | 15 |
| | | RCBW | RCBW | RCBW | RCBW | RCBW | RCBW | RCBW | RCBW | RCBW | RCBW |
| Group | | (%) | (%) | (%) | (%) | (%) | (%) | (%) | (%) | (%) | (%) |
| Sham- | Mean | −0.35 | −5.43 | −4.13 | −1.00 | −1.68 | 1.68 | 2.41 | 4.73 | 5.62 | 11.48 |
| operated | SD | 2.07 | 2.95 | 3.73 | 3.23 | 4.48 | 4.41 | 5.11 | 4.29 | 5.11 | 4.30 |
| group | SEM | 0.60 | 0.85 | 1.08 | 0.93 | 1.29 | 1.27 | 1.48 | 1.24 | 1.47 | 1.24 |
| Vehicle | Mean | −0.20 | −5.68 | −1.74 | −1.45 | 1.81 | 2.96 | 5.44 | 6.79 | 7.73 | 11.14 |
| control | SD | 0.71 | 3.19 | 2.83 | 2.41 | 3.03 | 3.03 | 3.78 | 4.18 | 4.57 | 5.56 |
| group | SEM | 0.20 | 0.88 | 0.78 | 0.67 | 0.84 | 0.84 | 1.05 | 1.16 | 1.27 | 1.54 |
| Matrigel | Mean | 0.52 | −4.73 | −3.28 | −1.98 | 1.38 | 2.32 | 5.12 | 5.72 | 6.87 | 10.87 |
| group | SD | 0.70 | 4.50 | 3.91 | 3.56 | 3.72 | 3.91 | 3.24 | 3.14 | 3.48 | 4.92 |
| | SEM | 0.19 | 1.25 | 1.08 | 0.99 | 1.03 | 1.08 | 0.90 | 0.87 | 0.96 | 1.37 |
| Test | Mean | 13.64 | −4.90 | −4.88 | −4.65 | −0.53 | 1.39 | 4.29 | 3.94 | 6.13 | 14.21 |
| agent | SD | 1.28 | 2.95 | 4.08 | 3.45 | 3.59 | 4.07 | 3.86 | 3.85 | 3.28 | 3.10 |
| high | SEM | 0.29 | 0.68 | 0.94 | 0.79 | 0.82 | 0.93 | 0.88 | 0.89 | 0.75 | 0.71 |
| dose | | | | | | | | | | | |
| Test | Mean | 9.78 | −8.17 | −5.04 | −3.71 | −0.87 | 0.47 | 3.22 | 5.98 | 6.27 | 10.52 |
| agent | SD | 0.87 | 3.06 | 3.70 | 2.82 | 3.32 | 2.82 | 3.03 | 4.07 | 2.25 | 2.65 |
| moderate | SEM | 0.23 | 0.82 | 0.99 | 0.75 | 0.89 | 0.75 | 0.81 | 1.09 | 0.60 | 0.71 |
| dose | | | | | | | | | | | |
| Test | Mean | 2.92 | −5.28 | −4.58 | −2.59 | −3.22 | 1.76 | 4.16 | 5.27 | 5.66 | 14.58 |
| agent | SD | 2.88 | 2.48 | 2.73 | 3.47 | 3.97 | 3.40 | 4.03 | 3.53 | 3.69 | 4.36 |
| low | SEM | 0.80 | 0.69 | 0.76 | 0.96 | 1.10 | 1.03 | 1.22 | 1.06 | 1.11 | 1.31 |
| dose | | | | | | | | | | | |

| | | [symbol] | | | | | | | | |
|---|---|---|---|---|---|---|---|---|---|---|
| | | 19 | 22 | 26 | 29 | 33 | 36 | 40 | 43 | 45 |
| | | RCBW | RCBW | RCBW | RCBW | RCBW | RCBW | RCBW | RCBW | RCBW |
| Group | | (%) | (%) | (%) | (%) | (%) | (%) | (%) | (%) | (%) |
| Sham- | Mean | 15.15 | 15.32 | 16.90 | 18.60 | 19.51 | 18.31 | 21.64 | 23.04 | 21.07 |
| operated | SD | 4.11 | 4.07 | 4.56 | 4.35 | 4.41 | 3.60 | 3.45 | 3.67 | 4.32 |
| group | SEM | 1.19 | 1.17 | 1.32 | 1.25 | 1.27 | 1.04 | 0.99 | 1.06 | 1.25 |
| Vehicle | Mean | 14.45 | 14.73 | 16.29 | 17.34 | 19.71 | 18.37 | 22.55 | 23.29 | 22.38 |
| control | SD | 4.47 | 4.45 | 3.63 | 4.92 | 5.70 | 5.49 | 6.93 | 7.50 | 6.86 |
| group | SEM | 1.24 | 1.23 | 1.01 | 1.36 | 1.58 | 1.52 | 1.92 | 2.08 | 1.90 |
| Matrigel | Mean | 15.11 | 17.48 | 17.91 | 18.76 | 21.42 | 20.11 | 23.76 | 25.31 | 23.34 |
| group | SD | 5.03 | 5.55 | 4.66 | 5.92 | 6.37 | 6.68 | 5.84 | 5.28 | 5.64 |
| | SEM | 1.40 | 1.54 | 1.29 | 1.64 | 1.77 | 1.85 | 1.62 | 1.47 | 1.56 |
| Test | Mean | 16.06 | 16.63 | 18.03 | 19.06 | 21.19 | 19.26 | 23.34 | 24.67 | 23.26 |
| agent | SD | 2.77 | 3.39 | 3.42 | 3.31 | 3.63 | 4.03 | 4.08 | 4.66 | 4.64 |
| high | SEM | 0.64 | 0.78 | 0.78 | 0.76 | 0.83 | 0.92 | 0.94 | 1.07 | 1.06 |
| dose | | | | | | | | | | |
| Test | Mean | 12.58 | 13.61 | 15.53 | 17.87 | 19.31 | 17.88 | 20.45 | 22.62 | 21.93 |
| agent | SD | 2.90 | 3.46 | 3.87 | 4.27 | 4.31 | 4.01 | 2.98 | 3.72 | 4.80 |
| moderate | SEM | 0.78 | 0.93 | 1.03 | 1.14 | 1.15 | 1.07 | 0.80 | 1.00 | 1.28 |
| dose | | | | | | | | | | |
| Test | Mean | 16.99 | 18.59 | 20.19 | 21.65 | 24.49 | 21.97 | 25.45 | 27.30 | 26.43 |
| agent | SD | 3.75 | 4.06 | 4.34 | 5.72 | 6.59 | 5.54 | 5.93 | 6.01 | 7.15 |
| low | SEM | 1.13 | 1.22 | 1.31 | 1.73 | 1.99 | 1.67 | 1.79 | 1.81 | 2.15 |
| dose | | | | | | | | | | |

Relative change of body weight (RCBW) was calculated based on the following formula: RCBW (%) = (BWi − BW0)/BW0 × 100%; BWi was the body weight on the day of weighing and BW0 was the body weight before surgery.

### RAAS

b.

### Title: In Vivo Test of Efficacy of FS in the Treatment of BEL-7404 Peritoneal Implantation Model

Description: BEL-7404 peritoneal Implantation hepatic cancer model was used to evaluate the anti-cancer efficacy of the FS at 2 ml/mouse. The results showed that FS had inhibition on tumor growth.
Subject: FS, BEL-7404, hepatic cancer

### Project ID: RAAS-20130425

Summary

BEL-7404 peritoneal Implantation hepatic cancer model was used to evaluate the anti-cancer efficacy of FS at 2 ml/mouse. On day 21 after implantation, all mice in vehicle group and positive group exhibited palpable tumors, while no mice in FS group exhibited any palpable tumors. On day 28 after implantation, the tumor in vehicle group reached 0.88 g, while FS group was only 0.06 g. On day 52 after implantation, Five mice treated with FS did not exhibit any palpable tumor. The results showed that FS had inhibition on tumor growth.

No body weight loss and toxicity were found in FS-treated groups, which showed FS had no side effect.

### 9. 1. Details of Facility, Personnel and Data Location

| | |
|---|---|
| Sponsor: | RAAS |
| Test Facility: | WuXi AppTec Animal facility in 90 Delin Road, |
| | Waigaoqiao Free Trade Zone, Shanghai 200131, |
| | P.R.China. |
| Date of Work: | Commenced: Feb. 20, 2013 |
| | Completed: Jul. 13, 2013 |
| Personnel Involved: | Qingyang Gu Study director Ph.D |
| | Yunbiao Yan Scientist MS |
| | Bei Wang Scientist MS |
| Study Director/ | Qingyang Gu Director Ph.D |
| Senior Scientist: | |

### Location of Raw Data, Original Protocols, Experimental Details and Report

The studies described in this report were carried out on behalf of RAAS at external laboratories:

All raw data, protocols and experimental details pertaining to these studies and the original of the report will be held in the Archive of WuXi AppTec in 90 Delin Road, Waigaogiao Free Trade Zone, Shanghai 200131, P.R.China.

### 2. Introduction

The objective of the research is to evaluate the in vivo efficacy of FS in the treatment of a hepatic cancer model.

All the experiments were conducted in the AAALAC-accrediated animal facility in compliance with the protocol approved by the Institutional Animal Care and Use Committee (IACUC).

### 3. Methods

### 3.1. Experimental Preparations

### 3.1.1. Animal Preparation

Female Balb/c nude mice, with a body weight of approximately 20 grams, were obtained from an approved vendor (Shanghai BK Laboratory Animal Co., LTD., Shanghai, China).

### Acclimation/Quarantine:

Upon arrival, animals were assessed as to their general health by a member of a veterinary staff or authorized personnel. Animals were acclimated for at least 3 days (upon arrival at the experiment room) before being used for the study.

### Animal Husbandry:

Animals were housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Shanghai BK Laboratory Animal Co., LTD., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcome of the study.

### 3.1.2. Cell Culture:

The BEL-7404 tumor cells were maintained in vitro as a monolayer culture in RPMI 1640 medium supplemented with 10% heat inactivated fetal bovine serum, 100 U/ml penicillin and 100 μg/ml streptomycin, and L-glutamine (2 mM) at 37° C. in an atmosphere of 5% CO₂ in air. The tumor cells were routinely subcultured twice weekly by trypsin-EDTA treatment. The cells growing in an exponential growth phase were harvested and counted for tumor inoculation. When the average tumor volume reached 1000 mm³, sacrificed the tumor bearing mice and removed the tumor for orthotopic implantation.

### 3.1.3. Formulation

FS were provide by RAAS and prepared by RAAS scientist during experiment before use. Matrigel (BD Biosciences; cat. #356234)

### 3.2. Experimental Protocol

### 3.2.1. Establishment of Xenograft Model and Treatment

### Grouping and Treatment

Nude mice were assigned to 6 different groups with 3 mice/group and each group received different treatment as shown in Table 1.

TABLE 1
Grouping and the treatment

| Group | Treatment | N | Remarks |
|---|---|---|---|
| 1 | Vehicle | 3 | Implant a tumor fragment of 40 mm³ into the abdominal |
| | control | | cavity. Close body with sutures |
| 2 | Positive | 3 | Embed a tumor fragment of 40 mm³ in Matrigel. |
| | control | | Implant the tumor fragment into abdominal cavity. |
| | | | Close body with sutures |
| 3 | 3- FS | 3 | Spray FS to cover the entire peritoneum and the |
| | (2 ml) + | | internal organs. Implant a tumor fragment of 40 mm³ |
| | sorafenib | | into abdominal cavity, spread some sorafenib on the |
| | | | tumor. Spray FS to cover the tumor. Close body with |
| | | | sutures |
| 4 | FS alone | 3 | Spray FS to cover the entire peritoneum and the |
| | (2 ml) | | internal organs. Implant a tumor fragment of 40 mm³ |
| | | | into abdominal cavity. Spray FS to cover the tumor. |
| | | | Close body with sutures |
| 5 | FS alone | 3 | Spray FS to cover the entire peritoneum and the |
| | (2 ml) | | internal organs. Implant a tumor fragment of 40 mm³ |
| | | | into abdominal cavity. Spray FS to cover the tumors. |
| | | | Close body with sutures |
| 6 | FS + oral | 3 | Spray FS to cover the entire peritoneum and the |
| | application | | internal organs. Implant a tumor fragment of 40 mm³ |
| | (2 ml) | | into abdominal cavity. Spray FS to cover the tumors. |
| | | | Close body with sutures. After implantation, the mice |
| | | | were treated according to table2 |
| Total | | 18 | |

TABLE 2
treatment schedule in group 6

| Medicine | Concentration | Route | Dosage | Schedule |
|---|---|---|---|---|
| kh afcc | NA | Drink | NA | 22 h on 2 h off (D 0-D 8) |
| kh afcc | NA | PO | 0.4 ml | BID (D 9-D 31) |
| KH-R1 | NA | Drink | NA | 24 h (D 32-D 42) |
| 5bp121 | NA | IP | 0.5 ml | QD (D 43-D 54) |

The doses of group6 was changed during the experiment as requested by the sponsor

### Experiment Procedures

P. Establishment of cell line xenograft tumor model in female Balb/c nude mice: cells were injected at 3×10⁶ cells per animal subcutaneously.
Q. When the tumor reached about 1000 mm³, sacrifice these animals, removed tumor for peritoneal implantation.
R. Fifteen mice were anesthetized by i.p. injection of sodium pentobarbital at 60-70 mg/kg. The animal skin was sterilized with ethanol solution. Then the body wall was opened and the peritoneal surface was exposed.
S. The test agent was then applied on the peritoneal surface, including both sides. The amount of the test agent was listed on the above table.
T. Tumor fragment was implanted into the peritoneum. The test agent acted as a glue to hold the fragment.
U. After the fibrin membrane formed completely, the peritoneal cavity was closed and sutured.
V. In the positive control group, tumor fragment was embedded in Matrigel.
W. Body weight was measured upon completion of surgery.
X. Postoperative cares followed protocol SOP-BEO-0016-1.0.
Y. Group 1, 2 and 4 were sacrificed 4 weeks after implantation with tumor growing in all vehicle and positive control. Group 3, 5 and 6 were for long term study for 54 days.
Z. During the period of the experiment, health conditions of mice were observed daily. Body weight of mice was monitored twice per week.
AA. Tumor sizes were measured twice per week when tumor could be measured. Tumor volumes (mm³) were obtained by using the following formula: volume=(W²×L)/2 (W, width; L, length in mm of the tumor)
BB. Mice, which showed a significant loss of body weight (>20%), or which were unable to eat or drink, or exhibit ulceration on the skin/tumor, or the tumor size reaches 2,000 mm³, were euthanized immediately to minimize the pain and distress. Such actions need to notify the sponsor within 24 hrs (48 hrs during the weekends).

### 3.2.2. Evaluation of the Anti-Tumor Activity

Health conditions of mice were observed daily. Body weights were measured twice per week during the treatment. The ratio of palpable tumors observed in each group was recorded. Group 1, 2 and 4 were sacrificed 4 weeks after implantation with tumor growing in all vehicle or positive control. Group 3, 5 and 6 were for long term study for 54 days. Each tumor and FS was removed and weighed.

### 3.3. Drugs and Materials

FS was provided by RAAS; Matrigel was from BD Biosciences (San Jose, Calif., cat. #356234). Digital caliper was from Sylvac, Switzerland.

### 3.4. Data Analysis

### 3.4.1. Ratios of Palpable Tumors Observed in Different Groups

Record the palpable tumors of each mouse observed as an indicator of efficacy

### 3.4.2. Tumor Weight

Group 1, 2 and 4 were sacrificed 4 weeks after implantation with tumor growing in all vehicle or positive control. Group 3, 5 and 6 were for long term study for 54 days. Each tumor was removed and weighted.

### 3.4.3. Relative Change of Body Weight (Rcbw

Relative change of body weight (RCBW) was calculated based on the following formula: RCBW (%)=(BWi−BW0)/BW0×100%; BWi was the body weight on the day of weighing and BW0 was the body weight before surgery.

### 3.4.4. Statistical Analysis

Data was expressed as mean±S.E.;

### 4. Results

### 4.1. Ratios of Palpable Tumors Observed in Different Groups

On day 21 after implantation, all mice in vehicle group and all mice in positive group exhibited palpable tumor, while no mice in FS groups exhibited any palpable tumors. On 52 days after implantation, five mice treated with FS did not exhibit any palpable tumor. The summary of ratios of palpable tumors observed in different groups was shown in table 3.

### 4.2. Tumor Weight

On day 28 after implantation, the tumor weight of vehicle group, positive group and FS group were 0.88 g, 1.02 g and 0.06 respectively, the tumor weight was shown in table 4.

### 4.3. Body Weight

Loss of body weight or a sign of toxicity was not found in FS-treated groups.

The effect on body weight was shown in table 5.

### 5. Discussion

EL-7404 peritoneal Implantation hepatic cancer model was used to evaluate the anti-cancer efficacy of FS at 2 ml/mouse. On day 21 after implantation, all mice in vehicle group and positive group exhibited palpable tumors, while no mice in FS group exhibited any palpable tumors. On day 28 after implantation, the tumor in vehicle group reached 0.88 g, while FS group was only 0.06 g. On day 52 after implantation, Five mice treated with FS did not exhibit any palpable tumor. The results showed that FS had inhibition on tumor growth.

No body weight loss and toxicity were found in FS-treated groups, which showed FS had no side effect.

### 6. References

N/A

### Tables

TABLE 3
Ratios of palpable tumors observed in different groups

| | Days after surgery | | | | | | | | | |
|---|---|---|---|---|---|---|---|---|---|---|
| Group | 21 | 24 | 28 | 31 | 35 | 38 | 42 | 45 | 49 | 52 |
| Vehicle | 3/3 | 3/3 | 3/3 | sacrificed | sacrificed | sacrificed | sacrificed | sacrificed | sacrificed | sacrificed |
| Positive | 3/3 | 3/3 | 3/3 | sacrificed | sacrificed | sacrificed | sacrificed | sacrificed | sacrificed | sacrificed |
| FS (2 ml) + | 0/3 | 0/3 | 0/3 | 0/3 | 0/3 | 0/3 | 0/3 | 0/3 | 0/3 | 0/3 |
| sorafenib | | | | | | | | | | |
| FS (2 ml) | 0/3 | 0/3 | 1/3 | sacrificed | sacrificed | sacrificed | sacrificed | sacrificed | sacrificed | sacrificed |
| FS (2 ml) | 0/3 | 0/3 | 0/3 | 0/3 | 0/3 | 1/3 | 1/3 | 1/3 | 1/3 | 1/3 |
| FS (2 ml) + | 0/3 | 0/3 | 0/3 | 1/3 | 2/3 | 2/3 | 2/3 | 2/3 | 2/3 | 2/3 |
| oral | | | | | | | | | | |
| application | | | | | | | | | | |

TABLE 4
The summary of FS weight and tumor weight

| | Pre- | | | | | |
|---|---|---|---|---|---|---|
| | test | | | Days after | FS Weight | Tumor Weight |
| Group | Mice # | Mice # | status | implantation | (g) | (g) |
| Vehicle | 1 | 1-1 | sacrificed | 28 | N/A | 0.87 |
| | 2 | 1-2 | sacrificed | 28 | N/A | 0.68 |
| | 3 | 1-3 | sacrificed | 28 | N/A | 1.09 |
| Positive | 4 | 2-1 | sacrificed | 28 | N/A | 3.56 |
| | 5 | 2-2 | sacrificed | 28 | N/A | 1.15 |
| | 6 | 2-3 | sacrificed | 28 | N/A | 1.35 |
| FS (2 ml) + | 7 | 3-1 | died | 22 | 1.25 | 0.00 |
| sorafenib | 8 | 3-2 | sacrificed | 54 | 1.22 | 0.00 |
| | 9 | 3-3 | sacrificed | 54 | 0.92 | 0.00 |
| FS (2 ml) | 10 | 4-1 | sacrificed | 28 | 1.06 | 0.10 |
| | 11 | 4-2 | sacrificed | 28 | 0.72 | 0.00 |
| | 12 | 4-3 | sacrificed | 28 | 0.78 | 0.09 |
| FS (2 ml) | 13 | 5-1 | sacrificed | 54 | 0.81 | 0.75 |
| | 14 | 5-2 | sacrificed | 54 | 0.80 | 0.00 |
| | 15 | 5-3 | sacrificed | 54 | 0.81 | 0.00 |
| FS (2 ml) + | 16 | 6-1 | sacrificed | 54 | 1.08 | 2.16 |
| oral | 18 | 6-3 | sacrificed | 54 | 0.95 | 0.00 |
| application | 19 | 6-2 | died | 51 | 0.91 | 3.03 |

TABLE 5
Relative change of body weight (%) of different groups

| | | Days after surgery | | | | | | | | | | |
|---|---|---|---|---|---|---|---|---|---|---|---|---|
| | | 0 | 0 | 2 | 3 | 4 | 5 | 6 | 7 | 11 | 14 | 21 |
| | | RCBW | RCBW | RCBW | RCBW | RCBW | RCBW | RCBW | RCBW | RCBW | RCBW | RCBW |
| Compounds | Mice # | (%) | (%) | (%) | (%) | (%) | (%) | (%) | (%) | (%) | (%) | (%) |
| Group | 1 | 0.00 | — | 4.16 | 4.42 | 8.27 | 7.54 | 9.62 | 6.19 | 15.0 | 12.4 | 18.9 |
| 1- | 2 | 0.00 | — | 0.58 | 1.38 | 3.13 | 6.31 | 6.15 | 4.24 | 10.9 | 10.2 | 13.5 |
| | 3 | 0.00 | 0.58 | — | — | 0.38 | 0.96 | 3.08 | 0.67 | 6.50 | 6.45 | 6.26 |
| | Mean | 0.00 | — | 1.08 | 1.29 | 3.93 | 4.94 | 6.28 | 3.70 | 10.8 | 9.72 | 12.9 |
| | SD | 0.00 | 0.77 | 2.86 | 3.17 | 4.00 | 3.50 | 3.27 | 2.80 | 4.27 | 3.03 | 6.36 |
| Group | 4 | 0.00 | — | — | — | — | — | — | 0.80 | 6.94 | 9.19 | 15.9 |
| 2- | 5 | 0.00 | 0.21 | — | 5.71 | 7.54 | 7.33 | 9.11 | 11.6 | 14.9 | 15.1 | 22.6 |
| | 6 | 0.00 | — | 0.58 | 0.74 | 3.28 | 2.01 | 2.38 | 2.22 | 10.1 | 7.29 | 8.46 |
| | Mean | 0.00 | — | — | 0.32 | 2.82 | 3.05 | 3.60 | 4.88 | 10.6 | 10.5 | 15.6 |
| | SD | 0.00 | 1.05 | 2.61 | 5.61 | 4.96 | 3.87 | 5.02 | 5.89 | 4.05 | 4.12 | 7.09 |
| Group | 7 | 0.00 | 13.6 | — | — | — | 3.04 | 5.86 | 3.14 | 9.22 | 9.59 | 15.6 |
| 3-FS | 8 | 0.00 | 14.0 | — | — | 0.51 | 5.17 | 6.64 | 4.67 | 6.09 | 18.9 | 23.2 |
| | 9 | 0.00 | 10.2 | 0.00 | 3.67 | 3.51 | 6.06 | 5.42 | 6.54 | 21.1 | 15.0 | 41.7 |
| | Mean | 0.00 | 12.6 | — | — | 1.32 | 4.76 | 5.97 | 4.78 | 12.1 | 14.5 | 26.8 |
| | SD | 0.00 | 2.06 | 1.90 | 3.37 | 1.91 | 1.55 | 0.62 | 1.70 | 7.95 | 4.71 | 13.4 |
| Group | 10 | 0.00 | 13.7 | 2.57 | 6.00 | 9.38 | 13.5 | 19.8 | 16.9 | 22.0 | 23.9 | 29.4 |
| 4-FS | 11 | 0.00 | 12.2 | — | — | — | 4.78 | 10.7 | 13.3 | 21.0 | 22.4 | 26.8 |
| | 12 | 0.00 | 6.58 | — | 2.14 | 5.43 | 10.9 | 12.5 | 10.7 | 14.5 | 14.8 | 19.9 |
| | Mean | 0.00 | 10.8 | — | 2.49 | 4.90 | 9.75 | 14.3 | 13.6 | 19.2 | 20.4 | 25.4 |
| | SD | 0.00 | 3.76 | 3.98 | 3.36 | 4.76 | 4.51 | 4.82 | 3.15 | 4.10 | 4.83 | 4.94 |
| Group | 13 | 0.00 | 9.04 | — | 3.18 | 5.91 | 12.2 | 13.7 | 12.6 | 21.5 | 26.8 | 31.3 |
| 5-FS | 14 | 0.00 | 10.3 | 1.49 | 2.93 | 6.37 | 8.42 | 9.39 | 9.45 | 16.7 | 19.0 | 28.2 |
| | 15 | 0.00 | 12.6 | 1.80 | 5.51 | 6.85 | 9.17 | 9.02 | 7.21 | 15.1 | 19.4 | 21.1 |
| | Mean | 0.00 | 10.7 | 0.73 | 3.87 | 6.38 | 9.96 | 10.7 | 9.78 | 17.8 | 21.7 | 26.9 |
| | SD | 0.00 | 1.84 | 1.60 | 1.42 | 0.47 | 2.05 | 2.66 | 2.75 | 3.34 | 4.39 | 5.20 |
| Group | 16 | 0.00 | 9.97 | — | 2.83 | 1.31 | 4.41 | — | 3.41 | 10.1 | 10.8 | 17.8 |
| 6-FS | 18 | 0.00 | 10.3 | 6.47 | 7.40 | 1.76 | 7.16 | — | 1.76 | 11.5 | 11.0 | 14.3 |
| | 19 | 0.00 | NA | NA | 2.62 | — | — | — | — | 1.26 | 0.73 | 5.30 |
| | Mean | 0.00 | 11.2 | 1.01 | 4.29 | — | 2.40 | — | — | 7.65 | 7.52 | 12.4 |
| | SD | 0.00 | 1.88 | 4.93 | 2.70 | 3.35 | 6.01 | 7.07 | 5.89 | 5.58 | 5.88 | 6.47 |

| | | Days after surgery | | | | | | | | |
|---|---|---|---|---|---|---|---|---|---|---|
| | | 24 | 28 | 31 | 35 | 38 | 42 | 45 | 49 | 52 |
| | | RCBW | RCBW | RCBW | RCBW | RCBW | RCBW | RCBW | RCBW | RCBW |
| Compounds | Mice # | (%) | (%) | (%) | (%) | (%) | (%) | (%) | (%) | (%) |
| Group 1- | 1 | 22.3 | 26.3 | sacrifi | sacrifi | sacrifi | sacrifi | sacrifi | sacrifi | sacrifi |
| | 2 | 15.4 | 24.5 | sacrifi | sacrifi | sacrifi | sacrifi | sacrifi | sacrifi | sacrifi |
| | 3 | 6.26 | 8.33 | sacrifi | sacrifi | sacrifi | sacrifi | sacrifi | sacrifi | sacrifi |
| | Mean | 14.6 | 19.7 | | | | | | | |
| | SD | 8.05 | 9.92 | | | | | | | |
| Group 2- | 4 | 22.7 | 32.5 | sacrifi | sacrifi | sacrifi | sacrifi | sacrifi | sacrifi | sacrifi |
| | 5 | 23.7 | 30.1 | sacrifi | sacrifi | sacrifi | sacrifi | sacrifi | sacrifi | sacrifi |
| | 6 | 15.0 | 24.0 | sacrifi | sacrifi | sacrifi | sacrifi | sacrifi | sacrifi | sacrifi |
| | Mean | 20.4 | 28.9 | | | | | | | |
| | SD | 4.77 | 4.39 | | | | | | | |
| Group 3- | 7 | died | died | died | died | died | died | died | died | died |
| FS | 8 | 25.0 | 24.3 | 30.63 | 28.50 | 30.48 | 29.11 | 26.27 | 33.11 | 29.82 |
| | 9 | 24.8 | 25.9 | 34.54 | 32.73 | 33.21 | 33.48 | 31.03 | 36.66 | 35.18 |
| | Mean | 24.9 | 25.1 | 32.58 | 30.62 | 31.84 | 31.29 | 28.65 | 34.89 | 32.50 |
| | SD | 0.13 | 1.09 | 2.76 | 2.99 | 1.93 | 3.09 | 3.37 | 2.51 | 3.79 |
| Group | 10 | 31.1 | 32.2 | sacrifi | sacrifi | sacrifi | sacrifi | sacrifi | sacrifi | sacrifi |
| 4-FS | 11 | 25.7 | 24.6 | sacrifi | sacrifi | sacrifi | sacrifi | sacrifi | sacrifi | sacrifi |
| | 12 | 22.5 | 25.2 | sacrifi | sacrifi | sacrifi | sacrifi | sacrifi | sacrifi | sacrifi |
| | Mean | 26.4 | 27.3 | | | | | | | |
| | SD | 4.33 | 4.20 | | | | | | | |
| Group | 13 | 29.1 | 32.3 | 34.66 | 37.44 | 39.97 | 40.63 | 33.80 | 43.05 | 42.14 |
| 5-FS | 14 | 22.1 | 27.1 | 25.62 | 26.49 | 29.41 | 28.18 | 28.75 | 30.49 | 26.80 |
| | 15 | 21.7 | 24.1 | 24.27 | 26.02 | 24.73 | 24.27 | 18.19 | 24.11 | 21.69 |
| | Mean | 24.3 | 27.8 | 28.18 | 29.98 | 31.37 | 31.03 | 26.91 | 32.55 | 30.21 |
| | SD | 4.13 | 4.14 | 5.65 | 6.46 | 7.81 | 8.54 | 7.97 | 9.64 | 10.64 |
| Group | 16 | 16.5 | 18.9 | 23.82 | 24.34 | 30.33 | 35.78 | 41.08 | 39.14 | 42.81 |
| 6-FS | 18 | 20.1 | 19.6 | 20.88 | 25.05 | 26.91 | 27.70 | 28.19 | 21.13 | 18.87 |
| | 19 | 3.41 | 4.78 | 5.46 | 14.33 | 22.73 | 33.18 | 41.15 | 49.08 | died |
| | Mean | 13.3 | 14.4 | 16.72 | 21.24 | 26.66 | 32.22 | 36.81 | 36.45 | 30.84 |
| | SD | 8.80 | 8.41 | 9.86 | 6.00 | 3.80 | 4.13 | 7.47 | 14.17 | 16.93 |

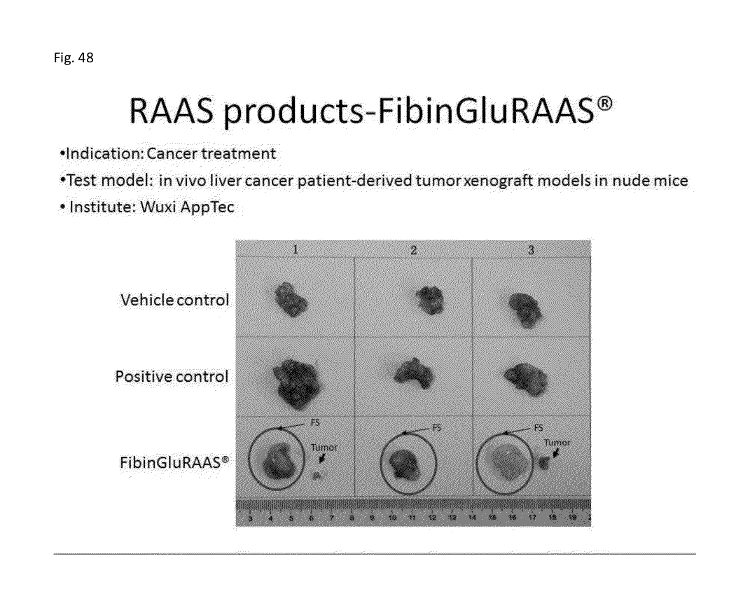

### FIG. 48

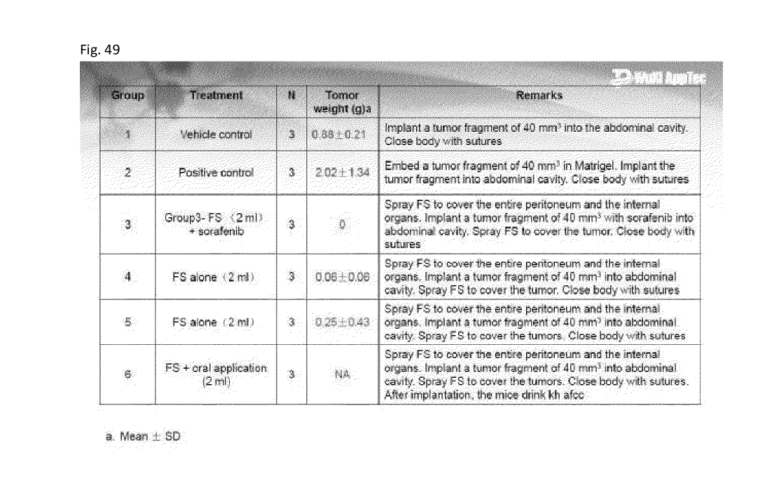

### FIG. 49

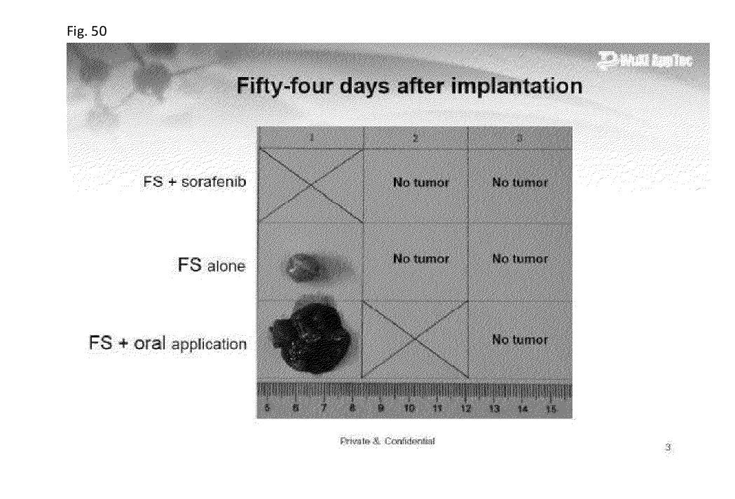

### FIG. 50

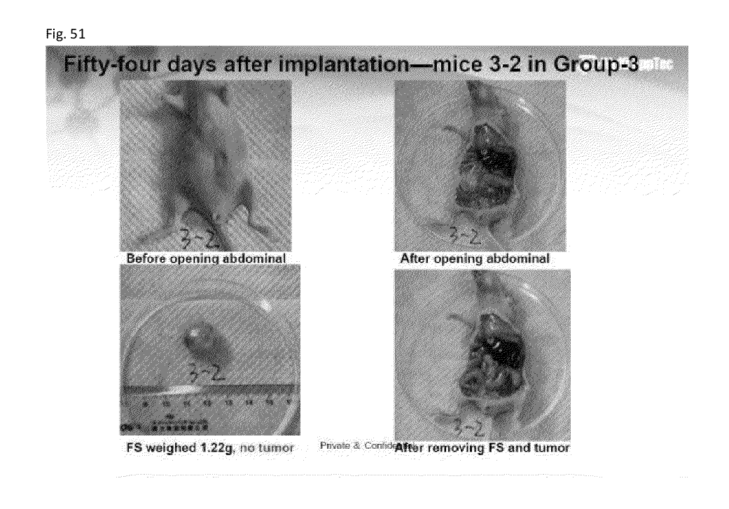

### FIG. 51

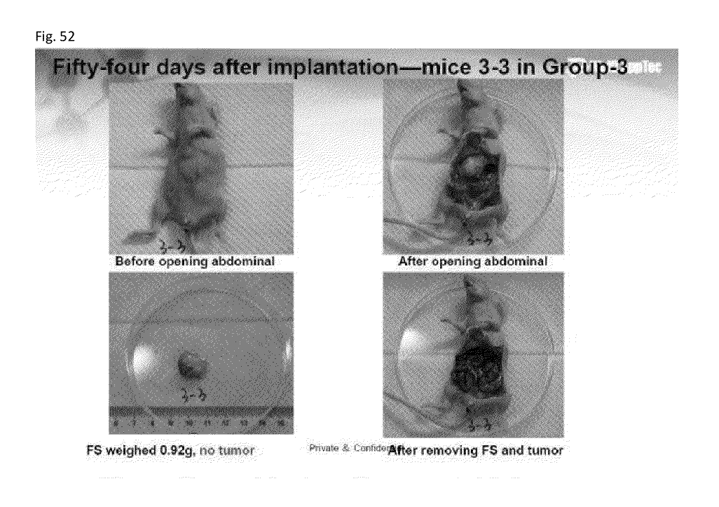

### FIG. 52

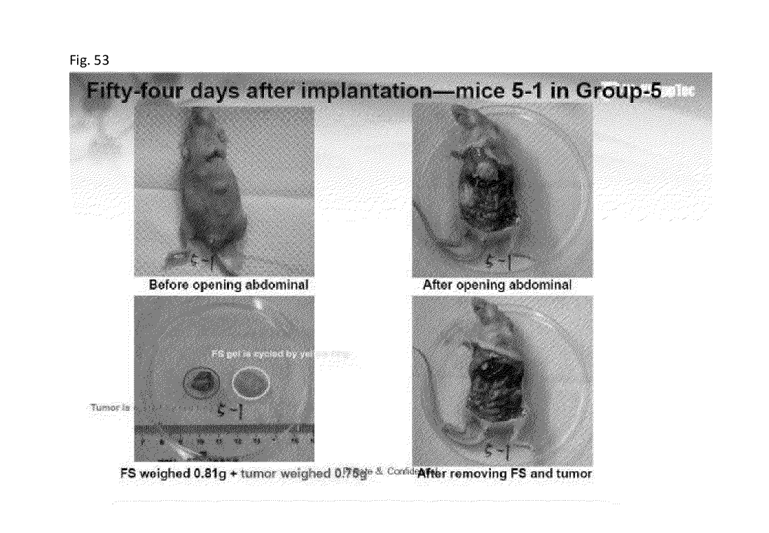

### FIG. 53

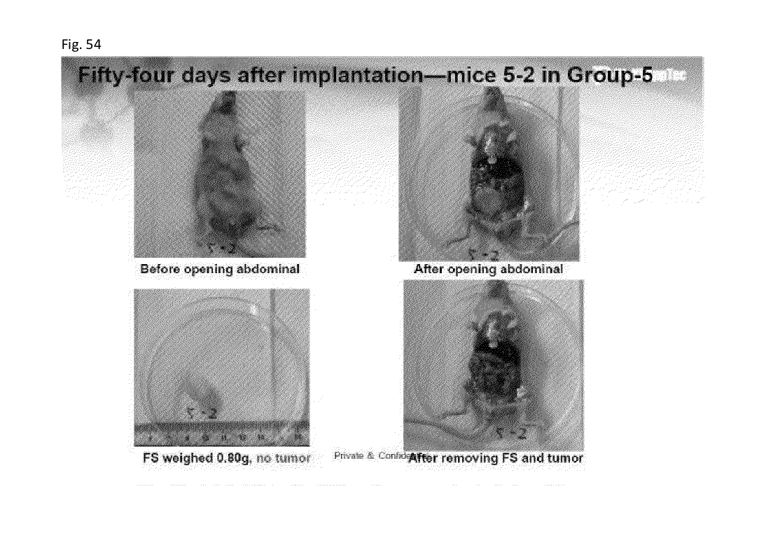

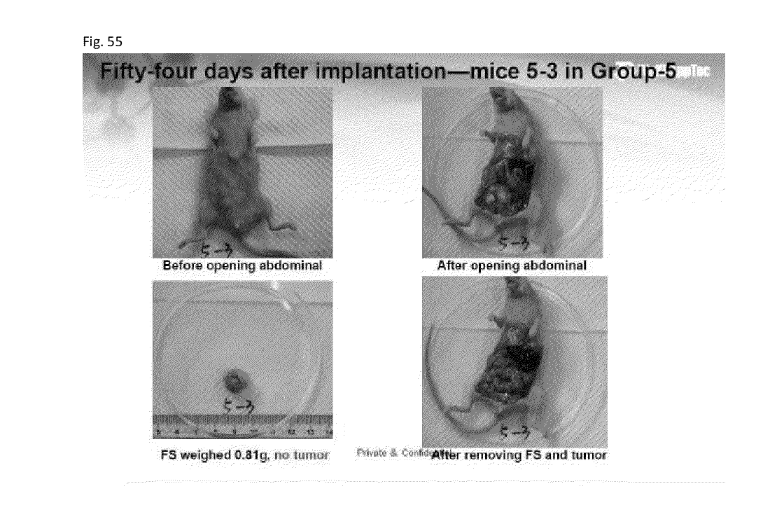

### FIG. 54

### FIG. 55

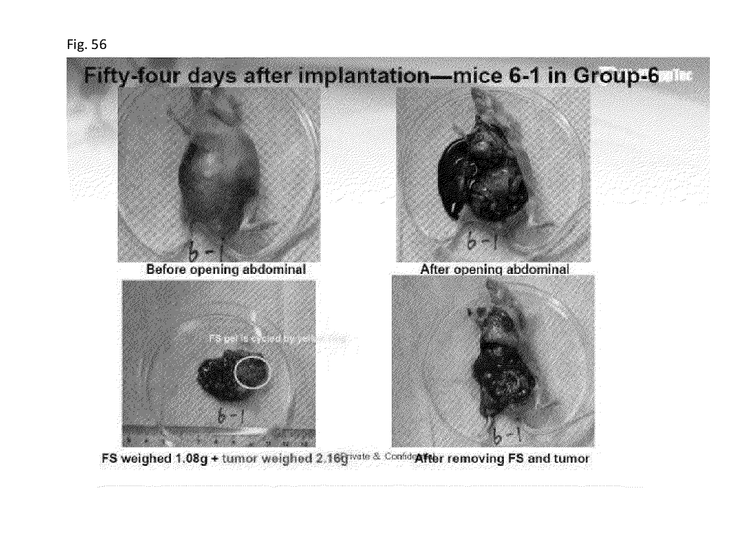

### FIG. 56

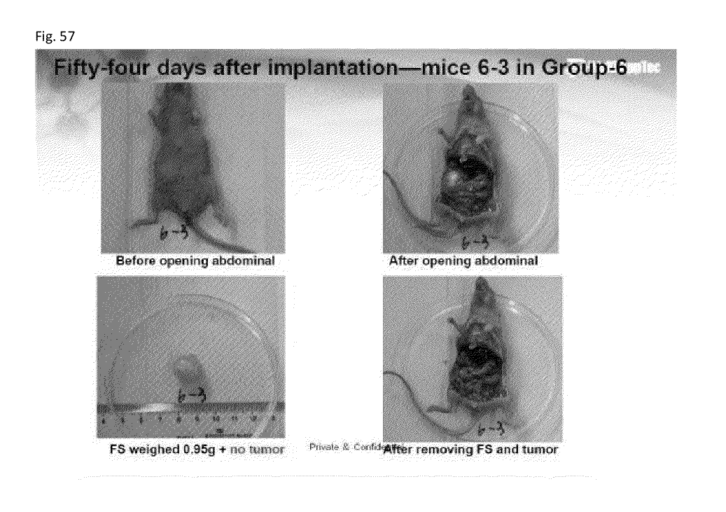

FIG. 57

1. A method of purifying fibrinogen from plasma Fraction I or from plasma cryoprecipate comprising:
a) collecting plasma cryopaste or Fraction I from human plasma;
b) dissolving the Fraction I or cryopaste acquired in step a) in a pretreatment buffer, and conducting SID virus inactivation for enveloped virus;
c) loading the treated solution from step b) to a canion chromatography;
d) using cold ethanol precipitation to purify fibrinogen from the flow through in step c);
e) loading elution buffer I to obtain factor VIII;
f) dissolving a paste generated in step d) by using Buffer II for final formulation; and
g) dialyzing and adding a stabilizer in the bulk acquired in step f).

2. The method of claim 1, wherein the Fraction I and cryopaste obtained by Cohn ethanol fractionation method.

3. The method of claim 1, wherein the pretreatment buffer includes Tris-HCl, NaCl-citrate, sucrose, and NaCl.

4. The method of claim 1, further comprising enriching and preserving high concentrated fibrinogen with fibrinolysis A1AT during purification of the high concentrated fibrinogen.

5. The method of claim 1 further comprising, improving the stability of a fibrin glue membrane by adding A1AT.

6. The method of claim 5, further comprising further enrichment of A1AT in the high concentrated fibrinogen greatly improves the stability of a fibrin glue membrane.

7. A kit comprising lyophilized or frozen high concentrated fibrinogen and thrombin, wherein the high concentrated fibrinogen is intentionally enriched and preserved with fibrinolysis inhibitor A1AT to compound a glue membrane and either not heated, or dry, wet or vapor heated up to at least 1° C. during purification of the high concentrated fibrinogen to intensify the stability and durability of the compounded glue membrane, and wherein the high concentrated fibrinogen undergoes a first step of virus inactivation for inactivating all enveloped viruses and a second step of virus activation for inactivating all non-enveloped viruses.

8. Lyophilized or frozen high concentrate fibrinogen used to compound a glue membrane, the glue membrane's diameter being less than 0.005 micrometers.

9. The kit as claimed in claim 7, wherein A1AT 5% is separately produced and added in the final bulk of the high concentrated fibrinogen.

10. The kit as claimed in claim 7, wherein A1AT is enriched and preserved during the purification process by adding pure A1AT to final bulk, increasing the stability and density of the compounded glue membrane.

11. The kit as claimed in claim 7, wherein the heating is done by the means of wet, dry, and vapor up to at least 1° C. and above preferably at least 101° C. to intensify the density of the compounded glue membrane.

12. The kit as claimed in claim 7, wherein virus inactivation for inactivating all non-enveloped viruses comprises heating up to at least 101° C.

13. The kit as claimed in claim 7, wherein the first step of virus inactivation for inactivating all enveloped viruses comprises application of a solvent detergent (S/D) TNBP and Tween 80.

14. The kit as claimed in claim 7, wherein a solvent detergent (S/D) TNBP and Tween 80 are applied for inactivating all enveloped viruses, and nanofiltration is applied for inactivating all non-enveloped viruses and all enveloped viruses in the thrombin.

15. The kit as claimed in claim 7, wherein the compounded glue membrane further comprises a compounded glue membrane mesh, the diameter of the glue membrane mesh being smaller than 10-100 μm at its largest diameter.

16. The kit as claimed in claim 7, wherein the kit is applied in preventing dissociative tumor cell pervasion in clinical operations.

17. A method of preventing dissociative tumor cell pervasion in clinical operations comprising producing a glue membrane on at least one surface area where at least one cancer tumor has been removed.

18. The method of claim 17, wherein the glue membrane is produced by applying a solution of lyophilized or frozen high concentrated fibrinogen and a solution of thrombin to the surfaces of areas where a cancer tumor has been removed.

19. The method of claim 18, wherein the solution of lyophilized or frozen high concentrated fibrinogen and the solution of thrombin are applied alternately to the surfaces of areas where a cancer tumor has been removed.

20. The method of claim 18, wherein the solution of high concentrated fibrinogen and the solution of thrombin are each applied 3 to 5 times to the surfaces of areas where a cancer tumor has been removed.

21. The method of claim 18, wherein the high concentrated fibrinogen solution and the thrombin solution are applied to compound a glue membrane with a mesh bore diameter, and wherein the mesh bore diameter is less than 10-100 micrometers at its largest diameter.

22. The method of claim 17, wherein the glue membrane prevents cancer cells from becoming detached into an abdominal cavity during a surgical operation of gastrointestinal cancers in mice.

23. The method of claim 17, wherein the at least one cancer tumor is a solid tumor selected from the group comprising: AIDS related cancers, osteosarcoma, and cancers of the anus, appendix, bile duct, bladder, brain, breast, cervix, colon, esophagus, eye, gall bladder, head, neck, heart, liver, kidney, larynx, lip, oral cavity, lung, mouth, paranasal sinus and nasal cavity, ovaries, pancreas, parathyroid, penis, prostate, rectum, salivary glands, skin, spleen, throat, testicles, urethra, and vagina, and renal cell carcinoma.

24. The method of claim 17, wherein the glue membrane is applied topically.

25. The method of claim 16, wherein application of the kit substantially inhibits the release of cytokines, including TNF (Tumor Necrosis Factor), and substantially limits the activation of histones and toxicity produced by one time radio-chemotherapy.

26. The method of claim 17 further comprising combining the glue membrane with a slow-release adjuvant agent such as Fluorouracil (C4H3FN202) for gastric cancer, Sorafenib for breast cancer, or other adjuvant instruments used to inhibit tumor cancer cells, including: Adriamycin, Daunorubicin, Etoposide, Irinotecan (Campto), Cyclophosphamide, Epirubicin, Docetaxel, (Taxotere) Paclitaxel, (Taxol), Ifosphamide, Vindesine, Vinorelbine, Topotecan, Amsacrine, Cytarabine, Bleomycin, Busulphan, 5F1uorourecil, Melphalan, Vincristine, Vinblastine, Lomustine(CCNU), Thiotepa, Gemcitabine, Methotrexate, Carmustine (BCNU+), Mitroxantrone, Mitomycin C, Carboplatin, Cisplatin, Procarbazine, 6-Mercaptopurine, Sreptozotocin, Fludarabine, Raltitrexate (Tomudex), Capecitabine.

27. The method of claim 4, wherein A1AT stabilizes fibrinogen, high concentrate fibrinogen, or a similar protein.

28. The method of claim 17, further comprising combining the glue membrane with an agent capable of inhibiting tumor cancer cells.

29. The method of claim 28, wherein the agents are Sorafenib for breast cancer or Fluorouracil for gastric cancer or any other cancer drug.

30. The kit (Fribinogen) is used alone, throat cancer patients will not lose their taste because no chemo radio therapy is used.

31. The kit (Fribinogen) is used alone will prevent solid tumor cancer patients from losing their hair during the treatment as no chemo radio therapy is used.

32. A glue membrane matrix comprised of A1AT enriched high concentrated fibrinogen, the glue membrane matrix further comprising a mesh, the mesh having a diameter less than 10 micrometers at its largest dimension.

33. A kit containing a glue membrane with isolated KH proteins, the isolated KH proteins having amino acid, sequences SEQ ID NO: 1, SEQ ID NO: 2, SEQ ID NO. 3, and SEQ ID NO: 4.

34. A kit containing thrombin and isolated KH proteins derived from KH healthy cells in thrombin, the isolated KH proteins having amino acid sequences SEQ ID NO: 5, SEQ ID NO: 6, SEQ ID NO: 7, and SEQ ID NO: 8, the isolated proteins inhibiting solid cancer cells and Blood (Liquid) cancers cells.

35. A kit containing high concentrate fibrinogen and isolated proteins derived from KH healthy cells in high concentrate fibrinogen, the isolated proteins having amino add sequences SEQ ID NO: 1, SEQ ID NO: 2, SEQ ID NO: 3, and SEQ ID NO: 4, the isolated proteins inhibiting solid cancer cells and Blood (liquid) cancers cells.

36. A kit containing a glue membrane wherein the glue membrane is enriched with 5% of liquid form A1AT diluent for high concentrate fibrinogen, enhancing the stability and durability of the glue membrane inside a body lumen up to 52 days.

37. A kit containing AFOD, RAAS 8 A1AT, and isolated proteins derived from KH healthy cells defined by SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18, the isolated proteins inhibiting solid cancer cells and blood (Liquid) cancers cells.

38. A compound comprising a fibrin sealant combined with tumor inhibiting agent Sorafenib as an adjuvant instrument to inhibit breast cancer cells.

39. A cancer tumor inhibiting compound comprised of fibrinogen, thrombin and high concentrated fibrinogen in which 5% of A1AT diluent is added, the compound containing KH proteins defined by: SEQ ID NO: 1, SEQ ID NO: 2, SEQ ID NO: 3, SEQ ID NO:5, SEQ ID NO: 7, SEQ ID NO: 8, and SEQ ID NO: 4.


SEQUENCE LISTING (18 sequences)

SEQ ID NO: 1 - protein, 420 aa, homo sapiens
NWIQYKVGPEADKYRVGPEADKYRVELEDWNGRVELEDWNGRTSTADYAMFKTSTADYAMFKDNCCILDERDNCCILDERQSGLYFIKPLKYEASILTHDSSIRYEASILTHDSSIRYLQEIYNSNNQKYLQEIYNSNNQKIHLISTQSAIPYALRIHLISTQSAIPYALRASTPNGYDNGIIWATWKASTPNGYDNGIIWATWKCHAGHLNGVYYQGGTYSKEGFGHLSPTGTTEFWLGNEKEGFGHLSPTGTTEFWLGNEKVELEDWNGRTSTADYAMFKFGSYCPTTCGIADFLSTYQTKAIQLTYNPDESSKPNMIDAATLKAIQLTYNPDESSKPNMIDAATLKAIQLTYNPDESSKPNMIDAATLKVAQLEAQCQEPCKDTVQIHDITGKVAQLEAQCQEPCKDTVQIHDITGK

SEQ ID NO: 2 - protein, 439 aa, homo sapiens
NWIQYKVGPEADKYRVGPEADKYRVELEDWNGRVELEDWNGRTSTADYAMFKTSTADYAMFKDNCCILDERDNCCILDERQSGLYFIKPLKYEASILTHDSSIRYEASILTHDSSIRYLQEIYNSNNQKYLQEIYNSNNQKIHLISTQSAIPYALRIHLISTQSAIPYALRASTPNGYDNGIIWATWKCHAGHLNGVYYQGGTYSKEGFGHLSPTGTTEFWLGNEKEGFGHLSPTGTTEFWLGNEKVELEDWNGRTSTADYAMFKMLEEIMKYEASILTHDSSIRFGSYCPTTCGIADFLSTYQTKAIQLTYNPDESSKPNMIDAATLKAIQLTYNPDESSKPNMIDAATLKAIQLTYNPDESSKPNMIDAATLKANQQFLVYCEIDGSGNGWTVFQKVAQLEAQCQEPCKDTVQIHDITGKVAQLEAQCQEPCKDTVQ

SEQ ID NO: 3 - protein, 274 aa, homo sapiens
TLLEGEESRAQYEDIAQKAQYEDIAQKTNAENEFVTIKNSKIEISELNRSLDLDSIIAEVKNMQDMVEDYRSKAEAESLYQSKLNDLEDALQQAKLNDLEDALQQAKTNAENEFVTIKKTNAENEFVTIKKLLRDYQELMNTKSGGGFSSGSAGIINYQRLDSELKNMQDMVEDYRAEAESLYQSKYEELQITAGRGSYGSGGSSYGSGGGSYGSGGGGGGHGSYGSGSSSGGYRGSYGSGGSSYGSGGGSYGSGGGGGGHGSYGSGSSSGGYR

SEQ ID NO: 4 - protein, 323 aa, homo sapiens
YLYEIARYLYEIARLCTVATLRDDNPNLPRKYLYEIARYLYEIARRLVNEVTEFAKLVNEVTEFAKFKDLGEENFKFKDLGEENFKETYGEMADCCAKETYGEMADCCAKETYGEMADCCAKTCVADESAENCDKQEPERNECFLQHKQEPERNECFLQHKSLHTLFGDKLCTVATLRNECFLQHKDDNPNLPRNECFLQHKDDNPNLPRTCVADESAENCDKSLHTLFGDKLVRPEVDVMCTAFHDNEETFLKLVRPEVDVMCTAFHDNEETFLKKLVRPEVDVMCTAFHDNEETFLKKLVRPEVDVMCTAFHDNEETFLKK

SEQ ID NO: 5 - protein, 592 aa, homo sapiens
SCHTAVGRASYLDCIRASYLDCIRKSCHTAVGRKASYLDCIRKASYLDCIRDSGFQMNQLRDSGFQMNQLRCQSFRDHMKDSGFQMNQLRDSGFQMNQLRCQSFRDHMKSASDLTWDNLKSASDLTWDNLKHSTIFENLANKEFQLFSSPHGKWCAVSEHEATKKDSGFQMNQLRDSAHGFLKVPPRKDSGFQMNQLRKSASDLTWDNLKCLKDGAGDVAFVKSVIPSDGPSVACVKMYLGYEYVTAIRSKEFQLFSSPHGKMYLGYEYVTAIRMYLGYEYVTAIRLKCDEWSVNSVGKDQYELLCLDNTRSVIPSDGPSVACVKKEDPQTFYYAVAVVKDCHLAQVPSHTVVARIECVSAETTEDCIAKEDPQTFYYAVAVVKKEGTCPEAPTDECKPVKADRDQYELLCLDNTRADRDQYELLCLDNTRMDAKMYLGYEYVTAIRSAGWNIPIGLLYCDLPEPRIMNGEADAMSLDGGFVYIAGKIMNGEADAMSLDGGFVYIAGKNLREGTCPEAPTDECKPVKDGAGDVAFVKHSTIFENLANKKPVDEYKDCHLAQVPSHTVVARKPVDEYKDCHLAQVPSHTVVAR

SEQ ID NO: 6 - protein, 271 aa, homo sapiens
IIAPPERKAGFAGDDAPRIKIIAPPERGYSFVTTAERGYSFVTTAERHQGVMVGMGQKHQGVMVGMGQKAVFPSIVGRPRAVFPSIVGRPRHQGVMVGMGQKDSYVGDEAQSKRQEYDEAGPSIVHRQEYDEAGPSIVHRLDLAGRDLTDYLMKGYSFVTTAEREIVRLDLAGRDLTDYLMKSYELPDGQVITIGNERSYELPDGQVITIGNERVAPEEHPTLLTEAPLNPKVAPEEHPTLLTEAPLNPKDLYANNVLSGGTTMYPGIADR

SEQ ID NO: 7 - protein, 333 aa, homo sapiens
QKVEPLRAELQEGARAKPALEDLRAKPALEDLRLSPLGEEMRQKLHELQEKATEHLSTLSEKVQPYLDDFQKWQEEMELYRWQEEMELYRTHLAPYSDELRTHLAPYSDELRETEGLRQEMSKLSPLGEEMRDRLSPLGEEMRDRETEGLRQEMSKVQPYLDDFQKKVQPYLDDFQKKDYVSQFEGSALGKKWQEEMELYRVKDLATVYVDVLKVEPLRAELQEGARVEPLRAELQEGARTHLAPYSDELRQRLLDNWDSVTSTFSKDLATVYVDVLKDSGRDSGRDYVSQFEGSALGKDSGRDYVSQFEGSALGKLREQLGPVTQEFWDNLE

SEQ ID NO: 8 - protein, 105 aa, homo sapiens
IIAPPERKAGFAGDDAPRIKIIAPPERGYSFVTTAERGYSFVTTAERAVFPSIVGRPRAVFPSIVGRPRHQGVMVGMGQKDSYVGDEAQSKRQEYDEAGPSIVHR

SEQ ID NO: 9 - protein, 1148 aa, homo sapiens
CQNMLRNPNKDVRVSWIKDKELLDLHKQPTEAMMGLAQLYKEAMMGLAQLYKIYAPEAPYTSHDKLKIYAPEAPYTSHDKLKKGENGNGRNYPLEKRNYPLEKRMASRNYPLEKMASRNYPLEKMQLLSLRQLGAQRTSSSTQLKSEGSGGTQLKSSLSTSSPESARSSLSTSSPESARSKIPTYCSICCEQRYTKHKANAYKETELDIRTCRETELDIRAMQTGVTGIMIAREQRHWDISSSERMAEGTAEAPLENGGGGDSGAGALERSQPVHILAVDKVEQTPRMQAAGPLFRMQAAGPLFRSKGQCLSSRRDHLQRMDPGSRWRWRNLPSGPSLKWRNLPSGPSLKALEGRVFKALEGRVFKAEGFLVAEKQNLMTFGTPVLRMLDLVQAQILELKAQSQTDRMAVMAPRESRSLSGDLNVKAQSQTDRNVKAQSQTDRDYIALNEDLREGDGGVMSLRESRRPCGMGKRPCGMGKFLRSCVWDGNTPLKKSVGPETAENESKLLMKSVGPETAENESKLLMKLEMHGLWEGSQEGDGWQRADMSPCPHLPLITPGICMFKIYADPTKIYADPTKRANISVHPDKLKFSTSSLLLRIRDPYCHPVCANRNAVKANISVHPDKEPMNPIKQYVKDGKLYKVPDGKLYKVPDGKFHDIDDVKILSCGEVIHVKILSCGEVIHVKRAMALYHTEERTSSSTQLKSEGSGGTQLKSSLSTSSPESARSSLSTSSPESARSKIPTYCSICCMTEEFRLLCMYRLKICYFMRLKICYFMRWYLSSTELAEKMTEEFRLLCMYRLKICYFMRLKICYFMRWYLSSTELAEKLCQMEAFMRKLCQMEAFMRKLCQMEAFMRGPPHGWPKGWGRNYASIIRCVCAGCRAAPSRRDCMWSGFSARMCVCAGCRAAPSRQNESVSRVTLGENSRAEIENEDYSAMCALLNSGGGVIKACASFIRRQEYQKTQQEIEDKRSGPPASYCAKALQEYSSISEKALQEYSSISEKALTNIQNCMAEKLPDGDCRRLPGVNSKGVCQPPVQGRPPLDVSWWKTFVQSKKDLLQAKREIEFLPSRTSLYSEDDCKFKTQEYPEGRFKTQEYPEGRDEFAEDRIYRDEFAEDRIYRWMLAGRPHPTLKGFSGGMKGCLGTWR

SEQ ID NO: 10 - protein, 1255 aa, homo sapiens
RGEDYLKYDPRHNRHFTASLAPRMYITCGRRNFVAFSRTDKAWHGMATLLNKDTVASALMYHRILLAASCDYFRCQGAKHNGKHNGKGGADAAEGSPDAHHPGLFMFEPILEEKMESLSSRMSGRVGDLSPKVGDLSPKQAETLAKVGDLSPKQAETLAKMEEEGGAMEKMMEEEGGAMEKMEEEGGAMEKVSKMEEEGGAMEKVSKSGGYGFGRSLNVASGSGKGDNALKDARNEISELTRNEISELTRSRGSANDYKQLETLSGDRQLETLSGDRKQLETLSGDRNNLEPILEGYISNLRKVTPCSECKSPAREPGDVSARDLGSDAGSSSSSSRDLGSDAGSSSSSSRMDLGSDAGSSSSSSRRGEDYLKYDPRHNRHFTASLAPRMYITCGRRNFVAFSRTDKAWHGMATLLNKDTVASALMYHRILLAASCDYFRGLEAADKKESEEGCPKLFDDSDERGFEGSCSQKMQELYGDGKVFDDESDEKGDGLCCYLKRARHFSEHPSTSKTEDGGEFEEGASENNAKAGELADKKMLANFESGKMLANFESGKFYGDLMEKDASPSKEERDIIGVAETGSGKEDYSITTKGGKVFLMSESEKRAGAKDILVATDVAGRAGAKDILVATDVAGRIEESDQGPYAIILAPTRCNTPTYCDLGKMCNTPTYCDLGKMCNTPTYCDLGKAAKVTPCSECKSPAREPGDVSARDLGSDAGSSSSSSRDLGSDAGSSSSSSRMDLGSDAGSSSSSSRNRLICRWVSSRERNEEAQVHDKEMESPECMKEMESPECMKYGTDLLLYRKNAEDIEQLYGKEMESPECMKREMESPECMKREMESPECMKREAGGCVRGRAESRSGAQRRAPAPAGQQQMRGGGGVSFTKACPWREPIREVDTNVATGAPPRHCSAPEDPIFRAGELADKKMLANFESGKMLANFESGKFYGDLMEKDASPSKEERDIIGVAETGSGKEDYSITTKGGKVFLMSESEKRAGAKDILVATDVAGRAGAKDILVATDVAGRIEESDQGPYAIILAPTRCCKATEKDVASCIDREYFDEMKEYFDEMKVECANNALKVECANNALKDQIQAELDSKFNDDFIETRRFNDDFIETRRDVASCIDRCCKMGYMMAKAEVDKDDKMGYMMAKKLDARNSRYRDLQFRDRDCSGAFRCKCVPQMQQSREEAGDIASKGNAADAESFREEAGDIASKSESKEESEVRSWDWVHGNRLGRMPGLGFDKMPGLGFDKHGNR

SEQ ID NO: 11 - protein, 2895 aa, homo sapiens
HLYEEKHLYEEKDPQRWKTSENEAIKDFSKNLEKELDSLSKVKELDSLSKVKGSELRMINKFLLSLQKDKKLDELPIFKSSEFLKHAGKWMTKHKSLKFTNAKFVKWMTKALSYLYEKSQFEKFVRNNEPYDYKMSLKFTNAKFVRFCFIKLSAIPVSAFCNSETKTITIGSAGKTFSVTGWKTITIGSAGKTFSVTGWKRPLVSSRELDNVTKELDNVTKNILQTLKVLWEQRLLSTLGPRLQTEACRQHLLPGGRCQAMLLRHALQLAQKAHQGQVQRELLGAAGHRMAGQPHSPRKHWEDLRNRDPEEVKGQELEDRRYPHIQAQRRQEEVTVRATSLPNNVARDARPGCLLRDARPGCLLRQVQEQVAQRAQMDTSPAPRAQMDTSPAPRSISDLSLQLKMERWLCSKHVEALQQVGRLQTALQAQNREALIQALDCGKALGSTAAQSLSPKLEEILAAQEVSLKAGQPHSPRELLGAAGHRAGQPHSPRELLGAAGHRWMTKHKILGFSTSAKILGFSTSAKFVKWMTKALSYLYEKSQFEKFVRNNEPYDYKMSLKFTNAKFVRFCFIKLSAIPVSAFCNSETKTITIGSAGKTFSVTGWKTITIGSAGKTFSVTGWKVCGNKIKVCGNKIKVLKSIMNKNEMEKQRNPAFEGVNRMLEAENALKMLEAENALKVVDHAEENKATSFEESERFVQPLLGRKFVQPLLGRKICASEIENRNIVSWVEKKLLLASPPNEREAIFSDCFKVLKSIMNKNEMEKQRMLEAENALKMLEAENALKFVQPLLGRKFVQPLLGRKNIVSWVEKKEAIFSDCFKHLYEEKHLYEEKDPQRWKELKYCKGRDAVGFRGRDAVGFRTSENEAIKITTTEDFKDFSKNLEKELDSLSKVKELDSLSKVKGSELRMINKFLLSLQKDKKLDELPIFKSSEFLKHAGKDPEGKIYYYHVITRWTVNGQLRVGFFTTKHLYEEKHLYEEKDPQRWKTNLSYSRTSENEAIKDFSKNLEKDTRYVSSRELDSLSKVKELDSLSKVKQLQPQPPPKSHYYDSDRGSELRMINKDLRTSSYSKFLLSLQKDKKLDELPIFKSSEFLKHAGKGPMFKGVASSRWTVNGQLRVGFFTTKWTVNGQLRVGFFTTKHLYEEKHLYEEKMTPLPYRMTPLPYRMTPLPYRSRMTPLPYRSRSEVSKLRSEVSKLRQNLNVDKSRAEELKMRGMMAKSLEESQGKSLEESQGKFSENGNQKFSENGNQKTNELLKSKILSLELMKMSFPKAPLKSALKETEIKCQLLEKEKEITVSFLQKQLEEEAKSRQLEEEAKSRQLEDEEGRKIQDLETELEKDENSQLKSEVSKHLYEEKHLYEEKTNLSYSRTSENEAIKDTRYVSSRMFKGVASSRELDSLSKVKELDSLSKVKSHYYDSDRGSELRMINKDLRTSSYSKFLLSLQKDKKLDELPIFKSSEFLKHAGKILNSSEGVKEYLSLSDKKHSQENIKSHHFEDSKLSKSEMHRFHVHFCRMSPEKQHRERQPFLVKSKITVPFLKQMKEELQKQMKEELQKDTLEFEMRDTLEFEMRMSIEMEAMKMSIEMEAMKMSIEMEAMKEKSSLGNELKNITDLKFHSRHLYEEKHLYEEKDPQRWKTNLSYSRGRDAVGFRGRDAVGFRTSENEAIKITTTEDFKDFSKNLEKDTRYVSSRELDSLSKVKELDSLSKVKQLQPQPPPKSHYYDSDRGSELRMINKDLRTSSYSKFLLSLQKDKKLDELPIFKSSEFLKHAGKGPMFKGVASSRDPEGKIYYYHVITRWTVNGQLRVGFFTTKMAQTQGTRVMTVSFHKYHSDDYIKMAQTQGTRRMEIYRPHKMEIYRPHKKMEIYRPHKSIRPDNMSEYSKMTHNLLLNYGLYRSEVSKLRSEVSKLRQNLNVDKSRAEELKMRGMMAKSLEESQGKSLEESQGKFSENGNQKFSENGNQKTNELLKSKILSLELMKMSFPKAPLKSALKETEIKCQLLEKEKEITVSFLQKQLEEEAKSRQLEEEAKSRQLEDEEGRKIQDLETELEKDENSQLKSEVSKHLYEEKHLYEEKDPQRWKTNLSYSRTSENEAIKDFSKNLEKDTRYVSSRELDSLSKVKELDSLSKVKQLQPQPPPKSHYYDSDRGSELRMINKDLRTSSYSKFLLSLQKDKKLDELPIFKSSEFLKHAGKGPMFKGVASSRRASVTERRASVTERGGSTTANNRGGSTTANNRFINTESGRFINTESGRLGSKVEVSKQQQQQPQKAGPQPCYLKAGPQPCYLKTKPSAPAGALKIPKSSALVSRPAILVASKMKRAPWEDPAKRAPWEDPAKGNGTAQSADLRGNGTAQSADLREGLQLYGRRSQSFNNYDKDSELNELRKYPDVASPTLRNSLWSGDDVKTRGGSTTANNRQVPIATAENMKLSSGLPQQKPQLDGRRASVTERRASVTERGGSTTANNRGGSTTANNRFINTESGRFINTESGRLGSKVEVSKQQQQQPQKAGPQPCYLKAGPQPCYLKTKPSAPAGALKIPKSSALVSRPAILVASKMKRAPWEDPAKRAPWEDPAKGNGTAQSADLRGNGTAQSADLREGLQLYGRRSQSFNNYDKDSELNELRKYPDVASPTLRNSLWSGDDVKTRGGSTTANNRQVPIATAENMKLSSGLPQQKPQLDGRNLILQMLRLMPSLSAAGRLMPSLSAAGRQHQSLQITTVMVAVKIISKSEVSKLRSEVSKLRQNLNVDKSRAEELKMRGMMAKSLEESQGKSLEESQGKFSENGNQKFSENGNQKTNELLKSKILSLELMKMSFPKAPLKSALKETEIKCQLLEKEKEITVSFLQKQLEEEAKSRQLEEEAKSRQLEDEEGRKIQDLETELEKDENSQLKSEVSK

SEQ ID NO: 12 - protein, 2375 aa, homo sapiens
KSSMVALKYKLLESKNLEVEHRLRQQAEIKLRQQAEIKESSLSRQSKNYEALKQRQKGQLEDLEKQKGQLEDLEKETEVLQTDHKQASEYESLISKDLEDRYNQLLKDLEDRYNQLLKSVSGKTPGDFYDRSSSQENLLDEVMKTLVTLREDLVSEKLIEVERNNATLQAEKKTEDTYFISSAGKPTPGTQGKTLLEQNMESKDLFHVEQRTLLEQNMESKDLFHVEQRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRAAQIAGAVRATPAHRARGNMRSCRGNMRSCRLTDFGFGRSCRVLLHMRSCRVLLHMRGHQGGGPAASAPGLRGAPGHPLRPQEVRCENVLLSPDERRLEAGWFQPFLQPRALGQGGARLMALGPIRLMALGPIRFVTISGQKDFTELQKKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRQLNLLQKRFSNIDKFALTAETKRNFSNMKSLTGYLEKSLTGYLEKAELEHWKIPAWWKKRLELWLRRLELWLRVNDLIEFRSVAMMVPDRSVAMMVPDRTWREMDIRNMKLIESKPKNMKLIESKPKAVLLIGEQGTAKLLSDIFIPALRLLSDIFIPALRVDTLHYAWEKTSIIDFTVTMKLKEASESVAALSKRLLSDIFIPALRLPNPAYTPEISARLVSVLSTIINSTKKNFSNMKMMLAEYQRERWTEQSQEFAAQTKELLSHFNHQNMDALLKVTREAALTLPREAALTLPREILNNHGKDAAPGASKLRDAAPGASKLRGVVDHLLLRGVVDHLLLRQPWHGKAMQRNNEFPVFDEFNNEFPVFDEFGGSPAVTLLISEKASEAGATAPKASARISAPNEFDVMFKLSRDDISTAAGMVKDDVDMLRDYDALRKLEQEYSRRENGQLLRRENGQLLRAHGPEVQAHNKAHGPEVQAHNKKAANEEMEALRLADVEQELSFKKLADVEQELSFKKDDNSATKTLSAAARLQTEVELAESKLKAANEEMEALRQIKVQKGSEPLGISIVSGEKDLNPGVKKDLNPGVKKMSLGQLQSARGTCSGFEPHSWRGVACLGCKGTCSGFEPH

SEQ ID NO: 13 - protein, 2379 aa, homo sapiens
RQAVQDLRADVVNKALREKMELEMRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRSWCQELEKRGEHGRLLSLWRVNVELQLQGDSAQGQKLMALGPIRDFTELQKYSEIQDRYSEIQDRKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKLMALGPIRDFTELQKYSEIQDRYSEIQDRKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKLMALGPIRDFTELQKKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKTNFPYVRSDILGHLRSDILGHLRNIFKRPLGSRNIFKRPLGSRQSGVVPFIFQAKSGFPLLKEFVQRTLSGTPEVHSNKRTLSGTPEVHSNKRAAEGIPKLLVLITGGKYPPPGEMGASEVLLGAFSIKMKPLDGSALYTGSALDFVRSAGSRIEDGVLQFLVLLVAGRVDGPASNLKQSGVVPFIFQAKGNRVSPTMKDSLLELSPVERDSLLELSPVERYPGMFIALSKNGKYPGMFIALSKNGKRLYCNVGIGFHLQALPDGRRLYCNVGIGFHLQALPDGRLFEDMVTKMAGASPAVPHERARMAGASPAVPHERARLFEDMVTKLQALRALGVGGAGCGVQGLASLARTTGLSDQQVVCDLDHRAVEALLQAVRMNGEYRGRGGGNFSGKWRTSEQTPQFLLSTKELTEGGEVTNLIPDIATELRTLACMGLAIHQVLTKDLERDAPEKTLACMGLAIHQVL

SEQ ID NO: 14 - protein, 2104 aa, homo sapiens
RQAVQDLRRQAVQDLREKMELEMRLKNEEVESERIQVLEDQRTRLSALNEALALDKLHMEKADVVNKHFLEMKSATDRQDYITRSAQTSRQDYITRSAQTSRQDLAEQLQGLSSAKELTTQRQLMQERERLQEMLMGLEAKERVSLLETLLQTQKHNVQLRSTLEQVERVNVELQLQGDSAQGQKLSGSLLTCCLRLTVGAQSRRQAVQDLRRQAVQDLRLKNEEVESERIQVLEDQRTRLSALNEALALDKLHMEKADVVNKHFLEMKSATDRQDYITRSAQTSRQDYITRSAQTSRQDLAEQLQGLSSAKELTTQRQLMQERERLQEMLMGLEAKERVSLLETLLQTQKHNVQLRSTLEQVERVNVELQLQGDSAQGQKLSGSLLTCCLRLTVGAQSRRQAVQDLRRQAVQDLRLHMEKADVVNKHFLEMKSATDRLQSSQLQSCRVLKVNVELQLQGDSAQGQKLSGSLLTCCLRLTVGAQSRWHVVSSKLMALGPIRYSEIQDRYSEIQDRQQVQFMLKQQVQFMLKVVKAQIQEQKNCPISAKLERQQLEETSEIRQISEQLNALNKQISEQLNALNKLLEVWIEFGRGDLRFVTISGQKQPVYDTTIRTGRQPVYDTTIRTGRARQEQLELTLGRTGSLEEMTQRLRNTISVKAVCDYRNTISVKAVCDYRLNDALDRLEELKFEQLCLQQQEKEETYNQLLDKGRLMLLSRDDSGSGSKQTTGEEVLLIQEKLLEVWIEFGRIKLLEVWIEFGRIKELNPEEGEMVEEKEIKFLDVLELAEKNDECVLEDNSQRTKEGLDKLVSDANEQYKHMLEEEGTLDLLGLKKLLPQAEMFEHLSGKLDQMVFFWEDIKARALIAEHQTFMEEMTRKALIAEHQTFMEEMTRKDENYYQLEELAFRVMRLLSDTVASDPGVLQEQLATTKMSELRVTLDPVQLESSLLRSTVMVRVGGGWMALDEFLVKFSQQYSTIVKDYELQLMTYKAEFFADVVPAVRDILFPYIEENVKMKAEFFADVVPAVRQLQGHMWRAAFTAGRLLFGHSTEGDILELVDGHFDTKIGHKVYIYSSGSVEAQKLLFGHSTEGDILELVDGHFDTKSVAYMPYAEVKSVAYMPYAEVKSVAYMPYAEVKRSSTSPCGTSKSPNRSSTSPCGTSKSPNRRALEQEAQMHNTAARYSVPPVLQPAPHQVITNLPEWHVVSSKLMALGPIRYSEIQDRYSEIQDRQQVQFMLKQQVQFMLKVVKAQIQEQKNCPISAKLERQQLEETSEIRQISEQLNALNKQISEQLNALNKLLEVWIEFGRGDLRFVTISGQKQPVYDTTIRTGRQPVYDTTIRTGRARQEQLELTLGRTGSLEEMTQRLRNTISVKAVCDYRNTISVKAVCDYRLNDALDRLEELKFEQLCLQQQEKEETYNQLLDKGRLMLLSRDDSGSGSKQTTGEEVLLIQEKLLEVWIEFGRIKLLEVWIEFGRIKELNPEEGEMVEEKEIKFLDVLELAEKNDECVLEDNSQRTKEGLDKLVSDANEQYKHMLEEEGTLDLLGLKKLLPQAEMFEHLSGKLDQMVFFWEDIKARALIAEHQTFMEEMTRKALIAEHQTFMEEMTRKDENYYQLEELAFRVMRLLSDTVASDPGVLQEQLATTKMSELRVTLDPVQLESSLLRSTVMVRVGGGWMALDEFLVKFSQQYSTIVKDYELQLMTYKIAEIFMRHLFCPDLLRHLFCPDLLRDKVAFITGGGSGIGFRIAEIFMRAEFFADVVPAVRDILFPYIEENVKMKAEFFADVVPAVRQLQGHMWRAAFTAGRLLFGHSTEGDILELVDGHFDTKIGHKVYIYSSGSVEAQKLLFGHSTEGDILELVDGHFDTKKLPASEGVGKKLPASEGVGKNVILMAHDGKFYMVSYYERFYMVSYYERWQDRCLEVRGPITAVAFAPDGRSLAALKNMAHHKGLQECFPAICRYSHNSLMVQAIKYSHNSLMVQAIKLPASCLPASDSFRVIIWTENGQSYIYKHTCKALTFLLLQPPSPKEHLLDDEEEDEEIMRQRYDQRYLISGGVDFSVIIWDIFSGEMKQGWSQLAAMHCVMLPDLLGLDKFRPPLLEMLAR

SEQ ID NO: 15 - protein, 2591 aa, homo sapiens
KLPASEGVGKFYMVSYYERWQDRCLEVRWQDRCLEVRGPITAVAFAPDGRSLAALKNMAHHKSLAALKNMAHHKGLQECFPAICRYSHNSLMVQAIKYSHNSLMVQAIKLPASCLPASDSFRVIIWTENGQSYIYKEHLLDDEEEDEEIMRHTCKALTFLLLQPPSPKHALSLIATARPPAFITTIAKEHLLDDEEEDEEIMRQREHLLDDEEEDEEIMRQRCQTIHGHKGPITAVAFAPDGRYDQRYLISGGVDFSVIIWDIFSGEMKKLPASEGVGKFYMVSYYERWQDRCLEVRWQDRCLEVRGPITAVAFAPDGRSLAALKNMAHHKSLAALKNMAHHKGLQECFPAICRYSHNSLMVQAIKYSHNSLMVQAIKLPASCLPASDSFRVIIWTENGQSYIYKEHLLDDEEEDEEIMRHTCKALTFLLLQPPSPKHALSLIATARPPAFITTIAKEHLLDDEEEDEEIMRQREHLLDDEEEDEEIMRQRCQTIHGHKGPITAVAFAPDGRYDQRYLISGGVDFSVIIWDIFSGEMKEVQLVESGAEVKSDDTAVYYCARSDDTAVYYCARASGYTFTGYYMHWVRVTMTRDTTISTAYMELSRIAAAGDAFDIWGQGTMVTVSSLMALGPIRLMALGPIRYSEIQDRQQVQFMLKQQVQFMLKVVKAQIQEQKNCPISAKLERNCPISAKLERMPPLIPAEVDKQQLEETSEIRQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKWADHEVRAMESTATAAVAAELVSADKMAMESTATAAVAAELVSADKAMESTATAAVAAELVSADKMSGRLMALGPIRLMALGPIRYSEIQDRQQVQFMLKQQVQFMLKVVKAQIQEQKNCPISAKLERNCPISAKLERMPPLIPAEVDKQQLEETSEIRQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKLMALGPIRLMALGPIRQQVQFMLKQQVQFMLKVVKAQIQEQKNCPISAKLERNCPISAKLERMPPLIPAEVDKQQLEETSEIRQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKSYAEELAKISYSLFTALRIVMNMNKSTRDKLDQVSSEIKDKLDQVSSEIKMININILSVCKMININILSVCKGVFVQSVLPYFVATKLAKMININILSVCKMTQLVLPGMVERMININILSVCKMTQLVLPGMVERYEDEINKEDLARLLRGFSSGSAVVSGGSRRSTSSFSCLSRHGGGGGGFGGGGFGSRHGGGGGGFGGGGFGSRGSSSGGGYSSGSSSYGSGGRQCKNVQDAIADAEQRSISISVAGGGGGFGAAGGFGGRDYQELMNVKLALDVEIATYRNPGSLRGRLETHPCRGSIGQSAIPRSPCPIRSPLPARASAPWASLSTRADSGLRMSPLETNKGSIGQSAIPRATSASLPQETPFALSVVWAPRR

SEQ ID NO: 16 - protein, 219 aa, homo sapiens
ALPAPIEKNQVSLTCLVKEPQVYTLPPSREPQVYTLPPSRFNWYVDGVEVHNAKFNWYVDGVEVHNAKGQPREPQVYTLPPSREPQVYTLPPSRDELTKEPQVYTLPPSRDELTKTPEVTCVVVDVSHEDPEVKTPEVTCVVVDVSHEDPEVKGRFTISGDISTNTLYLQMHSLRGFYPSDIAVEWESNGQPENNYKGFYPSDIAVEWESNGQPENNYK

SEQ ID NO: 17 - protein, 378 aa, homo sapiens
IIAPPERKAGFAGDDAPRAGFAGDDAPRDLTDYLMKIKIIAPPERGYSFTTTAERGYSFTTTAERHQGVMVGMGQKHQGVMVGMGQKAVFPSIVGRPRAVFPSIVGRPRHQGVMVGMGQKDSYVGDEAQSKRIWHHTFYNELRQEYDESGPSIVHRQEYDESGPSIVHRLDLAGRDLTDYLMKGYSFTTTAEREIVRLDLAGRDLTDYLMKQEYDESGPSIVHRKSYELPDGQVITIGNERSYELPDGQVITIGNERVAPEEHPVLLTEAPLNPKVAPEEHPVLLTEAPLNPKDLYANTVLSGGTTMYPGIADRDLYANTVLSGGTTMYPGIADRDLYANTVLSGGTTMYPGIADRKDLYANTVLSGGTTMYPGIADR

SEQ ID NO: 18 - protein, 264 aa, homo sapiens
ALDLINKRHSHESQDLRDGYLFQLLRDGYLFQLLRHPNVFGFCRHPNVFGFCRRDGYLFQLLRRPSEIVIGQCKRPSEIVIGQCKYWNDCEPPDSRGGEGTGYFVDFSVRGGEGTGYFVDFSVRYKEENDDFASFRYKEENDDFASFRKYWNDCEPPDSRKYWNDCEPPDSRDSPVLIDFFEDTERDSPVLIDFFEDTERVRGGEGTGYFVDFSVRVRGGEGTGYFVDFSVRADLFYDVEALDLESPKDSPVLIDFFEDTERYR